(19) United States
(12) Patent Application Publication
(10) Pub. No.: US 2023/0135171 A1
(43) Pub. Date: May 4, 2023

(54) METHODS AND SYSTEMS FOR MOLECULAR DISEASE ASSESSMENT VIA ANALYSIS OF CIRCULATING TUMOR DNA

(71) Applicant: Lexent Bio, Inc., Cambridge, MA (US)

(72) Inventors: Alex ROBERTSON, Princeton, NJ (US); Neil PETERMAN, San Francisco, CA (US); Nicole LAMBERT, San Diego, CA (US); Haluk TEZCAN, Cambridge, MA (US); Rohith SRIVAS, San Mateo, CA (US); Peter GEORGE, Cambridge, MA (US); Jason CLOSE, Cambridge, MA (US)

(73) Assignee: Lexent Bio, Inc., Cambridge, MA (US)

(21) Appl. No.: 17/788,221

(22) PCT Filed: Dec. 23, 2020
(86) PCT No.: PCT/US2020/066976

§ 371 Date: Jun. 22, 2022

Related U.S. Application Data
(60) Provisional application No. 62/993,564, filed on Mar. 23, 2020.
(60) Provisional application No. 62/953,368, filed on Dec. 24, 2019.

(51) Int. Cl. C12Q 1/6886; G16B 20/10; G16B 30/10; C12Q 1/6844

(52) U.S. Cl. CPC C12Q 1/6886; G16B 20/10; G16B 30/10; C12Q 1/6844; C12Q 2600/118; C12Q 2600/156

(57) ABSTRACT

The present disclosure provides methods of assessing tumor status (e.g., progression, regression, recurrence, etc.) in a subject. In an aspect, a method for assessing tumor status (e.g., progression, regression, recurrence, etc.) of a subject may comprise: based on first and second WGS data of cfDNA molecules of a subject at different time points, determing (i) a first and second plurality of CNAs and (ii) a first and a second plurality of fragment lengths; processing the first and second plurality of CNAs to determine a CNA profile change; comparing the first and second plurality of fragment lengths to determine a fragment length profile change; determining a first or second tumor fraction of the subject at the first or second timepoint, based at least in part on the CNA profile change and the fragment length profile change; and detecting a tumor status of the subject based at least in part on the first or second tumor fraction.

### CROSS REFERENCE TO RELATED APPLICATIONS

This application claims the priority benefit of U.S. Provisional Patent Application Nos. 62/953,368, filed Dec. 24, 2019, and 62/993,564, filed Mar. 23, 2020, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 197102004840SEQLIST.TXT, date recorded: Dec. 18, 2020, size: 34 KB).

### BACKGROUND

Tumor progression may generally refer to cases in which subjects (e.g., patients) with cancer have a tumor that is progressing in severity (e.g., tumor burden, tumor size, cancer stage). For example, tumor progression in a patient may be an indication that the patient's tumor is not responsive to a therapeutic regimen for the cancer. On the other hand, tumor non-progression in a patient may be an indication that the patient's tumor is responding to a therapeutic regimen for the cancer. In addition, the tumor progression or tumor non-progression status of a patient may be indicative of a prognosis of a subject for cancer treatments.

### SUMMARY

Methods and systems are provided for assessing tumor status (e.g., progression, regression, recurrence, etc.) of a subject, such as a patient with cancer, by analyzing a bodily fluid sample (e.g., blood sample) of the subject. Tumor progression or tumor non-progression may be assessed and/or monitored by analyzing tumor DNA (e.g., from cell-free DNA) from a sample of a subject. The tumor progression or tumor non-progression status of a subject may be indicative of a diagnosis, prognosis, or treatment selection for a subject with cancer.

In an aspect, the present disclosure provides a method for assessing tumor progression of a subject with cancer, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; processing the first WGS data to determine (i) a first plurality of copy number aberrations (CNAs) in the first plurality of cfDNA molecules and (ii) a first plurality of fragment lengths of the first plurality of cfDNA molecules; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; processing the second WGS data to determine (iii) a second plurality of copy number aberrations (CNAs) in the second plurality of cfDNA molecules and (iv) a second plurality of fragment lengths of the second plurality of cfDNA molecules; processing the first plurality of CNAs with the second plurality of CNAs to determine a CNA profile change; processing the first plurality of fragment lengths with the second plurality of fragment lengths to determine a fragment length profile change; determining a first tumor fraction of the subject at the first timepoint or a second tumor fraction of the subject at the second timepoint, based at least in part on the CNA profile change and the fragment length profile change; and detecting a tumor progression of the subject based at least in part on the first tumor fraction or the second tumor fraction.

In an aspect, the present disclosure provides a method for assessing tumor status (e.g., tumor progression, non-progression, regression, or recurrence) of a subject with cancer, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; determining, based on the first WGS data, (i) a first plurality of copy number aberrations (CNAs) in the first plurality of cfDNA molecules and (ii) a first plurality of fragment lengths of the first plurality of cfDNA molecules; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; determining, based on the second WGS data, (iii) a second plurality of copy number aberrations (CNAs) in the second plurality of cfDNA molecules and (iv) a second plurality of fragment lengths of the second plurality of cfDNA molecules; comparing the first plurality of CNAs with the second plurality of CNAs to determine a CNA profile change; determining a fragment length profile change based on the first plurality of fragment lengths and the second plurality of fragment lengths; determining a first tumor fraction of the subject at the first timepoint or a second tumor fraction of the subject at the second timepoint, based at least in part on the CNA profile change and the fragment length profile change; and detecting a tumor status (e.g., tumor progression, non-progression, regression, or recurrence) of the subject based at least in part on the first tumor fraction or the second tumor fraction.

In an aspect, the present disclosure provides a method for treating cancer in a subject, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; determining, based on the first WGS data, (i) a first plurality of copy number aberrations (CNAs) in the first plurality of cfDNA molecules and (ii) a first plurality of fragment lengths of the first plurality of cfDNA molecules; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; determining, based on the second WGS data, (iii) a second plurality of copy number aberrations (CNAs) in the second plurality of cfDNA molecules and (iv) a second plurality of fragment lengths of the second plurality of cfDNA molecules; comparing the first plurality of CNAs with the second plurality of CNAs to determine a CNA profile change; determining a fragment length profile change based on the first plurality of fragment lengths and the second plurality of fragment lengths; determining a first tumor fraction of the subject at the first timepoint or a second tumor fraction of the subject at the second timepoint, based at least in part on the CNA profile change and the fragment length profile change; detecting a tumor status (e.g., tumor progression, non-progression, regression, or recurrence) of the subject based at least in part on the first tumor fraction or the second tumor fraction; and, based on the detected tumor status, administering a therapeutically effective dose of a treatment (e.g., surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor) to treat the cancer of the subject. In some embodiments, the detected tumor status comprises tumor progression, and the method comprises administering a second treatment to the patient, wherein prior to the administration, the patient has been treated with a first treatment for the cancer (and the first and second treatments are different).

In some embodiments, the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid. In some embodiments, obtaining the first WGS data comprises sequencing the first plurality of cfDNA molecules to generate a first plurality of sequencing reads, or wherein obtaining the second WGS data comprises sequencing the second plurality of cfDNA molecules to generate a second plurality of sequencing reads. In some embodiments, the sequencing is by Nanopore sequencing, chain termination (Sanger) sequencing, sequencing by synthesis (e.g., Illumina or Solexa sequencing), single molecule real-time sequencing, massively parallel signature sequencing, Polony sequencing, 454 pyrosequencing, combinatorial probe anchor synthesis, sequencing by ligation (SOLiD sequencing), or GenapSys sequencing. In some embodiments, the sequencing comprises whole genome bisulfite sequencing (WGBS), whole genome enzymatic methyl-seq, whole exome sequencing, whole epigenome sequencing, methylation array, reduced representation bisulfite sequencing (RRBS-Seq), TET-assisted pyridine borane sequencing (TAPS), Tet-assisted bisulfite sequencing (TAB-Seq), APOBEC-coupled epigenetic sequencing (ACE-seq), oxidative bisulfite sequencing (oxBS-Seq), pull-down or methylated DNA immunoprecipitation sequencing, or cytosine 5-hydroxymethylation sequencing (e.g., via Bluestar).

In some embodiments, the sequencing is performed at a depth of no more than about 40×. In some embodiments, the sequencing is performed at a depth of no more than about 30×. In some embodiments, the sequencing is performed at a depth of no more than about 25×. In some embodiments, the sequencing is performed at a depth of no more than about 20×. In some embodiments, the sequencing is performed at a depth of no more than about 12×. In some embodiments, the sequencing is performed at a depth of no more than about 10×. In some embodiments, the sequencing is performed at a depth of no more than about 8×. In some embodiments, the sequencing is performed at a depth of no more than about 6×. In some embodiments, the sequencing is performed at a depth of no more than about 5×, no more than about 4×, no more than about 3×, no more than about 2×, or no more than about 1×.

In some embodiments, the method further comprises aligning the first or second plurality of sequencing reads to a reference genome, thereby producing a plurality of aligned sequencing reads. In some embodiments, the method further comprises enriching the first or second plurality of cfDNA molecules for a plurality of genomic regions. In some embodiments, the enrichment comprises amplifying the first or second plurality of cfDNA molecules. In some embodiments, the amplification comprises selective amplification. In some embodiments, the amplification comprises universal amplification. In some embodiments, the enrichment comprises selectively isolating at least a portion of the first or second plurality of cfDNA molecules. In some embodiments, selectively isolating the at least the portion of the first or second plurality of cfDNA molecules comprises using a plurality of probes, each of the plurality of probes having sequence complementarity with at least a portion of a genomic region of the plurality of genomic regions. In some embodiments, the at least the portion comprises a tumor marker locus. In some embodiments, the at least the portion comprises a plurality of tumor marker loci. In some embodiments, the plurality of tumor marker loci comprises one or more loci having copy number alteration (e.g., CNA loci such as MET, EGFR, and BRCA2, and whole arm CNAs in chromosomes 1 and 8). Such CNA loci may be found using databases such as The Cancer Genome Atlas (TCGA) and the Catalogue of Somatic Mutations in Cancer (COSMIC).

In some embodiments, determining the first plurality of CNAs comprises determining quantitative measures of CNAs at each of at each of a plurality of genomic regions of the first plurality of sequencing reads, and wherein determining the second plurality of CNAs comprises determining quantitative measures of CNAs at each of at each of the plurality of genomic regions of the second plurality of sequencing reads. In some embodiments, the method further comprises correcting the first plurality of CNAs or the second plurality of CNAs for GC content and/or mappability bias. In some embodiments, the correcting comprises using a statistical modeling analysis. In some embodiments, the correcting comprises using a LOESS regression or a Bayesian model. In some embodiments, the plurality of genomic regions comprises non-overlapping genomic regions of a reference genome having a pre-determined size. In some embodiments, the pre-determined size is about 50 kilobases (kb), about 100 kb, about 200 kb, about 500 kb, about 1 megabases (Mb), about 2 Mb, about 5 Mb, or about 10 Mb.

In some embodiments, the plurality of genomic regions comprises at least about 1,000 distinct genomic regions. In some embodiments, the plurality of genomic regions comprises at least about 2,000 distinct genomic regions. In some embodiments, the plurality of genomic regions comprises at least about 3,000 distinct genomic regions, at least about 4,000 distinct genomic regions, at least about 5,000 distinct genomic regions, at least about 6,000 distinct genomic regions, at least about 7,000 distinct genomic regions, at least about 8,000 distinct genomic regions, at least about 9,000 distinct genomic regions, at least about 10,000 distinct genomic regions, at least about 15,000 distinct genomic regions, at least about 20,000 distinct genomic regions, at least about 25,000 distinct genomic regions, at least about 30,000 distinct genomic regions, at least about 35,000 distinct genomic regions, at least about 40,000 distinct genomic regions, at least about 45,000 distinct genomic regions, at least about 50,000 distinct genomic regions, at least about 100,000 distinct genomic regions, at least about 150,000 distinct genomic regions, at least about 200,000 distinct genomic regions, at least about 250,000 distinct genomic regions, at least about 300,000 distinct genomic regions, at least about 400,000 distinct genomic regions, or at least about 500,000 distinct genomic regions.

In some embodiments, determining the CNA profile change comprises processing the first plurality of CNAs and the second plurality of CNAs with a plurality of reference CNA values, wherein the plurality of reference CNA values is obtained from additional cfDNA molecules obtained or derived from additional bodily fluid samples of additional subjects. In some embodiments, the additional subjects comprise one or more subjects without cancer (e.g., subjects unaffected by cancer or subjects without a diagnosis of cancer). In some embodiments, the additional subjects comprise one or more subjects not having tumor progression. In some embodiments, the plurality of reference CNA values is obtained using additional bodily fluid samples of the subject obtained at one or more subsequent time points after the first timepoint.

In some embodiments, the method further comprises filtering out a subset of the first plurality of CNAs and the second plurality of CNAs that meet a pre-determined criterion. In some embodiments, filtering out a given CNA value of the first plurality of CNAs or the second plurality of CNAs values when the difference between the given CNA value and the corresponding reference CNA value comprises a difference of no more than about 1 standard deviation. In some embodiments, the method further comprises filtering out a given CNA value of the first plurality of CNAs or the second plurality of CNAs values when the difference between the given CNA value and the corresponding reference CNA value comprises a difference of no more than about 2 standard deviations. In some embodiments, the method further comprises filtering out a given CNA value of the first plurality of CNAs or the second plurality of CNAs values when the difference between the given CNA value and the corresponding reference CNA value comprises a difference of no more than about 3 standard deviations. In some embodiments, the method further comprises filtering out a given CNA value of the first plurality of CNAs or the second plurality of CNAs values based on a Spearman's rank correlation between the given CNA value and a corresponding local mean fragment length or a local average methylation. In some embodiments, the method further comprises filtering out a given CNA value of the first plurality of CNAs or the second plurality of CNAs values when the Spearman's rank correlation coefficient (Spearman's rho) is less than −0.1 (e.g., indicating that there is not significant negative correlation between the local mean fragment length and the local tumor copy number). This could also be done with a Pearson's correlation or some other sort of correlation statistic to ascertain whether or not there is a negative correlation between CANs and fragment length or methylation.

In some embodiments, the method further comprises normalizing the first plurality of fragment lengths or the second plurality of fragment lengths based on a library or a genomic location. In some embodiments, the method further comprises that the tumor status (e.g., tumor progression, non-progression, regression, or recurrence) comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5. In some embodiments, the method further comprises detecting a major molecular response (MMR) of the subject when the first tumor fraction or the second tumor fraction is less than 0.01, less than 0.05, less than 0.1, less than 0.2, less than 0.3, less than 0.4, or less than 0.5.

In some embodiments, the method further comprises detecting the tumor status (e.g., tumor progression, non-progression, regression, or recurrence) of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a sensitivity of at least about 95%, at least about 96%, at least about 97%, or at least about 98%. In some embodiments, the method further comprises detecting the tumor status (e.g., tumor progression, non-progression, regression, or recurrence) of the subject with a sensitivity of at least about 99%.

In some embodiments, the method further comprises detecting the tumor status (e.g., tumor progression, non-progression, regression, or recurrence) of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a specificity of at least about 95%, at least about 96%, at least about 97%, or at least about 98%. In some embodiments, the method further comprises detecting the tumor status (e.g., tumor progression, non-progression, regression, or recurrence) of the subject with a specificity of at least about 99%.

In some embodiments, the method further comprises detecting the tumor status (e.g., tumor progression, non-progression, regression, or recurrence) of the subject with a positive predictive value (PPV) of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a positive predictive value (PPV) of at least about 95%, at least about 96%, at least about 97%, or at least about 98%. In some embodiments, the method further comprises detecting the tumor status (e.g., tumor progression, non-progression, regression, or recurrence) of the subject with a positive predictive value (PPV) of at least about 99%.

In some embodiments, the method further comprises detecting the tumor status (e.g., tumor progression, non-progression, regression, or recurrence) of the subject with a negative predictive value (NPV) of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a negative predictive value (NPV) of at least about 95%, at least about 96%, at least about 97%, or at least about 98%. In some embodiments, the method further comprises detecting the tumor status (e.g., tumor progression, non-progression, regression, or recurrence) of the subject with a negative predictive value (NPV) of at least about 99%.

In some embodiments, the method further comprises detecting the tumor status (e.g., tumor progression, non-progression, regression, or recurrence) of the subject with an area under the curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, or at least about 0.94. In some embodiments, the method further comprises detecting the tumor status (e.g., tumor progression, non-progression, regression, or recurrence) of the subject with an area under the curve (AUC) of at least about 0.95, at least about 0.96, at least about 0.97, or at least about 0.98. In some embodiments, the method further comprises detecting the tumor status (e.g., tumor progression, non-progression, regression, or recurrence) of the subject with an area under the curve (AUC) of at least about 0.99.

In some embodiments, the method further comprises determining a tumor non-progression of the subject when tumor progression is not detected. In some embodiments, the method further comprises, based on the determined tumor status (e.g., tumor progression, non-progression, regression, or recurrence) of the subject, administering a therapeutically effective dose of a treatment to treat the cancer of the subject. In some embodiments, the treatment comprises treatment with surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor. In some embodiments, the detected tumor status is indicative of tumor progression, non-progression, regression, or recurrence. In some embodiments, the first and second WGS data are obtained by a sequencing device or computer processor (e.g., comprising one or more programs for executing instructions based on the methods of the present disclosure).

In another aspect, the present disclosure provides a computer system for assessing tumor progression of a subject with cancer, comprising: a database that is configured to store (i) first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject, and (ii) second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; and one or more computer processors operatively coupled to the database, wherein the one or more computer processors are individually or collectively programmed to: process the first WGS data to determine (i) a first plurality of copy number aberrations (CNAs) in the first plurality of cfDNA molecules and (ii) a first plurality of fragment lengths of the first plurality of cfDNA molecules; process the second WGS data to determine (iii) a second plurality of copy number aberrations (CNAs) in the second plurality of cfDNA molecules and (iv) a second plurality of fragment lengths of the second plurality of cfDNA molecules; process the first plurality of CNAs with the second plurality of CNAs to determine a CNA profile change; process the first plurality of fragment lengths with the second plurality of fragment lengths to determine a fragment length profile change; determine a first tumor fraction of the subject at the first timepoint or a second tumor fraction of the subject at the second timepoint, based at least in part on the CNA profile change and the fragment length profile change; and detect a tumor progression of the subject based at least in part on the first tumor fraction or the second tumor fraction.

In another aspect, the present disclosure provides a computer system for assessing tumor status (e.g., tumor progression, non-progression, regression, or recurrence) of a subject with cancer, comprising: a database that is configured to store (i) first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject, and (ii) second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; and one or more computer processors operatively coupled to the database, wherein the one or more computer processors are individually or collectively programmed to: determine, based on the first WGS data, (i) a first plurality of copy number aberrations (CNAs) in the first plurality of cfDNA molecules and (ii) a first plurality of fragment lengths of the first plurality of cfDNA molecules; determine, based on the second WGS data, (iii) a second plurality of copy number aberrations (CNAs) in the second plurality of cfDNA molecules and (iv) a second plurality of fragment lengths of the second plurality of cfDNA molecules; compare the first plurality of CNAs with the second plurality of CNAs to determine a CNA profile change; determine a fragment length profile change based on the first plurality of fragment lengths and the second plurality of fragment lengths; determine a first tumor fraction of the subject at the first timepoint or a second tumor fraction of the subject at the second timepoint, based at least in part on the CNA profile change and the fragment length profile change; and detect a tumor status of the subject based at least in part on the first tumor fraction or the second tumor fraction.

In another aspect, the present disclosure provides a non-transitory computer-readable medium comprising machine-executable instructions which, upon execution by one or more computer processors, perform a method for assessing tumor progression of a subject with cancer, the method comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; processing the first WGS data to determine (i) a first plurality of copy number aberrations (CNAs) in the first plurality of cfDNA molecules and (ii) a first plurality of fragment lengths of the first plurality of cfDNA molecules; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; processing the second WGS data to determine (iii) a second plurality of copy number aberrations (CNAs) in the second plurality of cfDNA molecules and (iv) a second plurality of fragment lengths of the second plurality of cfDNA molecules; processing the first plurality of CNAs with the second plurality of CNAs to determine a CNA profile change; processing the first plurality of fragment lengths with the second plurality of fragment lengths to determine a fragment length profile change; determining a first tumor fraction of the subject at the first timepoint or a second tumor fraction of the subject at the second timepoint, based at least in part on the CNA profile change and the fragment length profile change; and detecting a tumor progression of the subject based at least in part on the first tumor fraction or the second tumor fraction.

In another aspect, the present disclosure provides a non-transitory computer-readable medium comprising machine-executable instructions which, upon execution by one or more computer processors, perform a method for assessing tumor status (e.g., tumor progression, non-progression, regression, or recurrence) of a subject with cancer, the method comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; determining, based on the first WGS data, (i) a first plurality of copy number aberrations (CNAs) in the first plurality of cfDNA molecules and (ii) a first plurality of fragment lengths of the first plurality of cfDNA molecules; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; determining, based on the second WGS data, (iii) a second plurality of copy number aberrations (CNAs) in the second plurality of cfDNA molecules and (iv) a second plurality of fragment lengths of the second plurality of cfDNA molecules; comparing the first plurality of CNAs with the second plurality of CNAs to determine a CNA profile change; determining a fragment length profile change based on the first plurality of fragment lengths and the second plurality of fragment lengths; determining a first tumor fraction of the subject at the first timepoint or a second tumor fraction of the subject at the second timepoint, based at least in part on the CNA profile change and the fragment length profile change; and detecting a tumor status of the subject based at least in part on the first tumor fraction or the second tumor fraction.

In another aspect, the present disclosure provides a method for assessing tumor progression of a subject with cancer, comprising: obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject; processing the first MS data to determine an average methylation fraction for each of one or more CpG islands in the region of the genome, thereby obtaining a first average methylation fraction profile; obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; processing the second MS data to determine an average methylation fraction for each of one or more CpG islands in the region of the genome, thereby obtaining a second average methylation fraction profile; processing the first average methylation fraction profile across the one or more CpG islands and the second average methylation fraction profile across the one or more CpG islands to determine a methylation fraction profile; determining a first tumor fraction of the subject at the first timepoint or a second tumor fraction of the subject at the second timepoint, based at least in part on the respective methylation fraction profiles; and detecting a tumor progression of the subject based at least in part on the first tumor fraction or the second tumor fraction. In some embodiments, additional timepoints, subsequent to the second time point, may be taken and analyzed in order to detect tumor progression occurring at a later time.

In another aspect, the present disclosure provides a method for assessing tumor status (e.g., tumor progression, non-progression, regression, or recurrence) of a subject with cancer, comprising: obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject; determining, based on the first MS data, an average methylation fraction for each of one or more CpG islands in the region of the genome, thereby obtaining a first average methylation fraction profile; obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; determining, based on the second MS data, an average methylation fraction for each of one or more CpG islands in the region of the genome, thereby obtaining a second average methylation fraction profile; comparing the first average methylation fraction profile across the one or more CpG islands and the second average methylation fraction profile across the one or more CpG islands to determine a methylation fraction profile; determining a first tumor fraction of the subject at the first timepoint or a second tumor fraction of the subject at the second timepoint, based at least in part on the respective methylation fraction profiles; and detecting a tumor status of the subject based at least in part on the first tumor fraction or the second tumor fraction. In some embodiments, additional timepoints, subsequent to the second time point, may be taken and analyzed in order to detect tumor progression occurring at a later time.

In another aspect, the present disclosure provides a method for treating cancer in a subject, comprising: obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject; determining, based on the first MS data, an average methylation fraction for each of one or more CpG islands in the region of the genome, thereby obtaining a first average methylation fraction profile; obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; determining, based on the second MS data, an average methylation fraction for each of one or more CpG islands in the region of the genome, thereby obtaining a second average methylation fraction profile; comparing the first average methylation fraction profile across the one or more CpG islands and the second average methylation fraction profile across the one or more CpG islands to determine a methylation fraction profile; determining a first tumor fraction of the subject at the first timepoint or a second tumor fraction of the subject at the second timepoint, based at least in part on the respective methylation fraction profiles; detecting a tumor status of the subject based at least in part on the first tumor fraction or the second tumor fraction; and, based on the detected tumor status, administering a therapeutically effective dose of a treatment (e.g., surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor) to treat the cancer of the subject. In some embodiments, the detected tumor status comprises tumor progression, and the method comprises administering a second treatment to the patient, wherein prior to the administration, the patient has been treated with a first treatment for the cancer (and the first and second treatments are different).

In some embodiments, the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid. In some embodiments, obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads. In some embodiments, the methylation sequencing comprises whole genome bisulfite sequencing. In some embodiments, the methylation sequencing comprises whole genome enzymatic methyl-seq. In some embodiments, the methylation sequencing comprises oxidative bisulfite sequencing, TET-assisted pyridine borane sequencing (TAPS), TET-assisted bisulfite sequencing (TABS), oxidative bisulfite sequencing (oxBS-Seq), APOBEC-coupled epigenetic sequencing (ACE-seq), methylated DNA immunoprecipitation (MeDIP) sequencing, hydroxymethylated DNA immunoprecipitation (hMeDIP) sequencing, methylation array analysis, reduced representation bisulfite sequencing (RRBS-Seq), or cytosine 5-hydroxymethylation sequencing.

In some embodiments, the methylation sequencing is performed at a depth of no more than about 40×. In some embodiments, the methylation sequencing is performed at a depth of no more than about 30×. In some embodiments, the methylation sequencing is performed at a depth of no more than about 25×. In some embodiments, the methylation sequencing is performed at a depth of no more than about 20×. In some embodiments, the methylation sequencing is performed at a depth of no more than about 12×. In some embodiments, the methylation sequencing is performed at a depth of no more than about 10×. In some embodiments, the methylation sequencing is performed at a depth of no more than about 8×. In some embodiments, the methylation sequencing is performed at a depth of no more than about 6×. In some embodiments, the methylation sequencing is performed at a depth of no more than about 5×, no more than about 4×, no more than about 3×, no more than about 2×, or no more than about 1×.

In some embodiments, the method further comprises aligning the first or second plurality of sequencing reads to a reference genome (e.g., simultaneously with a C-to-T converted version of the reference genome), thereby producing a plurality of aligned sequencing reads. In some embodiments, the method further comprises enriching the first or second plurality of cfDNA molecules for the region of the genome. In some embodiments, the enrichment comprises amplifying the first or second plurality of cfDNA molecules. In some embodiments, the amplification comprises selective amplification. In some embodiments, the amplification comprises universal amplification. In some embodiments, the enrichment comprises selectively isolating at least a portion of the first or second plurality of cfDNA molecules. In some embodiments, selectively isolating the at least the portion of the first or second plurality of cfDNA molecules comprises using a plurality of probes, each of the plurality of probes having sequence complementarity with at least a portion of the region of the genome. In some embodiments, the at least the portion comprises a tumor marker locus. In some embodiments, the at least the portion comprises a plurality of tumor marker loci. In some embodiments, the plurality of tumor marker loci comprises one or more loci selected from The Cancer Genome Atlas (TCGA) or Catalogue of Somatic Mutations in cancer (COSMIC).

In some embodiments, the region of the genome comprises one or more of: CpG islands, CpG shores, patient-specific partially methylated domains, common partially methylated domains, promoters, gene bodies, evenly spaced genomewide bins, and transposable elements. In some embodiments, the region of the genome comprises a plurality of non-overlapping regions of the genome. In some embodiments, the plurality of non-overlapping regions of the genome have a pre-determined size. In some embodiments, the pre-determined size is about 50 kilobases (kb), about 100 kb, about 200 kb, about 500 kb, about 1 megabases (Mb), about 2 Mb, about 5 Mb, or about 10 Mb. In some embodiments, the region of the genome comprises one or more MAGE (melanoma-associated antigen) genes, e.g., human MAGE genes. In some embodiments, the region of the genome comprises one or more promoters corresponding to one or more MAGE (melanoma-associated antigen) genes, e.g., human MAGE genes.

In some embodiments, the plurality of non-overlapping regions of the genome comprises at least about 1,000 distinct regions. In some embodiments, the plurality of non-overlapping regions of the genome comprises at least about 2,000 distinct regions. In some embodiments, the plurality of non-overlapping regions of the genome comprises at least about 3,000 distinct regions, at least about 4,000 distinct regions, at least about 5,000 distinct regions, at least about 6,000 distinct regions, at least about 7,000 distinct regions, at least about 8,000 distinct regions, at least about 9,000 distinct regions, at least about 10,000 distinct regions, at least about 15,000 distinct regions, at least about 20,000 distinct regions, at least about 25,000 distinct regions, at least about 30,000 distinct regions, at least about 35,000 distinct regions, at least about 40,000 distinct regions, at least about 45,000 distinct regions, at least about 50,000 distinct regions, at least about 100,000 distinct regions, at least about 150,000 distinct regions, at least about 200,000 distinct regions, at least about 250,000 distinct regions, at least about 300,000 distinct regions, at least about 400,000 distinct regions, or at least about 500,000 distinct regions.

In some embodiments, determining the first or second tumor fraction comprises processing the methylation fraction profile with one or more reference methylation fraction profiles, wherein the one or more reference methylation fraction profiles are obtained from additional cfDNA molecules obtained or derived from additional bodily fluid samples of additional subjects. In some embodiments, the additional subjects comprise one or more subjects with cancer. In some embodiments, the additional subjects comprise one or more subjects without cancer. In some embodiments, the additional subjects comprise one or more subjects having tumor progression. In some embodiments, the additional subjects comprise one or more subjects not having tumor progression. In some embodiments, the one or more reference methylation fraction profiles are obtained using additional bodily fluid samples of the subject obtained at one or more subsequent time points after the first timepoint.

In some embodiments, the method further comprises detecting that the tumor status (e.g., tumor progression, non-progression, regression, or recurrence) comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5. In some embodiments, the method further comprises detecting a major molecular response (MMR) of the subject when the first tumor fraction or the second tumor fraction is less than 0.01, less than 0.05, less than 0.1, less than 0.2, less than 0.3, less than 0.4, or less than 0.5. In some embodiments, the method further comprises detecting the tumor progression of the subject when the first tumor fraction or the second tumor fraction is statistically significantly greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5. In some embodiments, the method further comprises detecting a major molecular response (MMR) of the subject when the first tumor fraction or the second tumor fraction is statistically significantly less than 0.01, less than 0.05, less than 0.1, less than 0.2, less than 0.3, less than 0.4, or less than 0.5.

In some embodiments, the method further comprises detecting the tumor status (e.g., tumor progression, non-progression, regression, or recurrence) of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a sensitivity of at least about 95%, at least about 96%, at least about 97%, or at least about 98%. In some embodiments, the method further comprises detecting the tumor status (e.g., tumor progression, non-progression, regression, or recurrence) of the subject with a sensitivity of at least about 99%.

In some embodiments, the method further comprises detecting the tumor status (e.g., tumor progression, non-progression, regression, or recurrence) of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a specificity of at least about 95%, at least about 96%, at least about 97%, or at least about 98%. In some embodiments, the method further comprises detecting the tumor status (e.g., tumor progression, non-progression, regression, or recurrence) of the subject with a specificity of at least about 99%.

In some embodiments, the method further comprises detecting the tumor status (e.g., tumor progression, non-progression, regression, or recurrence) of the subject with a positive predictive value (PPV) of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a positive predictive value (PPV) of at least about 95%, at least about 96%, at least about 97%, or at least about 98%. In some embodiments, the method further comprises detecting the tumor status (e.g., tumor progression, non-progression, regression, or recurrence) of the subject with a positive predictive value (PPV) of at least about 99%.

In some embodiments, the method further comprises detecting the tumor status (e.g., tumor progression, non-progression, regression, or recurrence) of the subject with a negative predictive value (NPV) of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a negative predictive value (NPV) of at least about 95%, at least about 96%, at least about 97%, or at least about 98%. In some embodiments, the method further comprises detecting the tumor status (e.g., tumor progression, non-progression, regression, or recurrence) of the subject with a negative predictive value (NPV) of at least about 99%.

In some embodiments, the method further comprises detecting the tumor status (e.g., tumor progression, non-progression, regression, or recurrence) of the subject with an area under the curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, or at least about 0.94. In some embodiments, the method further comprises detecting the tumor status (e.g., tumor progression, non-progression, regression, or recurrence) of the subject with an area under the curve (AUC) of at least about 0.95, at least about 0.96, at least about 0.97, or at least about 0.98. In some embodiments, the method further comprises detecting the tumor status (e.g., tumor progression, non-progression, regression, or recurrence) of the subject with an area under the curve (AUC) of at least about 0.99.

In some embodiments, the method further comprises determining a tumor non-progression of the subject when the tumor progression is not detected. In some embodiments, the method further comprises, based on the determined tumor status (e.g., tumor progression, non-progression, regression, or recurrence) of the subject, administering a therapeutically effective dose of a second therapeutic to treat the cancer of the subject. In some embodiments, the second therapeutic comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor. In some embodiments, the first and the second pluralities of cfDNA molecules are from immune cells of the subject. In some embodiments, the detected tumor status is indicative of tumor progression, non-progression, regression, or recurrence. In some embodiments, the first and second MS data are obtained by a sequencing device or computer processor (e.g., comprising one or more programs for executing instructions based on the methods of the present disclosure).

In some embodiments according to any of the embodiments described herein, the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.

In another aspect, the present disclosure provides a computer system for assessing tumor progression of a subject with cancer, comprising: a database that is configured to store (i) first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject, and (ii) second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; and one or more computer processors operatively coupled to the database, wherein the one or more computer processors are individually or collectively programmed to: process the first MS data to determine an average methylation fraction for each of one or more CpG islands in the region of the genome, thereby obtaining a first average methylation fraction profile; process the second MS data to determine an average methylation fraction for each of one or more CpG islands in the region of the genome, thereby obtaining a second average methylation fraction profile; process the first average methylation fraction profile across the one or more CpG islands and the second average methylation fraction profile across the one or more CpG islands to determine a methylation fraction profile; determine a first tumor fraction of the subject at the first timepoint or a second tumor fraction of the subject at the second timepoint, based at least in part on the respective methylation fraction profiles; and detect a tumor progression of the subject based at least in part on the first tumor fraction or the second tumor fraction.

In another aspect, the present disclosure provides a computer system for assessing tumor status (e.g., tumor progression, non-progression, regression, or recurrence) of a subject with cancer, comprising: a database that is configured to store (i) first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject, and (ii) second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; and one or more computer processors operatively coupled to the database, wherein the one or more computer processors are individually or collectively programmed to: determine, based on the first MS data, an average methylation fraction for each of one or more CpG islands in the region of the genome, thereby obtaining a first average methylation fraction profile; determine, based on the second MS data, an average methylation fraction for each of one or more CpG islands in the region of the genome, thereby obtaining a second average methylation fraction profile; compare the first average methylation fraction profile across the one or more CpG islands and the second average methylation fraction profile across the one or more CpG islands to determine a methylation fraction profile; determine a first tumor fraction of the subject at the first timepoint or a second tumor fraction of the subject at the second timepoint, based at least in part on the respective methylation fraction profiles; and detect a tumor status of the subject based at least in part on the first tumor fraction or the second tumor fraction.

In another aspect, the present disclosure provides a non-transitory computer-readable medium comprising machine-executable instructions which, upon execution by one or more computer processors, perform a method for assessing tumor progression of a subject with cancer, the method comprising: obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject; processing the first MS data to determine an average methylation fraction for each of one or more CpG islands in the region of the genome, thereby obtaining a first average methylation fraction profile; obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; processing the second MS data to determine an average methylation fraction for each of one or more CpG islands in the region of the genome, thereby obtaining a second average methylation fraction profile; processing the first average methylation fraction profile across the one or more CpG islands and the second average methylation fraction profile across the one or more CpG islands to determine a methylation fraction profile; determining a first tumor fraction of the subject at the first timepoint or a second tumor fraction of the subject at the second timepoint, based at least in part on the respective methylation fraction profiles; and detecting a tumor progression of the subject based at least in part on the first tumor fraction or the second tumor fraction.

In another aspect, the present disclosure provides a non-transitory computer-readable medium comprising machine-executable instructions which, upon execution by one or more computer processors, perform a method for assessing tumor status (e.g., tumor progression, non-progression, regression, or recurrence) of a subject with cancer, the method comprising: obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject; determining, based on the first MS data, an average methylation fraction for each of one or more CpG islands in the region of the genome, thereby obtaining a first average methylation fraction profile; obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; determining, based on the second MS data, an average methylation fraction for each of one or more CpG islands in the region of the genome, thereby obtaining a second average methylation fraction profile; comparing the first average methylation fraction profile across the one or more CpG islands and the second average methylation fraction profile across the one or more CpG islands to determine a methylation fraction profile; determining a first tumor fraction of the subject at the first timepoint or a second tumor fraction of the subject at the second timepoint, based at least in part on the respective methylation fraction profiles; and detecting a tumor status of the subject based at least in part on the first tumor fraction or the second tumor fraction.

In some embodiments, the detected tumor progression is based at least in part on one or more statistical modeling analyses of the respective methylation fraction profiles. In some embodiments, the one or more statistical modeling analyses comprise linear regression, simple regression, binary regression, Bayesian linear regression, Bayesian modeling, polynomial regression, Gaussian process regression, Gaussian modeling, binary regression, logistic regression, or nonlinear regression. In some embodiments, the one or more statistical modeling analyses compare the detected tumor progression with MS data derived from a sample having a known tumor fraction, MS data derived from a pure tumor sample, or MS data derived from a healthy sample.

In another aspect, the present disclosure provides a method for assessing tumor status (e.g., tumor progression, non-progression, regression, or recurrence) of a subject with cancer, comprising: obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject; determining, based on the first MS data, a methylation profile for each of one or more loci of the genome (e.g., one or more CpG islands or non-CpG methylated loci in a genomic region), thereby obtaining a first methylation profile; obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; determining, based on the second MS data, a methylation profile for each of one or more loci of the genome (e.g., one or more CpG islands or non-CpG methylated loci in a genomic region), thereby obtaining a second methylation profile; comparing the first methylation profile across the one or more loci and the second methylation profile across the one or more loci; determining a first tumor fraction of the subject at the first timepoint or a second tumor fraction of the subject at the second timepoint, based at least in part on the respective methylation fraction profiles; and detecting a tumor status of the subject based at least in part on the first tumor fraction or the second tumor fraction. In some embodiments, additional timepoints, subsequent to the second time point, may be taken and analyzed in order to detect tumor progression occurring at a later time.

In another aspect, the present disclosure provides a method for treating cancer in a subject, comprising: obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject; determining, based on the first MS data, a methylation profile for each of one or more loci of the genome (e.g., one or more CpG islands or non-CpG methylated loci in a genomic region), thereby obtaining a first methylation profile; obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; determining, based on the second MS data, a methylation profile for each of one or more loci of the genome (e.g., one or more CpG islands or non-CpG methylated loci in a genomic region), thereby obtaining a second methylation profile; comparing the first methylation profile across the one or more loci and the second methylation profile across the one or more loci; determining a first tumor fraction of the subject at the first timepoint or a second tumor fraction of the subject at the second timepoint, based at least in part on the respective methylation fraction profiles; detecting a tumor status of the subject based at least in part on the first tumor fraction or the second tumor fraction; and, based on the detected tumor status, administering a therapeutically effective dose of a treatment (e.g., surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor) to treat the cancer of the subject. In some embodiments, the detected tumor status comprises tumor progression, and the method comprises administering a second treatment to the patient, wherein prior to the administration, the patient has been treated with a first treatment for the cancer (and the first and second treatments are different).

In another aspect, the present disclosure provides a method for assessing tumor status (e.g., tumor progression, non-progression, regression, or recurrence) of a subject with cancer, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; determining, based on the first WGS data, (i) a first plurality of copy number aberrations (CNAs) in the first plurality of cfDNA molecules and (ii) a first plurality of fragment lengths of the first plurality of cfDNA molecules; obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint; determining, based on the first MS data, an average methylation fraction for each of one or more CpG islands in the region of the genome, thereby obtaining a first average methylation fraction profile; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; determining, based on the second WGS data, (iii) a second plurality of copy number aberrations (CNAs) in the second plurality of cfDNA molecules and (iv) a second plurality of fragment lengths of the second plurality of cfDNA molecules; obtaining second MS data of a second plurality of cell-free DNA (cfDNA) molecules across the region of the genome, wherein the second plurality of cfDNA molecules is obtained or derived from a bodily fluid sample of the subject at the second timepoint; determining, based on the second MS data, an average methylation fraction for each of one or more CpG islands in the region of the genome, thereby obtaining a second average methylation fraction profile; comparing the first plurality of CNAs with the second plurality of CNAs to determine a CNA profile change; determining a fragment length profile change based on the first plurality of fragment lengths and the second plurality of fragment lengths; comparing the first average methylation fraction profile across the one or more CpG islands and the second average methylation fraction profile across the one or more CpG islands to determine a methylation fraction profile; determining a first tumor fraction of the subject at the first timepoint or a second tumor fraction of the subject at the second timepoint, based at least in part on the CNA profile change, the fragment length profile change, and the respective methylation fraction profiles; and detecting a tumor status of the subject based at least in part on the first tumor fraction or the second tumor fraction.

In another aspect, the present disclosure provides a method for treating cancer in a subject, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; determining, based on the first WGS data, (i) a first plurality of copy number aberrations (CNAs) in the first plurality of cfDNA molecules and (ii) a first plurality of fragment lengths of the first plurality of cfDNA molecules; obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint; determining, based on the first MS data, an average methylation fraction for each of one or more CpG islands in the region of the genome, thereby obtaining a first average methylation fraction profile; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; determining, based on the second WGS data, (iii) a second plurality of copy number aberrations (CNAs) in the second plurality of cfDNA molecules and (iv) a second plurality of fragment lengths of the second plurality of cfDNA molecules; obtaining second MS data of a second plurality of cell-free DNA (cfDNA) molecules across the region of the genome, wherein the second plurality of cfDNA molecules is obtained or derived from a bodily fluid sample of the subject at the second timepoint; determining, based on the second MS data, an average methylation fraction for each of one or more CpG islands in the region of the genome, thereby obtaining a second average methylation fraction profile; comparing the first plurality of CNAs with the second plurality of CNAs to determine a CNA profile change; determining a fragment length profile change based on the first plurality of fragment lengths and the second plurality of fragment lengths; comparing the first average methylation fraction profile across the one or more CpG islands and the second average methylation fraction profile across the one or more CpG islands to determine a methylation fraction profile; determining a first tumor fraction of the subject at the first timepoint or a second tumor fraction of the subject at the second timepoint, based at least in part on the CNA profile change, the fragment length profile change, and the respective methylation fraction profiles; detecting a tumor status of the subject based at least in part on the first tumor fraction or the second tumor fraction; and, based on the detected tumor status, administering a therapeutically effective dose of a treatment (e.g., surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor) to treat the cancer of the subject. In some embodiments, the detected tumor status comprises tumor progression, and the method comprises administering a second treatment to the patient, wherein prior to the administration, the patient has been treated with a first treatment for the cancer (and the first and second treatments are different).

In another aspect, the present disclosure provides a method for assessing tumor status (e.g., tumor progression, non-progression, regression, or recurrence) of a subject with cancer, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; determining, based on the first WGS data, (i) a first plurality of copy number aberrations (CNAs) in the first plurality of cfDNA molecules and (ii) a first plurality of fragment lengths of the first plurality of cfDNA molecules; obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint; determining, based on the first MS data, a methylation profile for each of one or more loci of the genome, thereby obtaining a first methylation profile; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; determining, based on the second WGS data, (iii) a second plurality of copy number aberrations (CNAs) in the second plurality of cfDNA molecules and (iv) a second plurality of fragment lengths of the second plurality of cfDNA molecules; obtaining second MS data of a second plurality of cell-free DNA (cfDNA) molecules across the region of the genome, wherein the second plurality of cfDNA molecules is obtained or derived from a bodily fluid sample of the subject at the second timepoint; determining, based on the second MS data, a methylation profile for each of one or more loci of the genome, thereby obtaining a second methylation profile; comparing the first plurality of CNAs with the second plurality of CNAs to determine a CNA profile change; determining a fragment length profile change based on the first plurality of fragment lengths and the second plurality of fragment lengths; comparing the first methylation profile across the one or more loci and the second methylation profile across the one or more loci; determining a first tumor fraction of the subject at the first timepoint or a second tumor fraction of the subject at the second timepoint, based at least in part on the CNA profile change, the fragment length profile change, and the respective methylation fraction profiles; and detecting a tumor status of the subject based at least in part on the first tumor fraction or the second tumor fraction.

In another aspect, the present disclosure provides a method for treating cancer in a subject, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; determining, based on the first WGS data, (i) a first plurality of copy number aberrations (CNAs) in the first plurality of cfDNA molecules and (ii) a first plurality of fragment lengths of the first plurality of cfDNA molecules; obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint; determining, based on the first MS data, a methylation profile for each of one or more loci of the genome, thereby obtaining a first methylation profile; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; determining, based on the second WGS data, (iii) a second plurality of copy number aberrations (CNAs) in the second plurality of cfDNA molecules and (iv) a second plurality of fragment lengths of the second plurality of cfDNA molecules; obtaining second MS data of a second plurality of cell-free DNA (cfDNA) molecules across the region of the genome, wherein the second plurality of cfDNA molecules is obtained or derived from a bodily fluid sample of the subject at the second timepoint; determining, based on the second MS data, a methylation profile for each of one or more loci of the genome, thereby obtaining a second methylation profile; comparing the first plurality of CNAs with the second plurality of CNAs to determine a CNA profile change; determining a fragment length profile change based on the first plurality of fragment lengths and the second plurality of fragment lengths; comparing the first methylation profile across the one or more loci and the second methylation profile across the one or more loci; determining a first tumor fraction of the subject at the first timepoint or a second tumor fraction of the subject at the second timepoint, based at least in part on the CNA profile change, the fragment length profile change, and the respective methylation fraction profiles; detecting a tumor status of the subject based at least in part on the first tumor fraction or the second tumor fraction; and, based on the detected tumor status, administering a therapeutically effective dose of a treatment (e.g., surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor) to treat the cancer of the subject. In some embodiments, the detected tumor status comprises tumor progression, and the method comprises administering a second treatment to the patient, wherein prior to the administration, the patient has been treated with a first treatment for the cancer (and the first and second treatments are different).

In some embodiments, the first and the second methylation profiles comprise 5-hydroxymethylcytosine status, 5-methylcytosine status, enrichment-based methylation assessment, median methylation level, mode methylation level, maximum methylation level, or minimum methylation level. In some embodiments, the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid. In some embodiments, obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads. In some embodiments, the methylation sequencing comprises whole genome bisulfite sequencing. In some embodiments, the methylation sequencing comprises whole genome enzymatic methyl-seq. In some embodiments, the methylation sequencing comprises oxidative bisulfite sequencing, TET-assisted pyridine borane sequencing (TAPS), TET-assisted bisulfite sequencing (TABS), oxidative bisulfite sequencing (oxBS-Seq), APOBEC-coupled epigenetic sequencing (ACE-seq), methylated DNA immunoprecipitation (MeDIP) sequencing, hydroxymethylated DNA immunoprecipitation (hMeDIP) sequencing, methylation array analysis, reduced representation bisulfite sequencing (RRBS-Seq), or cytosine 5-hydroxymethylation sequencing.

In some embodiments, the methylation sequencing is performed at a depth of no more than about 40×. In some embodiments, the methylation sequencing is performed at a depth of no more than about 30×. In some embodiments, the methylation sequencing is performed at a depth of no more than about 25×. In some embodiments, the methylation sequencing is performed at a depth of no more than about 20×. In some embodiments, the methylation sequencing is performed at a depth of no more than about 12×. In some embodiments, the methylation sequencing is performed at a depth of no more than about 10×. In some embodiments, the methylation sequencing is performed at a depth of no more than about 8×. In some embodiments, the methylation sequencing is performed at a depth of no more than about 6×. In some embodiments, the methylation sequencing is performed at a depth of no more than about 5×, no more than about 4×, no more than about 3×, no more than about 2×, or no more than about 1×.

In some embodiments, the method further comprises aligning the first or second plurality of sequencing reads to a reference genome (e.g., simultaneously with a C-to-T converted version of the reference genome), thereby producing a plurality of aligned sequencing reads. In some embodiments, the method further comprises enriching the first or second plurality of cfDNA molecules for the region of the genome. In some embodiments, the enrichment comprises amplifying the first or second plurality of cfDNA molecules. In some embodiments, the amplification comprises selective amplification. In some embodiments, the amplification comprises universal amplification. In some embodiments, the enrichment comprises selectively isolating at least a portion of the first or second plurality of cfDNA molecules. In some embodiments, selectively isolating the at least the portion of the first or second plurality of cfDNA molecules comprises using a plurality of probes, each of the plurality of probes having sequence complementarity with at least a portion of the region of the genome. In some embodiments, the at least the portion comprises a tumor marker locus. In some embodiments, the at least the portion comprises a plurality of tumor marker loci. In some embodiments, the plurality of tumor marker loci comprises one or more loci selected from The Cancer Genome Atlas (TCGA) or Catalogue of Somatic Mutations in cancer (COSMIC).

In some embodiments, the loci or region(s) of the genome comprise one or more of: CpG islands, CpG shores, patient-specific partially methylated domains, common partially methylated domains, promoters, gene bodies, evenly spaced genomewide bins, and transposable elements. In some embodiments, the region of the genome comprises a plurality of non-overlapping regions of the genome. In some embodiments, the plurality of non-overlapping regions of the genome have a pre-determined size. In some embodiments, the pre-determined size is about 50 kilobases (kb), about 100 kb, about 200 kb, about 500 kb, about 1 megabases (Mb), about 2 Mb, about 5 Mb, or about 10 Mb.

In some embodiments, the plurality of non-overlapping regions of the genome comprises at least about 1,000 distinct regions. In some embodiments, the plurality of non-overlapping regions of the genome comprises at least about 2,000 distinct regions. In some embodiments, the plurality of non-overlapping regions of the genome comprises at least about 3,000 distinct regions, at least about 4,000 distinct regions, at least about 5,000 distinct regions, at least about 6,000 distinct regions, at least about 7,000 distinct regions, at least about 8,000 distinct regions, at least about 9,000 distinct regions, at least about 10,000 distinct regions, at least about 15,000 distinct regions, at least about 20,000 distinct regions, at least about 25,000 distinct regions, at least about 30,000 distinct regions, at least about 35,000 distinct regions, at least about 40,000 distinct regions, at least about 45,000 distinct regions, at least about 50,000 distinct regions, at least about 100,000 distinct regions, at least about 150,000 distinct regions, at least about 200,000 distinct regions, at least about 250,000 distinct regions, at least about 300,000 distinct regions, at least about 400,000 distinct regions, or at least about 500,000 distinct regions.

In some embodiments, determining the first or second tumor fraction comprises processing the methylation fraction profile with one or more reference methylation fraction profiles, wherein the one or more reference methylation fraction profiles are obtained from additional cfDNA molecules obtained or derived from additional bodily fluid samples of additional subjects. In some embodiments, the additional subjects comprise one or more subjects with cancer. In some embodiments, the additional subjects comprise one or more subjects without cancer. In some embodiments, the additional subjects comprise one or more subjects having tumor progression. In some embodiments, the additional subjects comprise one or more subjects not having tumor progression. In some embodiments, the one or more reference methylation fraction profiles are obtained using additional bodily fluid samples of the subject obtained at one or more subsequent time points after the first timepoint.

In some embodiments, the method further comprises detecting that the tumor status (e.g., tumor progression, non-progression, regression, or recurrence) comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5. In some embodiments, the method further comprises detecting a major molecular response (MMR) of the subject when the first tumor fraction or the second tumor fraction is less than 0.01, less than 0.05, less than 0.1, less than 0.2, less than 0.3, less than 0.4, or less than 0.5. In some embodiments, the method further comprises detecting that the tumor status (e.g., tumor progression, non-progression, regression, or recurrence) comprises tumor progression of the subject when the first tumor fraction or the second tumor fraction is statistically significantly greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5. In some embodiments, the method further comprises detecting a major molecular response (MMR) of the subject when the first tumor fraction or the second tumor fraction is statistically significantly less than 0.01, less than 0.05, less than 0.1, less than 0.2, less than 0.3, less than 0.4, or less than 0.5.

In some embodiments, the method further comprises detecting the tumor status (e.g., tumor progression, non-progression, regression, or recurrence) of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a sensitivity of at least about 95%, at least about 96%, at least about 97%, or at least about 98%. In some embodiments, the method further comprises detecting the tumor status (e.g., tumor progression, non-progression, regression, or recurrence) of the subject with a sensitivity of at least about 99%.

In some embodiments, the method further comprises detecting the tumor status (e.g., tumor progression, non-progression, regression, or recurrence) of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a specificity of at least about 95%, at least about 96%, at least about 97%, or at least about 98%. In some embodiments, the method further comprises detecting the tumor status (e.g., tumor progression, non-progression, regression, or recurrence) of the subject with a specificity of at least about 99%.

In some embodiments, the method further comprises detecting the tumor status (e.g., tumor progression, non-progression, regression, or recurrence) of the subject with a positive predictive value (PPV) of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a positive predictive value (PPV) of at least about 95%, at least about 96%, at least about 97%, or at least about 98%. In some embodiments, the method further comprises detecting the tumor status (e.g., tumor progression, non-progression, regression, or recurrence) of the subject with a positive predictive value (PPV) of at least about 99%.

In some embodiments, the method further comprises detecting the tumor status (e.g., tumor progression, non-progression, regression, or recurrence) of the subject with a negative predictive value (NPV) of at least about 50%, at least about 55%, at least about 60%, at least about 65%, at least about 70%, at least about 75%, at least about 80%, at least about 85%, at least about 90%, at least about 91%, at least about 92%, at least about 93%, or at least about 94%. In some embodiments, the method further comprises detecting the tumor status (e.g., tumor progression, non-progression, regression, or recurrence) of the subject with a negative predictive value (NPV) of at least about 95%, at least about 96%, at least about 97%, or at least about 98%. In some embodiments, the method further comprises detecting the tumor status (e.g., tumor progression, non-progression, regression, or recurrence) of the subject with a negative predictive value (NPV) of at least about 99%.

In some embodiments, the method further comprises detecting the tumor status (e.g., tumor progression, non-progression, regression, or recurrence) of the subject with an area under the curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, or at least about 0.94. In some embodiments, the method further comprises detecting the tumor status (e.g., tumor progression, non-progression, regression, or recurrence) of the subject with an area under the curve (AUC) of at least about 0.95, at least about 0.96, at least about 0.97, or at least about 0.98. In some embodiments, the method further comprises detecting the tumor status (e.g., tumor progression, non-progression, regression, or recurrence) of the subject with an area under the curve (AUC) of at least about 0.99.

In some embodiments, the method further comprises determining a tumor non-progression of the subject when tumor progression is not detected. In some embodiments, the method further comprises, based on the determined tumor status (e.g., tumor progression, non-progression, regression, or recurrence) of the subject, administering a therapeutically effective dose of a second therapeutic to treat the cancer of the subject. In some embodiments, the second therapeutic comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor. In some embodiments, the first and the second pluralities of cfDNA molecules are from immune cells of the subject. In some embodiments, the detected tumor status is indicative of tumor progression, non-progression, regression, or recurrence. In some embodiments, the first and second MS data are obtained by a sequencing device or computer processor (e.g., comprising one or more programs for executing instructions based on the methods of the present disclosure).

In some embodiments according to any of the embodiments described herein, the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example method of assessing tumor progression in a subject using a Change in Deviation (CID) score, in accordance with some embodiments.

FIG. 2 illustrates a computer system that is programmed or otherwise configured to implement methods provided herein.

FIGS. 3A-3B show an overview of the clinical setting, in accordance with some embodiments. FIG. 3A shows a diagram comparing radiographic response assessment and the potential use of cfDNA to assess molecular response. FIG. 3B shows timing of imaging and blood collections for patients in the study.

FIGS. 4A-4E show serial assessment of ctDNA to determine molecular progression, in accordance with some embodiments. FIG. 4A shows the genome-wide plots of CNAs detected for patient LS030178. The TO baseline blood draw was collected 13 days before the start of treatment, and T1 was collected 21 days after the start of treatment. FIG. 4B shows that normalized fragment length exhibit the reverse pattern compared to CNAs. FIG. 4C shows that overall there was a strong negative correlation between the normalized fragment length at each genomic position and the inferred copy number (Spearman's rho=−0.57, P <1E-10). FIG. 4D shows that patient LS030178 had an increase in TFR at follow-up time points T1 and T2, detectable in advance of imaging that indicated progressive disease. FIG. 4E shows that patient LS030093, who responded to therapy, showed a marked decrease in TFR at T1 and T2, concordant with later imaging that showed a partial response.

FIGS. 5A-5C show ctDNA assessments following first or second cycle of therapy predicted progression, in accordance with some embodiments. FIG. 5A shows a comparison of imaging results at first FUI (SLD assessed by RECIST 1.1) with ctDNA assessment of molecular progression, indicated by a confident increase in TFR for either post-treatment sample (sensitivity =54%, specificity=100%, PPV=100%, NPV=85%). Footnoted cases showed clear clinical progression. FIG. 5B shows TFR for progressors and non-progressors at T1 (left) and T2 (right), compared to radiographic or clinical assessment of PD or non-PD, showing predictive performance at each time point. FIG. 5C shows that for patients with molecular progression, detection of the molecular progression preceded the date of detection of progression by standard of care imaging by a median of 40 days (range of -21 to 103 days).

FIGS. 6A-6I show molecular response assessment early in the course of therapy was associated with favorable PFS, in accordance with some embodiments. FIG. 6A shows that the full cohort (n=92) had median PFS of 211 days. FIG. 6B shows that patients with a molecular progression detected from cfDNA at T1 or T2 (n=14, median PFS=62 days) had significantly worse PFS compared to patients with no molecular progression (n=78, median PFS=263 days; HR=12.6 [95% CI: 5.8 to 27.3]; log-rank P <1E-10). FIGS. 6C-6D show subset analysis based on therapeutic modality for patients on immunotherapy with or without chemotherapy (n=34; log-rank P=2E-12) (FIG. 6C), patients on chemotherapy with or without targeted therapy (n=42; log-rank P=7E-6) (FIG. 6D). FIGS. 6E-6F show subset analysis based on cancer type for lung cancer patients (n=40; log-rank P=8E-8) (FIG. 6E) and breast cancer patients (n=25; log-rank P=3E-4) (FIG. 6F). FIG. 6G shows that patients with a MMR had significantly longer PFS after accounting for predictions based on molecular progression (Cox P=0.011). FIGS. 6H-6I show subset analysis for patients with either stable disease or partial response determined by radiography at first FIJI (n=65) stratified by response status (log-rank P=0.4) (FIG. 6H) or MMR (log-rank P=0.02) (FIG. 61).

FIGS. 7A-7B show that methylation may provide an orthogonal signal to CNAs for response monitoring, in accordance with some embodiments. These figures show a distribution of average methylation levels in genome-wide 1 megabase bins for patient LS030083 (FIG. 7A) and LS030078 (FIG. 7B) at baseline (black line) and either T1 or T2 (orange line).

FIG. 8 shows longitudinal WGS data for a healthy individual, in accordance with some embodiments. This figure includes genome-wide plots showing no CNAs detected for participant LB-S00129 at an initial blood draw (top) and 34 days later (bottom), as in FIG. 4A.

FIG. 9 shows a comparison of tumor fraction ratio across sequencing protocols, in accordance with some embodiments. This figure shows results for 20 post-treatment samples from 13 participants that were processed with both WGS and WGBS. Two samples from patients with PD at first FUI had discordant classifications of molecular progression, with measurements of TFR that were close to the call boundary.

FIG. 10 shows sample timing and sensitivity, in accordance with some embodiments. This figure shows molecular progression and blood sample timing for the 42 samples from 26 participants with PD at first FUI (two-sample Kolmogorov—Smirnov test, P=0.15).

FIG. 11 shows molecular response assessment and PFS for other cancers, in accordance with some embodiments. This figure shows non-lung non-breast cancers (n=27; log-rank P=5E-6), plotted as in FIGS. 6E-6F.

FIGS. 12A-12B show MMR and PFS for patients with non-PD at first FUI, in accordance with some embodiments. These figures show results from all patients with radiographic partial response (n=30) (FIG. 12A) or stable disease (n=35) (FIG. 12B).

FIGS. 13A-13B show examples of Kaplan-Meier progression free survival (PFS) and overall survival (OS) plots for each of these three patient categories (MP, MMR, and neither MP nor MMR) in the patient cohort, in accordance with some embodiments. These figures show that the survival curves are highly separated from each other. Furthermore, the predictions of molecular progression predict radiographic progression with high specificity.

FIGS. 14A-14C show examples of a strong average decrease in methylation observed at three MAGE genes (MAGEA1, MAGEA3, and MAGEA4), in accordance with some embodiments.

FIGS. 15A & 15B show that quantifying the change in strength of a specific copy number aberrations (CNA) in multiple samples from a patient over the course of treatment is less prone to certain error modes arising from separately quantifying tumor fractions in separate samples based on CNAs.

### DETAILED DESCRIPTION

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (P03) groups. A nucleotide can include a nucleobase, a five-carbon sugar (either ribose or deoxyribose), and one or more phosphate groups, individually or in combination.

Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate (dNTP), which can be selected from deoxyadenosine triphosphate (dATP), deoxycytosine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a nucleic acid is deoxyribonucleic acid (DNA), ribonucleic acid (RNA), or derivatives or variants thereof. A nucleic acid may be single-stranded or double stranded. A nucleic acid molecule may be linear, curved, or circular or any combination thereof.

The terms “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or ribonucleotides (RNA), or analogs thereof. A nucleic acid molecule can have a length of at least about 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb or it may have any number of bases between any two of the aforementioned values. An oligonucleotide is typically composed of a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The term “sample,” as used herein, generally refers to a biological sample. Examples of biological samples include nucleic acid molecules, amino acids, polypeptides, proteins, carbohydrates, fats, or viruses. In an example, a biological sample is a nucleic acid sample including one or more nucleic acid molecules. The nucleic acid molecules may be cell-free or cell-free nucleic acid molecules, such as cell-free DNA (cfDNA) or cell-free RNA (cfRNA). The nucleic acid molecules may be derived from a variety of sources including human, mammal, non-human mammal, ape, monkey, chimpanzee, reptilian, amphibian, or avian, sources. Further, samples may be extracted from a variety of animal fluids containing cell-free sequences, including but not limited to bodily fluid samples such as blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, lymph fluid, and the like. Cell free polynucleotides (e.g., cfDNA) may be fetal in origin (via fluid taken from a pregnant subject), or may be derived from tissue of the subject itself.

The term “subject,” as used herein, generally refers to an individual having a biological sample that is undergoing processing or analysis. A subject can be an animal or plant. The subject can be a mammal, such as a human, dog, cat, horse, pig or rodent. The subject can be a patient, e.g., have or be suspected of having a disease, such as one or more cancers (e.g., brain cancer, breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, urinary tract cancer), one or more infectious diseases, one or more genetic disorder, or one or more tumors, or any combination thereof. For subjects having or suspected of having one or more tumors, the tumors may be of one or more types.

The term “whole blood,” as used herein, generally refers to a blood sample that has not been separated into sub-components (e.g., by centrifugation). The whole blood of a blood sample may contain cfDNA and/or germline DNA. Whole blood DNA (which may contain cfDNA and/or germline DNA) may be extracted from a blood sample. Whole blood DNA sequencing reads (which may contain cfDNA sequencing reads and/or germline DNA sequencing reads) may be extracted from whole blood DNA.

Assessing Tumor Progression in Cell-Free DNA Sequence Data from a Subject

Assessment of tumor progression may be relatively straightforward when a significant portion (e.g., >80%) of a sample taken from a subject comes from or is derived from tumor cells. However, in a cell free DNA (cfDNA) preparation from a subject's plasma derived from a blood sample, the detection of tumor DNA from the cfDNA and the assessment of tumor progression therefrom may be an insensitive and noisy process. Detection of tumor DNA and assessment of tumor progression from such insensitive and/or noisy signals may be challenging due to the overwhelming signal from non-tumor DNA (e.g., from germline DNA from germline cells that are not tumor derived). The present disclosure provides methods and systems for assessing tumor progression from cell-free DNA (cfDNA) sequence data (e.g., cfDNA sequencing reads) of cfDNA molecules obtained or derived from a sample of a subject (e.g., a patient with cancer). Once cfDNA sequence data has been received from analysis of a sample from the subject, one or more bioinformatics processes may be used to assess tumor progression or tumor non-progression of the subject. In some embodiments, immune cell DNA is detected from cfDNA, which can optionally be used to assess tumor progression.

In an aspect, the present disclosure provides a method for assessing tumor progression of a subject with cancer, comprising: obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject; processing the first WGS data to determine (i) a first plurality of copy number aberrations (CNAs) in the first plurality of cfDNA molecules and (ii) a first plurality of fragment lengths of the first plurality of cfDNA molecules; obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; processing the second WGS data to determine (iii) a second plurality of copy number aberrations (CNAs) in the second plurality of cfDNA molecules and (iv) a second plurality of fragment lengths of the second plurality of cfDNA molecules; processing the first plurality of CNAs with the second plurality of CNAs to determine a CNA profile change; processing the first plurality of fragment lengths with the second plurality of fragment lengths to determine a fragment length profile change; determining a first tumor fraction of the subject at the first timepoint or a second tumor fraction of the subject at the second timepoint, based at least in part on the CNA profile change and the fragment length profile change; and detecting a tumor progression of the subject based at least in part on the first tumor fraction or the second tumor fraction.

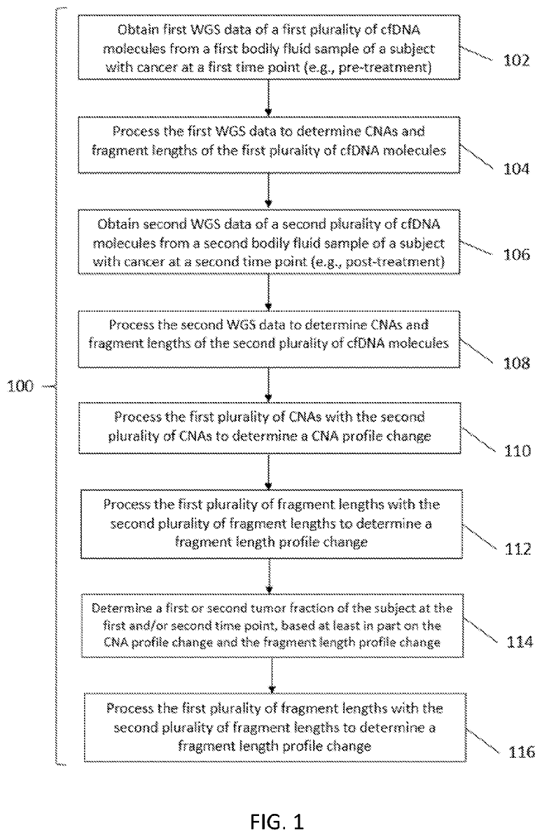

FIG. 1 illustrates an example method of assessing tumor progression in a subject, in accordance with some embodiments. In operation 102, a first whole genome sequencing (WGS) data of a first plurality of cell-free DNA (cfDNA) molecules is obtained. The first plurality of cfDNA molecules may be obtained or derived from a first bodily fluid sample of the subject at a first timepoint. The first timepoint may precede a therapeutic configured to treat the cancer being administered to the subject. In operation 104, the first WGS data is processed to determine (i) a first plurality of copy number aberrations (CNAs) in the first plurality of cfDNA molecules and (ii) a first plurality of fragment lengths of the first plurality of cfDNA molecules. In operation 106, a second whole genome sequencing (WGS) data of a second plurality of cell-free DNA (cfDNA) molecules is obtained. The second plurality of cfDNA molecules may be obtained or derived from a second bodily fluid sample of the subject at a second timepoint. The second timepoint may be after a therapeutic configured to treat the cancer is administered to the subject. In operation 108, the second WGS data is processed to determine (i) a second plurality of copy number aberrations (CNAs) in the second plurality of cfDNA molecules and (ii) a second plurality of fragment lengths of the second plurality of cfDNA molecules. In operation 110, the first plurality of CNAs is processed (e.g., compared) with the second plurality of CNAs to determine a CNA profile change. In operation 112, the first plurality of fragment lengths is processed (e.g., compared) with the second plurality of fragment lengths to determine a fragment length profile change. In operation 114, a first tumor fraction of the subject at the first timepoint and/or a second tumor fraction of the subject at the second timepoint is determined, based at least in part on the CNA profile change and the fragment length profile change. In operation 116, a tumor progression of the subject is detected, based at least in part on the first tumor fraction and or the second tumor fraction.

In some embodiments, the methods comprise identifying one or more libraries (e.g., in which tumor fraction can be determined, such as by using CNA pattern, or based on the fact that the library is from a control sample). For each library, methylation status (e.g., average methylation fraction) can be calculated from one or more regions of the genome (e.g., one, some, or all CpG islands, promoters, etc.) using methylation sequencing as described herein. Statistical modeling (e.g., linear regression or another technique of the present disclosure) can be used to regularize known tumor fraction against methylation patterns, e.g., using leave-one-participant-out cross validation. Without wishing to be bound to theory, it is thought that these methods allow the prediction of tumor fraction of a sample based on methylation patterns, e.g., even when CNA is not detectable. In some embodiments, the methods further comprise comparing the above analyses across two or more timepoints.

For example, sequencing reads may be generated from the cfDNA using any suitable sequencing method known to one of skill in the art. The sequencing method can be a first-generation sequencing method, such as Maxam-Gilbert or Sanger sequencing, or a high-throughput sequencing (e.g., next-generation sequencing or NGS) method. A high-throughput sequencing method may sequence simultaneously (or substantially simultaneously) at least 10,000, 100,000, 1 million, 10 million, 100 million, 1 billion, or more polynucleotide molecules. Sequencing methods may include, but are not limited to: pyrosequencing, sequencing-by-synthesis, single-molecule sequencing, nanopore sequencing, semiconductor sequencing, sequencing-by-ligation, sequencing-by-hybridization, Digital Gene Expression (Helicos®), massively parallel sequencing, e.g., Helicos®, Clonal Single Molecule Array (Solexa®/Illumina®), sequencing using PacBio®, SOLiD®, Ion Torrent®, or Nanopore® platforms. In some embodiments, the sequencing is by Nanopore sequencing, chain termination (Sanger) sequencing, sequencing by synthesis (e.g., Illumina or Solexa sequencing), single molecule real-time sequencing, massively parallel signature sequencing, Polony sequencing, 454 pyrosequencing, combinatorial probe anchor synthesis, sequencing by ligation (SOLiD sequencing), or GenapSys sequencing. In some embodiments, the sequencing includes hybrid capture-based sequencing (hybrid capture-based NGS), e.g., using adaptor ligation-based libraries. See, e.g., Frampton, G. M. et al. (2013) Nat. Biotech. 31:1023-1031.

In some embodiments, the sequencing method comprises bisulfite sequencing. Bisulfite sequencing typically comprises treatment of DNA with bisulfite prior to sequencing, which converts unmethylated cytosines to uracil without converting 5-methylcytosines, thereby allowing for detection of DNA methylation status (though additional methods are needed to distinguish between 5-methylcytosine and 5-hydroxymethylcytosine, as noted below). A variety of standard sequencing methods may be used after bisulfite treatment, including methods that are either specific or non-specific to detection of methylation. Sequencing methods can include, without limitation, pyrosequencing, direct sequencing (e.g., using PCR), high resolution melting analysis, methylation-sensitive single-strand conformation analysis, methylation-sensitive single-nucleotide primer extension, base-specific cleavage/MALDI-TOF, sequence analysis by microarray, and methylation-specific PCR.

In some embodiments, the sequencing method comprises oxidative bisulfite sequencing. Oxidative bisulfite sequencing can be used to distinguish between 5-methylcytosine and 5-hydroxymethylcytosine by chemical oxidation of 5-hydroxymethylcytosine to 5-formylcytosine, which can be converted to uracil via bisulfite treatment.

In some embodiments, the sequencing method comprises TET based methylation sequencing, such as TET-assisted pyridine borane sequencing (TAPS) or TET-assisted bisulfite sequencing (TABS or TAB-Seq). TAB-Seq allows for resolution of 5-hydroxymethylcytosine by using ten-eleven translocation (TET) dioxygenase enzymes. In an exemplary method, β-glucosyltransferase (βGT) is used to convert 5-hydroxymethylcytosine into β-glucosyl-5-hydroxymethylcytosine (which blocks further modification by TET and oxidation by bisulfite), and TET enzyme is used to oxidize 5-hydroxymethylcytosine to 5-carboxylcytosine, which is sensitive to uracil conversion via bisulfite. See, e.g., Yu, M. et al. (2012) Cell 149:1368-1380. For TAPS, TET enzyme is used to oxidize 5-methylcytosine and 5-hydroxymethylcytosine to 5-carboxylcytosine, then pyridine borane reduction converts 5-carboxylcytosine to dihydrouracil (DHU), which can be read as thymine via PCR. See, e.g., Liu, Y. et al. (2019) Nat. Biotechnol. 37:424-429.

In some embodiments, the sequencing method comprises oxidative bisulfite sequencing (oxBS-Seq). In this method, potassium perruthenate can be used to convert 5-hydroxymethylcytosine into 5-formylcytosine without affecting 5-methylcytosine. Bisulfite treatment can then convert 5-formylcytosine to uracil.

In some embodiments, the sequencing method comprises APOBEC-coupled epigenetic sequencing (ACE-seq). In this method, Apolipoprotein B mRNA editing enzyme subunit 3A (APOBEC3A) is used to deaminate cytosine and 5-methylcytosine and sequence them as thymine, while β-glucosyltransferase (βGT) is used to convert 5-hydroxymethylcytosine into β-glucosyl hydroxymethylcytosine (which blocks deamination by APOBEC).

In some embodiments, the sequencing method comprises methylated DNA immunoprecipitation sequencing, such as methylated DNA immunoprecipitation (MeDIP) or hydroxymethylated DNA immunoprecipitation (hMeDIP) sequencing. In these techniques, antibodies specific to 5-methylcytosine or 5-hydroxymethylcytosine are used to isolate methylated DNA from total DNA by immunoprecipitation, following by purification and sequencing.

In some embodiments, the sequencing method comprises methylation array. In this method, microarray technology can be used to interrogate methylation status at multiple genomic loci. For example, DNA can be bisulfite treated, and oligonucleotide probes can be designed to detect unmethylated (by detecting uracil) or methylated (by detecting cytosine) versions of the same loci. Detection of which probe is hybridized to a sequence identifies whether the sequence was methylated or not.

In some embodiments, the sequencing method comprises reduced representation bisulfite sequencing (RRBS-Seq). In this method, DNA is digested with a methylation-insensitive restriction enzyme (e.g., Mspl), and sequence adaptors are added onto fragments after repair of sticky ends and A-tailing. DNA can then be treated with disulfide, amplified by PCR, and sequenced. See, e.g., Meissner, A. et al. (2005) Nucleic Acids Res. 33:5868-5877.

In some embodiments, the sequencing method comprises cytosine 5-hydroxymethylation sequencing, e.g., hMe-Seal. In this method, β-glucosyltransferase (βGT) is used to transfer a glucose moiety containing an azide group onto 5-hydroxymethylcytosine, which can be chemically modified with biotin to allow for detection, affinity enrichment, and sequencing of DNA fragments. See, e.g., Song, C. X. et al. (2011) Nat. Biotechnol. 29:68-72.

In some embodiments, the sequencing comprises whole genome sequencing (WGS). The sequencing may be performed at a depth sufficient to assess tumor progression or tumor non-progression in a subject with a desired performance (e.g., accuracy, sensitivity, specificity, positive predictive value (PPV), negative predictive value (NPV), or the area under curve (AUC) of a receiver operator characteristic (ROC)). In some embodiments, the sequencing is performed in a “low-pass” manner, for example, at a depth of no more than about 12×, no more than about 11×, no more than about 10×, no more than about 9×, no more than about 8×, no more than about 7×, no more than about 6×, no more than about 5×, no more than about 4×, no more than about 3.5×, no more than about 3×, no more than about 2.5×, no more than about 2×, no more than about 1.5×, or no more than about 1×.

In some embodiments, assessing tumor progression or tumor non-progression in a subject may comprise aligning the cfDNA sequencing reads to a reference genome. The reference genome may comprise at least a portion of a genome (e.g., the human genome). The reference genome may comprise an entire genome (e.g., the entire human genome). The reference genome may comprise an entire genome with certain base conversions applied (e.g., the entire human genome with non-methylated cytosines converted to thymines), as may be used for methylation data alignment. The reference genome may comprise a database comprising a plurality of genomic regions that correspond to coding and/or non-coding genomic regions of a genome. The database may comprise a plurality of genomic regions that correspond to cancer-associated (or tumor-associated) coding and/or non-coding genomic regions of a genome, such as cancer driver mutations (e.g., single nucleotide variants (SNVs), copy number alterations (CNAs), insertions or deletions (indels) and other rearrangements, fusion genes, and genomic regions (such as mononucleotides and/or dinucleotides)). The alignment may be performed using a Burrows-Wheeler algorithm (BWA), a sambamba algorithm, a samtools algorithm, or any other suitable alignment algorithm.

In some embodiments, assessing tumor progression or tumor non-progression in a subject may comprise generating a quantitative measure of the cfDNA sequencing reads for each of a plurality of genomic regions. Quantitative measures of the cfDNA sequencing reads may be generated, such as counts of DNA sequencing reads that are aligned with a given genomic region. CfDNA sequencing reads having a portion or all of the sequencing read aligning with a given genomic region may be counted toward the quantitative measure for that genomic region.

In some embodiments, genomic regions may comprise tumor markers. Patterns of specific and non-specific genomic regions may be indicative of tumor progression or tumor non-progression status. Changes over time in these patterns of genomic regions may be indicative of changes in tumor progression or tumor non-progression status.

In some embodiments, cfDNA may be assayed by performing binding measurements of the plurality of cfDNA molecules at each of the plurality of genomic regions. In some embodiments, performing the binding measurements comprises assaying the plurality of cfDNA molecules using probes that are selective for at least a portion of a plurality of genomic regions in the plurality of cfDNA molecules. In some embodiments, the probes are nucleic acid molecules having sequence complementarity with nucleic acid sequences of the plurality of genomic regions. In some embodiments, the nucleic acid molecules are primers or enrichment sequences. In some embodiments, the assaying comprises use of array hybridization or polymerase chain reaction (PCR), or nucleic acid sequencing.

In some embodiments, the method further comprises enriching the plurality of cfDNA molecules for at least a portion of the plurality of genomic regions. In some embodiments, the enrichment comprises amplifying the plurality of cfDNA molecules. For example, the plurality of cfDNA molecules may be amplified by selective amplification (e.g., by using a set of primers or probes comprising nucleic acid molecules having sequence complementarity with nucleic acid sequences of the plurality of genomic regions). Alternatively or in combination, the plurality of cfDNA molecules may be amplified by universal amplification (e.g., by using universal primers). In some embodiments, the enrichment comprises selectively isolating at least a portion of the plurality of cfDNA molecules (e.g., a portion of the plurality of cfDNA molecules which are enriched for shorter cfDNA molecules).

In some embodiments, the methods of the present disclosure comprise obtaining one or more quantitative measure(s), e.g., of fragment length, number of nucleotides, and so forth. In some embodiments, the quantitative measure(s) are statistical measure(s). Suitable statistical measures are known in the art. For example, in some embodiments, the statistical measure of deviation comprises a z-score relative to a set of reference samples or a set of reference values (e.g., a set of baseline values).

In some embodiments, the method of assessing tumor progression or tumor-non-progression in a subject comprises processing the plurality of counts to obtain a quantitative measure (e.g., a statistical measure) of fragment length of the plurality of cfDNA molecules. In some embodiments, the quantitative measures of fragment length of the plurality of cfDNA molecules comprise a number of nucleotides of each of the plurality of cfDNA molecules. The reference samples may be obtained from one or more subjects having a tumor progression and/or from subjects not having a tumor progression (e.g., subjects having a tumor non-progression or unaffected patients). The reference samples may be obtained from one or more subjects having a cancer type or from subjects not having a cancer type (e.g., brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, urinary tract cancer). The reference samples may be obtained from one or more subjects having advanced-stage cancer or not having advanced-stage cancer (e.g., an earlier-stage cancer or no cancer).

In some embodiments, the cfDNA sequencing reads may be normalized or corrected. For example, the cfDNA sequencing reads may be de-deduplicated, normalized, and/or corrected to account for known biases in sequencing and library preparation and/or known biases in sequencing and library preparation. In some embodiments, a subset of the quantitative measures (e.g., statistical measures) may be filtered out, e.g., based on whether the changes in such quantitative measures (e.g., across different time points) are significantly different from those observed in unaffected subjects (e.g., a background profile of cfDNA molecules). For example, quantitative measures may be filtered out when an absolute value of the z-score of the quantitative measure is less than (or no more than) a pre-determined number. The pre-determined number may be about 0.1, about 0.2, about 0.5, about 1, about 1.5, about 2, about 2.5, about 3, about 3.5, about 4, about 4.5, about 5, or more than about 5.

In some embodiments, the plurality of genomic regions comprises mononucleotides and/or dinucleotides. The plurality of genomic regions may comprise at least about 10 distinct genomic regions, at least about 50 distinct genomic regions, at least about 100 distinct genomic regions, at least about 500 distinct genomic regions, at least about 1 thousand distinct genomic regions, at least about 5 thousand distinct genomic regions, at least about 10 thousand distinct genomic regions, at least about 50 thousand distinct genomic regions, at least about 100 thousand distinct genomic regions, at least about 500 thousand distinct genomic regions, at least about 1 million distinct genomic regions, at least about 2 million distinct genomic regions, at least about 3 million distinct genomic regions, at least about 4 million distinct genomic regions, at least about 5 million distinct genomic regions, at least about 10 million distinct genomic regions, at least about 15 million distinct genomic regions, at least about 20 million distinct genomic regions, at least about 25 million distinct genomic regions, at least about 30 million distinct genomic regions, or more than 30 million distinct genomic regions.

In some embodiments, the region(s) of the genome comprises one or more MAGE (melanoma-associated antigen) genes, e.g., human MAGE genes. In some embodiments, the region of the genome comprises one or more promoters corresponding to one or more MAGE (melanoma-associated antigen) genes, e.g., human MAGE genes. MAGE genes (e.g., human MAGE genes) are known in the art; see, e.g., Chomez, P. et al. (2001) Cancer Res. 61:5544-5551 and Weon, J. L. and Potts, P. R. (2015) Curr. Opin. Cell Biol. 37:1-8. Exemplary MAGE genes (e.g., human MAGE genes) include, without limitation, MAGE-A genes (e.g., MAGE-AL MAGE-A2, MAGE-A2B, MAGE-A3, MAGE-A4, MAGE-A5, MAGE-A6, MAGE-A8, MAGE-A9, MAGE-A10, MAGE-A11, and MAGE-A12), MAGE-B genes (e.g., MAGE-B1, MAGE-B2, MAGE-B3, MAGE-B4, MAGE-B5, MAGE-B6, MAGE-B6B, MAGE-B10, MAGE-B16, MAGE-B17, and MAGE-B18), MAGE-C genes (e.g., MAGE-C1, MAGE-C2, and MAGE-C3), and Type II MAGE genes (e.g., MAGE-D1, MAGE-D2, MAGE-D3, MAGE-D4, MAGE-E1, MAGE-E2, MAGE-F1, MAGE-G1, MAGE-H1, MAGE-L2, NDN, and NDNL2).

In some embodiments, the tumor progression of the subject is detected with a sensitivity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor progression of the subject is detected with a specificity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor progression of the subject is detected with a positive predictive value (PPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor progression of the subject is detected with a negative predictive value (NPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor progression of the subject is detected with an area under curve (AUC) of a receiver operator characteristic (ROC) of at least about 0.5, at least about 0.6, at least about 0.7, at least about 0.75, at least about 0.8, at least about 0.85, at least about 0.9, at least about 0.95, at least about 0.96, at least about 0.97, at least about 0.98, or at least about 0.99.

In some embodiments, the method of assessing tumor progression in a subject further comprises determining a tumor non-progression of the subject when the tumor progression is not detected.

In some embodiments, the tumor non-progression of the subject is detected with a sensitivity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor non-progression of the subject is detected with a specificity of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor non-progression of the subject is detected with a positive predictive value (PPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor non-progression of the subject is detected with a negative predictive value (NPV) of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

In some embodiments, the tumor non-progression of the subject is detected with an area under curve (AUC) of a receiver operator characteristic (ROC) of at least about 0.5, at least about 0.6, at least about 0.7, at least about 0.75, at least about 0.8, at least about 0.85, at least about 0.9, at least about 0.95, at least about 0.96, at least about 0.97, at least about 0.98, or at least about 0.99.

In some embodiments, the subject has been diagnosed with cancer. For example, the cancer may be one or more types, including, without limitation: brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.

In some embodiments, the method further comprises, based on the determined tumor progression of the subject, administering a therapeutically effective amount of a treatment to treat the tumor of the subject. In some embodiments, the treatment comprises treatment with surgery, chemotherapy, a therapeutic agent, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor.

The tumor progression or tumor non-progression of a subject may be assessed to determine a diagnosis of a cancer, prognosis of a cancer, recurrence of a cancer, or an indication of progression or regression of a tumor in the subject. In addition, one or more clinical outcomes may be assigned based on the tumor progression or tumor non-progression assessment or monitoring (e.g., a difference in tumor progression or tumor non-progression status between two or more time points). Such clinical outcomes may include diagnosing the subject with a cancer comprising tumors of one or more types, diagnosing the subject with the cancer comprising tumors of one or more types and stages, prognosing the subject with the cancer (e.g., indicating a clinical course of treatment (e.g., surgery, chemotherapy, a therapeutic agent, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor, or other treatment) for the subject, indicating another clinical course of action (e.g., no treatment, continued monitoring such as on a prescribed time interval basis, stopping a current treatment, switching to another treatment), or indicating an expected survival time for the subject.

In some embodiments, the method of assessing tumor progression of a subject further comprises processing the first plurality of CNAs with the second plurality of CNAs to determine a CNA profile change. In some embodiments, the method of assessing tumor progression of a subject further comprises processing the first plurality of fragment lengths with the second plurality of fragment lengths to determine a fragment length profile change. In some embodiments, the method of assessing tumor progression of a subject further comprises determining a first tumor fraction of the subject at the first timepoint or a second tumor fraction of the subject at the second timepoint, based at least in part on the CNA profile change and the fragment length profile change. In some embodiments, the method of assessing tumor progression of a subject further comprises detecting a tumor progression of the subject based at least in part on the first tumor fraction or the second tumor fraction. For example, the tumor progression may be determined based on whether the first tumor fraction or the second tumor fraction meets a pre-determined criterion (e.g., being at least a pre-determined threshold, being greater than a pre-determined threshold, being at most a pre-determined threshold, or being less than a pre-determined threshold). The pre-determined threshold may be generated by performing the tumor progression or tumor non-progression assessment on one or more reference samples obtained or derived from one or more reference subjects (e.g., patients known to have a certain tumor type, patients known to have a certain tumor type of a certain stage, or healthy subjects not exhibiting any cancer) and identifying a suitable pre-determined threshold based on the tumor progression or tumor non-progression of the reference samples obtained or derived from the reference subjects.

The pre-determined threshold may be adjusted based on a desired sensitivity, specificity, positive predictive value (PPV), negative predictive value (NPV), or accuracy of assessing the tumor progression or tumor non-progression status of a subject. For example, the pre-determined threshold may be adjusted to be lower if a high sensitivity of assessing the tumor progression or tumor non-progression status of a subject is desired. Alternatively, the pre-determined threshold may be adjusted to be higher if a high specificity assessing the tumor progression or tumor non-progression status of a subject is desired. The pre-determined threshold may be adjusted so as to achieve a desired balance between false positives (FPs) and false negatives (FNs) in assessing obtained or derived from one or more reference subjects of a cancer comprising a tumor of one or more types.

In some embodiments, the method of assessing tumor progression or tumor non-progression further comprises repeating the assessment at a second later time point. The second time point may be chosen for a suitable comparison of tumor progression or tumor non-progression assessment relative to the first time point. Examples of second time points may correspond to a time after surgical resection, a time during treatment administration or after treatment administration to treat the cancer in the subject to monitor efficacy of the treatment, or a time after cancer is undetectable in the subject after treatment to monitor for residual disease or cancer recurrence in the subject.

In some embodiments, the methods of the present disclosure include determining a difference in status (e.g., tumor progression or tumor non-progression status) at two or more distinct time points. The difference in status between time points (e.g., comparing status at an earlier point versus status at a later or current point) can be used, e.g., to indicate progression, regression, recurrence, or stable status of a tumor. In some embodiments, differences in status over time can be plotted, e.g., in order to represent progression, regression, recurrence, or stable status of a tumor. For example, in some embodiments, a method of assessing tumor progression or tumor non-progression further comprises determining a difference between a first tumor progression/tumor non-progression status and a second tumor progression/tumor non-progression status. In some embodiments, the difference is indicative of a progression or regression of a tumor of the subject. Alternatively or in combination, the method may further comprise generating, by a computer processor, a plot of a first tumor progression/tumor non-progression status and a second tumor progression/tumor non-progression status as a function of a first and a second time point. In some embodiments, the plot is indicative of the progression or regression of the tumor of the subject. For example, the computer processor may generate a plot of the two or more tumor progression/tumor non-progression statuses on a y-axis against the times corresponding to the time of collection for the data corresponding to the two or more tumor progression or tumor non-progression statuses on an x-axis.

A difference in tumor status over time, such a difference determined or plotted as described supra between a first tumor progression/non-progression status and a second tumor progression/non-progression status, may be indicative of progression, regression, recurrence, or stable status of a tumor in a subject. For example, if a later tumor progression/non-progression status (e.g., a second status) is larger than an earlier tumor progression/non-progression status (e.g., a first status), this difference may indicate, e.g., tumor progression, inefficacy of a treatment to the tumor in the subject, resistance of the tumor to an ongoing treatment, metastasis of the tumor to other sites in the subject, or residual disease or cancer recurrence in the subject. If a later tumor progression/non-progression status (e.g., a second status) is smaller than an earlier tumor progression/non-progression status (e.g., a first status), this difference may indicate, e.g., tumor regression, efficacy of a surgical resection of the tumor in the subject, efficacy of a treatment to the tumor in the subject, or lack of residual disease or cancer recurrence in the subject.

After assessing and/or monitoring tumor progression or tumor non-progression status, one or more clinical outcomes may be assigned based on the tumor progression or tumor non-progression status assessment or monitoring (e.g., a difference in tumor progression or tumor non-progression status between two or more time points). Such clinical outcomes may include diagnosing the subject with a cancer comprising tumors of one or more types, diagnosing the subject with the cancer comprising tumors of one or more types and stages, prognosing the subject with the cancer (e.g., indicating a clinical course of treatment (e.g., surgery, chemotherapy, a therapeutic agent, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, a checkpoint inhibitor, or other treatment) for the subject, or identifying the origin of the tumor cDNA within the subject, indicating another clinical course of action (e.g., no treatment, continued monitoring such as on a prescribed time interval basis, stopping a current treatment, switching to another treatment), or indicating an expected survival time for the subject.

### Treatments

Certain aspects of the present disclosure relate to treatments, e.g., with one or more therapeutic agent(s). For example, in some embodiments, the treatment can include treatment with surgery, chemotherapy, a therapeutic agent, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor. Exemplary and non-limiting descriptions of treatments are provided herein.

For example, the treatment can be with a cytotoxic or a cytostatic agent. Exemplary cytotoxic agents include anti-microtubule agents, topoisomerase inhibitors, taxanes, antimetabolites, mitotic inhibitors, alkylating agents, intercalating agents, agents capable of interfering with a signal transduction pathway, and agents that promote apoptosis and radiation. In yet other embodiments, the methods can be used in combination with immunodulatory agents, e.g., IL-1, 2, 4, 6, or 12, or interferon alpha or gamma, or immune cell growth factors such as GM-CSF.

In some embodiments, the treatment can be an immunotherapeutic or immunomodulating therapy, e.g., a compound-, antibody-, or cell-based immunotherapy. Examples of immunotherapies include, without limitation, a checkpoint inhibitor, cancer vaccine, cell-based therapy, T cell receptor (TCR)-based therapy, adjuvant immunotherapy, cytokine immunotherapy, or oncolytic virus therapy. In some embodiments, the cancer immunotherapy comprises a small molecule, nucleic acid, polypeptide, carbohydrate, toxin, cell-based, or binding agent therapeutic agent. Examples of cancer immunotherapies are described in greater detail infra but are not intended to be limiting.

In some embodiments, the cancer immunotherapy comprises one or more of: a checkpoint inhibitor, cancer vaccine, cell-based therapy, T cell receptor (TCR)-based therapy, adjuvant immunotherapy, cytokine immunotherapy, and oncolytic virus therapy. In some embodiments, the cancer immunotherapy comprises small molecule, nucleic acid, polypeptide, carbohydrate, toxin, cell-based, or binding agent therapeutic agent. Examples of cancer immunotherapies are described in greater detail infra but are not intended to be limiting. In some embodiments, the cancer immunotherapy activates one or more aspects of the immune system to attack a cell (e.g., a tumor cell) that expresses a neoantigen of the present disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against the tumor (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPS) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-tumor response.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TGO2 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into the patient (possibly with proinflammatory or other attractants such as GM-CSF), taken up by cells in vivo to make the specific antigens, which would then provoke the desired immune response. In some embodiments, vectors may be used to deliver more than one tumor antigen at a time, to increase the immune response. In addition, recombinant virus, bacteria or yeast vectors should trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA, that encodes an antigenic protein, to elicit a protective immune response has been demonstrated in numerous experimental systems. Vaccination through directly injecting DNA, that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide (i.e. comprising HPV antigens), or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) tumor cells. In some embodiments, the immune response results in inhibition of tumor and/or metastatic cell growth and/or proliferation and in related embodiments results in neoplastic cell death and/or resorption. The immune cells can be derived from a different organism/host (exogenous immune cells) or can be cells obtained from the subject organism (autologous immune cells). In some embodiments the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, the population of immune cells can be obtained from a subject in need of therapy or suffering from a disease associated with reduced immune cell activity. Thus, the cells will be autologous to the subject in need of therapy. In some embodiments, the population of immune cells can be obtained from a donor, such as a histocompatibility matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. In some embodiments, the T cells are derived from the blood, bone marrow, lymph, umbilical cord, or lymphoid organs. In some aspects, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation. In some embodiments, the sub-types and subpopulations of T cells (e.g., CD4⁺ and/or CD8⁺ T cells) are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, one or more of the T cell populations is enriched for or depleted of cells that are positive for a specific marker, such as surface markers, or that are negative for a specific marker. In some embodiments, such markers are those that are absent or expressed at relatively low levels on certain populations of T cells (e.g., non-memory cells) but are present or expressed at relatively higher levels on certain other populations of T cells (e.g., memory cells). In some embodiments, T cells are separated from a PBMC sample by negative selection of markers expressed on non-T cells, such as B cells, monocytes, or other white blood cells, such as CD 14. In some embodiments, a CD4+ or CD8⁺ selection step is used to separate CD4+ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. In some embodiments, CD8⁺ T cells are further enriched for or depleted of naive, central memory, effector memory, and/or central memory stem cells, such as by positive or negative selection based on surface antigens associated with the respective subpopulation. In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, such as from about 10 to about 14 days.

In some embodiments, the cultured T cells can be pooled and rapidly expanded. Rapid expansion provides an increase in the number of antigen-specific T-cells, e.g., of at least about 50-fold (e.g., 50-, 60-, 70-, 80-, 90-, or 100-fold, or greater) over a period of about 10 to about 14 days. In some embodiments, expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being particularly contemplated. The non-specific T-cell receptor stimulus can include around 30 ng/ml of OKT3, a mouse monoclonal anti-CD3 antibody (available from Ortho-McNeil®, Raritan, N.J.). In some embodiments, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2 or IL-15, with IL-2 being contemplated. The in vv/ro-induced T-cells are rapidly expanded by re stimulation with the same antigen(s) of the cancer pulsed onto HLA-A2-expressing antigen-presenting cells. In some embodiments, the T cells can be re-stimulated with irradiated, autologous lymphocytes or with irradiated HLA-A2+ allogeneic lymphocytes and IL-2, for example. In some embodiments, the autologous T-cells can be modified to express a T-cell growth factor that promotes the growth and activation of the autologous T-cells. In some embodiments, suitable T-cell growth factors include, for example, interleukin (IL)-2, IL-7, IL-15, and IL-12. Suitable methods of modification are known in the art. See, for instance, Sambrook et al, MOLECULAR CLONING: A LABORATORY MANUAL, 3^{rd} ed., Cold Spring Harbor Press, Cold Spring Harbor, N.Y. 2001; and Ausubel et al, CURRENT PROTOCOLS IN MOLECULAR BIOLOGY, Greene Publishing Associates and John Wiley & Sons, NY, 1994. In some embodiments, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression. In some embodiments, autologous T cells may be engineered to express a defined T cell receptor (TCR) that are directed against target TAAs, either wild-type TCR, or mutated/engineered TCR towards a higher affinity to the antigen peptide/MHC molecule complexes. In some embodiments, autologous T cells may be engineered to express a CAR, e.g., as described infra.

In some embodiments, the T cell-based therapy comprises a chimeric antigen receptor (CAR)-T-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen of the present disclosure.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen of the present disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize a neoantigen of the present disclosure. In some embodiments, the TILs are exposed to one or more neoantigens of the present disclosure in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of interleukin 2 (IL-2), strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD 16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. In some embodiments, umbilical CB is used to derive NK cells. In some embodiments, the NK cells are isolated and expanded by the previously described method of ex vivo expansion of NK cells (Spanholtz et al, 2011; Shah et al, 2013). In some embodiments, CB mononuclear cells are isolated by ficoll density gradient centrifugation and cultured in a bioreactor with IL-2 and artificial antigen presenting cells (aAPCs). After 7 days, the cell culture is depleted of any cells expressing CD3 and re-cultured for an additional 7 days. The cells are again CD3-depleted and characterized to determine the percentage of CD56⁺/CD3 cells or NK cells. In some embodiments, umbilical CB is used to derive NK cells by the isolation of CD34⁺ cells and differentiation into CD56⁺/CD3 cells by culturing in medium contain SCF, IL-7, IL-15, and IL-2.

In some embodiments, the cell-based therapy comprises a dendritic cell-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens, e.g., a neoantigen of the present disclosure, to the patient's immune system. In some embodiments, the vaccine comprises dendritic cells that have been exposed to one or more neoantigens of the present disclosure. In some embodiments, the vaccine comprises dendritic cells that present one or more neoantigens of the present disclosure, e.g., via MEC class I. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based biologics that specifically bind a neoantigen of the present disclosure. For example, the TCR-based therapeutic may comprise a TCR or extracellular portion thereof that specifically binds a neoantigen of the present disclosure (e.g., as presented on a cell surface via MEC class I) as well as a moiety that binds an immune cell (e.g., a T cell), such as an antibody or antibody fragment that specifically binds a T cell surface protein or receptor (e.g., an anti-CD3 antibody or antibody fragment).

In some embodiments, the cancer immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the cancer immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the cancer immunotherapy comprises oncolytic virus therapy. Oncolytic virus therapy uses genetically modified viruses to replicate in and kill cancer cells, leading to the release of antigens (e.g., a neoantigen of the present disclosure) that stimulate an immune response. In some embodiments, replication-competent oncolytic viruses expressing a tumor antigen comprise any naturally occurring (e.g. from a “field source”) or modified replication-competent oncolytic virus. In some embodiments, the oncolytic virus, in addition to expressing a tumor antigen, may be modified to increase selectivity of the virus for cancer cell. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, the replicative oncolytic vaccinia virus expressing a tumor antigen may be engineered to lack one or more functional genes in order to increase the cancer selectivity of the virus. In some embodiments, the oncolytic vaccinia virus is engineered to lack thymidine kinase (TK) activity. In some embodiments, the oncolytic vaccinia virus may be engineered to lack vaccinia virus growth factor (VGF). In some embodiments, the oncolytic vaccinia virus may be engineered to lack both VFG and TK activity. In some embodiments, the oncolytic vaccinia virus may be engineered to lack one or more genes involved in evading host interferon (IFN) response such as E3L, K3L, B18R, or B8R. In some embodiments, the replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen of the combination may be locally or systemically administered to a subject, e.g. via intratumoral, intraperitoneal, intravenous, intra-arterial, intramuscular, intradermal, intracranial, subcutaneous, or intranasal administration.

In some embodiments, the cancer immunotherapy comprises a checkpoint inhibitor. As is known in the art, a checkpoint inhibitor targets at least one immune checkpoint protein to alter the regulation of an immune response, eg., down-modulating or inhibiting an immune response. Immune checkpoint proteins include, e.g., CTLA4, PD-L1, PD-1, PD-L2, VISTA, B7-H2, B7-H3, B7-H4, B7-H6, 2B4, ICOS, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1 , TIM-3, TIM-4, LAG-3, BTLA, SIRPalpha (CD47), CD48, 2B4 (CD244), B7,1 B7.2, ILT-2, ILT-4 TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GALS, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-IBB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, a checkpoint inhibitor decreases the activity of a checkpoint protein that negatively regulates immune cell function, e.g., in order to enhance T cell activation and/or an anti-cancer immune response; in other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. In some embodiments, the checkpoint inhibitor is an antibody. Examples of checkpoint inhibitors include, without limitation, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). In some embodiments, known inhibitors of the immune checkpoint proteins or analogs thereof may be used, in particular chimerized, humanized or human forms of antibodies may be used.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2,” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD2 73,” “B7-DC,” “Btd.c,” and “PDL2.” An exemplary human PD-L2 is shown in UniProtKB/Swiss-Prot Accession No. Q9BQ51. In some instances, PD-1 PD-L1, and PD-L2 are human PD-1 , PD-L1 and PD-L2.

In some instances, the PD-1 binding antagonist is a molecule that inhibits the binding of PD-1 to its ligand binding partners. in a specific aspect the PD-1 ligand binding partners are PD-L1 and/or PD-L2. In another instance, a PD-L1 binding antagonist is a molecule that inhibits the binding of PD-L1 to its binding ligands. In a specific aspect, PD-L1 binding partners are PD-1 and/or B7-1. In another instance, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide. In some embodiments, the PD-1 binding antagonist is a small molecule, a nucleic acid, a polypeptide (e.g., antibody), carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is selected from the group consisting of MDX-1 106 (nivolumab), MK-3475 (pembrolizumab), _MEDT-0680 (AMP-514), PDR001, REGN2810, MGA-012, JNJ-63723283, BI 754091, and BGB-108. MDX-1 106, also known as MDX-1 106-04, ONO-4538, BMS-936558, or nivolumab, is an anti-PD-1 antibody described in WO2006/121 168. MK-3475, also known as pembrolizumab or lambrolizumab, is an anti-PD-1 antibody described in WO 2009/1 14335. In some instances, the PD-1 binding antagonist is an immunoadhesin (e.g., an immunoadhesin comprising an extracellular or PD-1 binding portion of PD-L1 or PD-L2 fused to a. constant region (e.g., an Fc region of an immunoglobulin sequence). In some instances, the PD-1 binding antagonist is AMP-224. AMP-224, also known as B7-DCIg, is a PD-L2-Fc fusion soluble receptor described in WO 2010/027827 and WO 2011 /066342.

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168. In some embodiments, the anti-PD-1 antibody comprises a heavy chain and a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 1)
QVQLVESGGGVVQPGRSLRLDCKASGITFSNSGMHWVRQAPGKGLEWVA
VIWYDGSKRYYADSVKGRFTISRDNSKNTLFLQMNSLRAEDTAVYYCAT
NDDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLG,

and

(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)
EIVLTQSPATLSLSPGERATLSCRASQSVSSYLAWYQQKPGQAPRLLIY
DASNRATGIPARFSGSGSGTDFTLTISSLEPEDFAVYYCQQSSNWPRTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the anti-PD-1 antibody comprises the six HVR sequences from SEQ ID NO:1 and SEQ ID NO:2 (e.g., the three heavy chain HVRs from SEQ ID NO:1 and the three light chain HVRs from SEQ ID NO:2). In some embodiments, the anti-PD-1 antibody comprises the heavy chain variable domain from SEQ ID NO:1 and the light chain variable domain from SEQ ID NO:2.

In some embodiments, the anti-PD-1 antibody is pembrolizumab (CAS Registry Number: 1374853-91-4). Pembrolizumab (Merck), also known as MK-3475, Merck 3475, lambrolizumab, KEYTRUDA®, and SCH-900475, is an anti-PD-1 antibody described in WO2009/114335. In some embodiments, the anti-PD-1 antibody comprises a heavy chain and a light chain sequence, wherein:
(a) the heavy chain sequence has at least 85%, at least 90%, at least 91%, at least 92%, at least 93%, at least 94©, at least 95%, at least 96%, at least 97%, at least 98%, at least 99% or 100% sequence identity to the heavy chain sequence:

(SEQ ID NO: 3)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLG,

and
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC .

In some embodiments, the anti-PD-1 antibody comprises the six HVR sequences from SEQ ID NO:3 and SEQ ID NO:4 (e.g., the three heavy chain HVRs from SEQ ID NO:3 and the three light chain HVRs from SEQ ID NO:4). In some embodiments, the anti-PD-1 antibody comprises the heavy chain variable domain from SEQ ID NO:3 and the light chain variable domain from SEQ ID NO:4.

Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZMO09, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, CCX-4503, or a derivative thereof. In some embodiments, the PD-1 binding antagonist is a peptide or small molecule compound. In some embodiments, the PD-1 binding antagonist is AUNP-12 (PierreFabre/Aurigene). In some embodiments, the PD-1 axis binding antagonist comprises a small molecule PD-1 axis binding antagonist described in Guzik et al., Molecules (2019) May 30;24(11). Other PD-1 inhibitors for use in the methods provided herein are known in the art such as described in U.S. Pat. Nos. 8,735,553, 8,354,509, and 8,008,449.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In any of the instances herein, the isolated anti-PD-L1 antibody can bind to a human PD-L1 for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting binding between PD-L1 and PD-1 and/or between PD-L1 and B7-1 , In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody, In some instances, the anti-PD-L1 antibody is a. human antibody. In some instances, the anti-PD-L1 antibody is selected from the group consisting of YW243.55.S70, MPDL3280A (atezolizumab), MDX-1 105, and MEDI4736 (durvalumab), and MSB0010718C (avelumab). Antibody YW243.55.S70 is an anti-PD-L1 described in WO 2010/077634, MDX-1 105, also known as BMS-936559, is an anti-PD-L1 antibody described in WO2007/005874. MEDI4736 (durvaluma.b) is an anti-PD-L1 monoclonal antibody described in WO201 1 /066389 and US2013/034559. Examples of anti-PD-L1 antibodies useful for the methods of this disclosure, and methods for making thereof are described in PCT patent application WO 2010/077634, W( )2007/005874, WO 2011/066389, U.S. Pat. No. 8,217,149, and US2013/034559. In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, FS-118, or a derivative thereof.

In some embodiments, the anti-PDL1 antibody comprises a heavy chain variable region and a light chain variable region, wherein:
(a) the heavy chain variable region comprises an HVR-H1, HVR-H2, and HVR-H3 sequence of GFTFSDSWIH (SEQ ID NO:5), AWISPYGGSTYYADSVKG (SEQ ID NO:6) and RHWPGGFDY (SEQ ID NO:7), respectively, and
(b) the light chain variable region comprises an HVR-L1, HVR-L2, and HVR-L3 sequence of RASQDVSTAVA (SEQ ID NO:8), SASFLYS (SEQ ID NO:9) and QQYLYHPAT (SEQ ID NO:10), respectively.

In some embodiments, the anti-PDL1 antibody is MPDL3280A, also known as atezolizumab and TECENTRIQ® (CAS Registry Number: 1422185-06-5). In some embodiments, the anti-PDL1 antibody comprises a heavy chain and a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 11)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,

and
(b) the light chain sequence has at least 85%, at least 90%, at least 91 at least 92%, at least 93%, at least 94%, at least 95%, at least 96%, at least 97%, at least 98%, at least 99% or 100% sequence identity to the light chain sequence:

(SEQ ID NO: 12)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some embodiments, the anti-PDL1 antibody comprises a heavy chain and a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 13)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,

and
(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 14)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC .

In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:14. In some instances, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 13. In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:14 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO:13.

In a still further specific aspect, the antibody further comprises a human or murine constant region. In a still further aspect, the human constant region is selected from the group consisting of IgG1, gG2, IgG2, IgG3, and IgG4. In a still further specific aspect, the human constant region is IgG1. In a still further aspect, the murine constant region is selected from the group consisting of IgG1,IgG2A, IgG2B, and IgG3. In a still further aspect, the murine constant region in IgG2A. In a still further specific aspect, the antibody has reduced or minimal effector function. In a still further specific aspect the minimal effector function results from an “effector-less Fc mutation” or aglycosylation. In still a further instance, the effector-less Fc mutation is an N297A or D265A/N297A substitution in the constant region.

In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxy lysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In some embodiments, the anti-PDL1 antibody is avelumab (CAS Registry Number: 1537032-82-8). Avelumab, also known as MSB0010718C, is a human monoclonal IgG1 anti-PDL1 antibody (Merck KGaA, Pfizer). In some embodiments, the anti-PDL1 antibody comprises a heavy chain and a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 15)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG,

and

(b) the light chain sequence has at least 85%, at least 90%, at least 91%, at least 92%, at least 93%, at least 94%, at least 95%, at least 96%. at least 97%, at least 98%, at least 99% or 100% sequence identity to the light chain sequence:

(SEQ ID NO: 16)
QSALTQPASVSGSPGQSITISCTGTSSDVGGYNYVSWYQQHPGKAPKLM
IYDVSNRPSGVSNRFSGSKSGNTASLTISGLQAEDEADYYCSSYTSSST
RVFGTGTKVTVLGQPKANPTVTLFPPSSEELQANKATLVCLISDFYPGA
VTVAWKADGSPVKAGVETTKPSKQSNNKYAASSYLSLTPEQWKSHRSYS
CQVTHEGSTVEKTVAPTECS.

In some embodiments, the anti-PDL1 antibody comprises the six HVR sequences from SEQ ID NO:15 and SEQ ID NO:16 (e.g., the three heavy chain HVRs from SEQ ID NO:15 and the three light chain HVRs from SEQ ID NO:16). In some embodiments, the anti-PDL1 antibody comprises the heavy chain variable domain from SEQ ID NO:15 and the light chain variable domain from SEQ ID NO:16.

In some embodiments, the anti-PDL1 antibody is durvalumab (CAS Registry Number: 1428935-60-7). Durvalumab, also known as MEDI4736, is an Fc optimized human monoclonal IgG1 kappa anti-PDL1 antibody (MedImmune, AstraZeneca) described in WO2011/066389 and US2013/034559. In some embodiments, the anti-PDL1 antibody comprises a heavy chain and a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 17)
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPEFEGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPASIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPG,

and
(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence

(SEQ ID NO: 18)
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In some embodiments, the anti-PDL1 antibody comprises the six HVR sequences from SEQ ID NO:17 and SEQ ID NO:18 (e.g., the three heavy chain HVRs from SEQ ID NO:17 and the three light chain HVRs from SEQ ID NO:18). In some embodiments, the anti-PDL1 antibody comprises the heavy chain variable domain from SEQ ID NO:17 and the light chain variable domain from SEQ ID NO:18.

Other examples of anti-PD-L1 antibodies include, but are not limited to, MDX-1105 (BMS-936559; Bristol Myers Squibb), LY3300054 (Eli Lilly), STI-A1014 (Sorrento), KN035 (Suzhou Alphamab), FAZ053 (Novartis), or CX-072 (CytomX Therapeutics).

In some embodiments, the PD-L1 axis binding antagonist comprises small molecule PD-L1 axis binding antagonist GS-4224. In some embodiments, the PD-L1 axis binding antagonist comprises a small molecule PD-L1 axis binding antagonist described in PCT/US2019/017721.

In some embodiments, the checkpoint inhibitor is CT-011, also known as hBAT, hB AT-1 or pidilizumab, an antibody described in WO 2009/101611.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), carbohydrate, a lipid, a metal, or a toxin.

In some embodiments, the anti-CTLA4 antibody is ipilimumab (YERVOY®; CAS Registry Number: 477202-00-9). Ipilimumab, also known as BMS-734016, MDX-010, and MDX-101, is a fully human monoclonal IgG1 kappa anti-CTLA4 antibody (Bristol-Myers Squibb) described in WO2001/14424. In some embodiments, the anti-CTLA4 antibody comprises a heavy chain and a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 19)
QVQLVESGGGVVQPGRSLRLSCAASGFTFSSYTMHWVRQAPGKGLEWVT
FISYDGNNKYYADSVKGRFTISRDNSKNTLYLQMNSLRAEDTAIYYCAR
TGWLGPFDYWGQGTLVTVSS,

and
(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 20)
EIVLTQSPGTLSLSPGERATLSCRASQSVGSSYLAWYQQKPGQAPRLLI
YGAFSRATGIPDRFSGSGSGTDFTLTISRLEPEDFAVYYCQQYGSSPWT
FGQGTKVEIK.

In some embodiments, the anti-CTLA4 antibody comprises the six HVR sequences from SEQ ID NO:19 and SEQ ID NO:20 (e.g., the three heavy chain HVRs from SEQ ID NO:19 and the three light chain HVRs from SEQ ID NO:20). In some embodiments, the anti-CTLA4 antibody comprises the heavy chain variable domain from SEQ ID NO:19 and the light chain variable domain from SEQ ID NO:20.

Other examples of anti-CTLA4 antibodies include, but are not limited to, APL-509, AGEN1884, and CS1002. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, and CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceeding.

In some embodiments, the immune checkpoint inhibitor is monovalent and/or monospecific. In some embodiments, the immune checkpoint inhibitor is multivalent and/or multispecific.

In some embodiments, the immunotherapy comprises an immunoregulatory molecule or cytokine. An immunoregulatory profile is required to trigger an efficient immune response and balance the immunity in a subject. In some embodiments, the immunoregulatory molecule is in included with any of the treatments detailed herein. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present invention. Examples of chemokines include, but are not limited to, MIP-3a (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, and BCA-1 (Blc), as well as functional fragments thereof.

The compositions utilized in the methods described herein (e.g., cancer immunotherapies) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctival, intravesicularly, mucosally, intrapericardially, intraumbilically, intraocularly, intraorbitally, topically, transdermal, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, the checkpoint inhibitor is administered intravenously, intramuscularly, subcutaneously, topically, orally, transdermal, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Cancer immunotherapies (e.g., an antibody, binding polypeptide, and/or small molecule) described herein (any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent need not be, but is optionally formulated with and/or administered concurrently with one or more agents currently used to prevent or treat the disorder in question. The effective amount of such other agents depends on the amount of the checkpoint inhibitor present in the formulation, the type of disorder or treatment, and other factors discussed above, These are generally used in the same dosages and with administration routes as described herein, or about from 1 to 99% of the dosages described herein, or in any dosage and by any route that is empirically/clinically determined to be appropriate.

The progress of this therapy is easily monitored by conventional techniques and assays. For example, as a general proposition, the therapeutically effective amount of an immune checkpoint inhibitor, for example, a PD-L1 axis binding antagonist antibody, an anti-CTLA-4 antibody, an anti-IM-3 antibody, or an anti-LAG-3 antibody, administered to human will be in the range of about 0.01 to about 50 mg/kg of patient body weight, whether by one or more administrations. In some instances, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 1 5 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some instances, the antibody is administered at 15 mg/kg. However, other dosage regimens may be useful. In one instance, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21 -day cycles (every three weeks, q3w). In some instances, anti-PD-L1 antibody MPDL3280A is administered at 1200 mg intravenously every three weeks (q3w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some embodiments, the methods further involve administering to the patient an effective amount of an additional therapeutic agent. In some embodiments, the additional anti-cancer therapy comprises one or more of surgery, radiotherapy, chemotherapy, anti-angiogenic therapy, anti-DNA repair therapy, and anti-inflammatory therapy. In some instances, the additional therapeutic agent is selected from the group consisting of an anti-neoplastic; agent, a chemotherapeutic agent, a growth inhibitory agent, an anti-angiogenic agent, a radiation therapy, a cytotoxic agent, and combinations thereof. In some instances, a cancer immunotherapy may be administered in conjunction with a chemotherapy or chemotherapeutic agent. In some embodiments, the chemotherapy or chemotherapeutic agent is a platinum-based agent (including without limitation cisplatin, carboplatin, oxaliplatin, and staraplatin). In some instances, a cancer immunotherapy may be administered in conjunction with a radiation therapy agent. In some instances, a cancer immunotherapy may be administered in conjunction with a targeted therapy or targeted therapeutic agent. In some instances, a cancer immunotherapy may be administered in conjunction with another immunotherapy or immunotherapeutic agent, for example a monoclonal antibody. In some instances, the additional therapeutic agent is an agonist directed against a co-stimulatory molecule. In some instances, the additional therapeutic agent is an antagonist directed against a co-inhibitory molecule. In some instances, the cancer immunotherapy is administered as a monotherapy.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishes such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabien, navelbine, famesyl-protein tansferase inhibitors, transplatinum, and pharmaceutically acceptable salts, acids, or derivatives of any of the above.

Some (non-limiting) examples of chemotherapeutic drugs which can be combined with the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).” “Another large group of potential targets for complementary cancer therapy comprises kinase inhibitors, because the growth and survival of cancer cells is closely interlocked with the deregulation of kinase activity. To restore normal kinase activity and therefor reduce tumor growth a broad range of inhibitors is in used. The group of targeted kinases comprises receptor tyrosine kinases e.g. BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, ALK, cytoplasmic tyrosine kinases e.g. c-SRC, c-YES, Abl, JAK-2, serine/threonine kinases e.g. ATM, Aurora A & B, CDKs, mTOR, PKCi, PLKs, b-Raf, S6K, STK1 1/LKB1 and lipid kinases e.g. PI3K, SKI. Small molecule kinase inhibitors are e.g. PHA-739358, Nilotinib, Dasatinib, and PD166326, NSC 74341 1, Lapatinib (GW-572016), Canertinib (CI-1033), Semaxinib (SU5416), Vatalanib (PTK787/ZK222584), Sutent (SU1 1248), Sorafenib (BAY 43-9006) and Leflunomide (SU101). For more information see e.g. Zhang et al. 2009: Targeting cancer with small molecule kinase inhibitors. Nature Reviews Cancer 9, 28-39.” “Small molecule targeted therapy drugs are generally inhibitors of enzymatic domains on mutated, overexpressed, or otherwise critical proteins within the cancer cell. Prominent and nonlimiting examples are the tyrosine kinase inhibitors imatinib (Gleevec/Glivec) and gefitinib (Iressa).

In some embodiments, the additional anti-cancer therapy comprises anti-angiogenic therapy. Angiogenesis inhibitors prevent the extensive growth of blood vessels (angiogenesis) that tumors require to survive. The angiogenesis promoted by tumor cells to meet their increasing nutrient and oxygen demands for example can be blocked by targeting different molecules. Non-limiting examples of angiogenesis-mediating molecules or angiogenesis inhibitors which may be combined with the present invention are soluble VEGF (VEGF isoforms VEGF121 and VEGF165, receptors VEGFR1, VEGFR2 and co-receptors Neuropilin-1 and Neuropilin-2) 1 and NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, -β and -γ, CXCL10, IL-4, -12 and -18, prothrombin (kringle domain-2), antithrombin III fragment, prolactin, VEGI, SPARC, osteopontin, maspin, canstatin, proliferin-related protein, restin and drugs like e.g. bevacizumab, itraconazole, carboxyamidotriazole, TNP-470, CM101, IFN-α, platelet factor-4, suramin, SU5416, thrombospondin, VEGFR antagonists, angiostatic steroids+heparin, cartilage-derived angiogenesis Inhibitory factor, matrix metalloproteinase inhibitors, 2-methoxyestradiol, tecogalan, tetrathiomolybdate, thalidomide, thrombospondin, prolactina vβ3 inhibitors, linomide, and tasquinimod. In some embodiments, known therapeutic candidates include naturally occurring angiogenic inhibitors, including without limitation, angiostatin, endostatin, and platelet factor-4. In another embodiment therapeutic candidates include, without limitation, specific inhibitors of endothelial cell growth, such as TNP-470, thalidomide, and interleukin-12. Still other anti-angiogenic agents include those that neutralize angiogenic molecules, such as including without limitation, antibodies to fibroblast growth factor or antibodies to vascular endothelial growth factor or antibodies to platelet derived growth factor or antibodies or other types of inhibitors of the receptors of EGF, VEGF or PDGF. In some embodiments, antiangiogenic agents include without limitation suramin and its analogs, and tecogalan. In other embodiments, anti-angiogenic agents include without limitation agents that neutralize receptors for angiogenic factors or agents that interfere with vascular basement membrane and extracellular matrix, including, without limitation, metalloprotease inhibitors and angiostatic steroids. Another group of anti-angiogenic compounds includes, without limitation, anti-adhesion molecules, such as antibodies to integrin alpha v beta 3. Still other anti-angiogenic compounds or compositions, include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound is an antibody to VEGF, such as Avastin®/bevacizumab (Genentech).

In some embodiments, the additional anti-cancer therapy comprises anti-DNA repair therapy. In some embodiments, the DNA damage repair and response inhibitor is selected from a PARP inhibitor, a RAD51 inhibitor, or an inhibitor of a DNA damage response kinase selected from CHCK1, ATM, or ATR. In some embodiments, the additional anti-cancer therapy comprises a radiosensitizer. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. SSB repair mechanisms include BER, NER, or MMR pathways whilst DSB repair mechanisms consist of HR and NHEJ pathways. Radiation causes DNA breaks that if not repaired are lethal. Single strand breaks are repaired through a combination of BER, NER and MMR mechanisms using the intact DNA strand as a template. The predominant pathway of SSB repair is the BER utilizing a family of related enzymes termed poly-(ADP-ribose) polymerases (PARP). Thus, the radiosensitizer can include DNA damage response inhibitors such as Poly (ADP) ribose polymerase (PARP) inhibitors. In some embodiments, the additional anti-cancer therapy is a DNA repair and response pathway inhibitor, PARP inhibitor (e.g., Talazoparib, Rucaparib, Olaparib), RAD51 inhibitor (RI-1), or an inhibitor of DNA damage response kinases such as CHCK1 (AZD7762), ATM (KU-55933, KU-60019, NU7026, VE-821), and ATR (NU7026).

In some embodiments, the additional anti-cancer therapy comprises anti-inflammatory agent. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23, interferons (IFNs), e.g., IFNα, IFNβ, IFNγ, IFN-γ inducing factor (IGIF), transforming growth factor-β (TGF-β), transforming growth factor-α (TGF-α), tumor necrosis factors TNF-α, TNF-β, TNF-RI, TNF-RII, CD23, CD30, CD40L, EGF, G-CSF, GDNF, PDGF-BB, RANTES/CCL5, IKK, NF-κB, TLR2, TLR3, TLR4, TL5, TLR6, TLR7, TLR8, TLR8, TLR9, and/or any cognate receptors thereof. In some embodiments, the anti-inflammatory agent is an IL-1 or IL-1 receptor antagonist, such as anakinra (KINERET®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (REMICADE®), golimumab (SIMPONI®), adalimumab (HUMIRA®), certolizumab pegol (CIMZIA®) or etanercept.

In some embodiments, the anti-inflammatory agent is a corticosteroid. Exemplary corticosteroids include, but are not limited to, cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PREZONE®), and prednisone (DELTASONE®, LIQUID PRED®, METICORTEN®, ORASONE®)), and bisphosphonates (e.g., pamidronate (AREDIA®), and zoledronic acid (ZOMETAC®).

Such combination therapies noted above encompass combined administration (where two or more therapeutic agents are included in the same or separate formulations), and separate administration, in which case, administration of a cancer immunotherapy can occur prior to, simultaneously, and/or following, administration of the additional therapeutic agent or agents. In one instance, administration of a cancer immunotherapy and administration of an additional ⁻therapeutic agent occur within about one month, or within about one, two or three weeks, or within about one, two, three, four, five, or six days, of each other.

Without wishing to be bound to theory, it is thought that enhancing T-cell stimulation, by promoting a co-stimulatory molecule or by inhibiting a co-inhibitory molecule, may promote tumor cell death thereby treating or delaying progression of cancer. In some instances, an immune checkpoint inhibitor, for example, a PD-L1 axis binding antagonist and/or CTLA4 antagonist, may be administered in conjunction with an agonist directed against a co-stimulatory molecule. In some instances, a co-stimulatory molecule may include CD40, CD226, CD28, OX40, GITR, CD137, CD27, HVEM, or CD127. In some instances, the agonist directed against a co-stimulatory molecule is an agonist antibody that binds to CD40, CD226, CD28, OX40, GITR, CD137, CD27, HVEM, or CD127. In some instances, an immune checkpoint inhibitor, for example, a PD-L1 axis binding antagonist and/or CTLA4 antagonist, may be administered in conjunction with an antagonist directed against a co-inhibitory molecule. In some instances, a co-inhibitory molecule may include CTLA-4 (also known as CD1 52), TIM-3, BTLA, VISTA, LAG-3, B7-H3, B7-H4, IDO, TIGIT, MICA/B, or arginase. In some instances, the antagonist directed against a co-inhibitory molecule is an antagonist antibody that binds to CTLA-4, T1M-3, BTLA, VISTA, LAG-3, B7-H3, B7-H4, IDO, TIGIT. MICA/B, or arginase.

In some instances, a PD-L1 axis binding antagonist may be administered in conjunction with an antagonist directed against CTLA-4 (also known as CD152), e.g., a blocking antibody. In some instances, a PD-L1 axis binding antagonist may be administered in conjunction with ipilimumab (also known as MDX-010, MDX-101 , or YERVOY®), In some instances, a PD-L1 axis binding antagonist may be administered in conjunction with tremelimumab (also known as ticilimumab or CP-675,206), In some instances, a PD-L1 axis binding antagonist may be administered in conjunction with an antagonist directed against B7-H3 (also known as CD276), e.g., a blocking antibody, In some instances, a PD-L1 axis binding antagonist may be administered in conjunction with MGA27i . In some instances, a PD-L1 axis binding antagonist may be administered in conjunction with an antagonist directed against a TGF-beta, e.g., metelimumab (also known as CAT-192), fresolimumab (also known as (IC1008), or LY2157299.

In some instances, an immune checkpoint inhibitor, for example, a PD-L1 axis binding antagonist and/or CTLA4 antagonist, may be administered in conjunction with a treatment comprising adoptive transfer of a T-cell (e.g., a cytotoxic T-cell or CTL) expressing a chimeric antigen receptor (CAR). In some instances, an immune checkpoint inhibitor, for example, a PD-L1 axis binding antagonist and/or CTLA4 antagonist, may be administered in conjunction with a treatment comprising adoptive transfer of a T-cell comprising a dominant-negative TGF beta receptor, e.g., a dominant-negative TGF beta type II receptor. In some instances, an immune checkpoint inhibitor, for example, a PD-L1 axis binding antagonist and/or CTLA4 antagonist, may be administered in conjunction with a treatment comprising a HERCREEM protocol (see, e.g., ClinicalTrials.gov Identifier NCT00889954).

In some instances, an immune checkpoint inhibitor, for example, a PD-L1 axis binding antagonist and/or CTLA4 antagonist, may be administered in conjunction with an agonist directed against CD137 (also known as TNFRSF9, 4-1 BB, or ILA), e.g., an activating antibody. In some instances, an immune checkpoint inhibitor, for example, a PD-L1 axis binding antagonist and/or CTLA4 antagonist, may be administered in conjunction with urelumab (also known as BMS-663513). In some instances, an immune checkpoint inhibitor, for example, a PD-L1 axis binding antagonist and/or CTLA4 antagonist, may be administered in conjunction with an agonist directed against CD40, e.g., an activating antibody. In some instances, an immune checkpoint inhibitor, for example, a PD-L1 axis binding antagonist and/or CTLA4 antagonist, may be administered in conjunction with CP-870893, In some instances, an immune checkpoint inhibitor, for example, a PD-L1 axis binding antagonist and/or CTLA4 antagonist, may be administered in conjunction with an agonist directed against OX40 (also known as CD134), e.g., an activating antibody. In some instances, an immune checkpoint inhibitor, for example, a PD-L1 axis binding antagonist and/or CTLA4 antagonist, may be administered in conjunction with an anti-OX40 antibody (e.g., AgonOX), In some instances, an immune checkpoint inhibitor, for example, a PD-L1 axis binding antagonist and/or CTLA4 antagonist, may be administered in conjunction with an agonist directed against CD27, e.g., an activating antibody. In some instances, an immune checkpoint inhibitor, for example, a PD-L1 axis binding antagonist and/or CTLA4 antagonist, may be administered in conjunction with CDX-1127. In some instances, an immune checkpoint inhibitor, for example, a PD-L1 axis binding antagonist and/or CTLA4 antagonist, may be administered in conjunction with an antagonist directed against indoleamine-2,3-dioxygenase (IDO). In some instances, with the IDO antagonist is 1-methyl-D-tryptophan (also known as 1-D-MT).

In some instances, an immune checkpoint inhibitor, for example, a PD-L1 axis binding antagonist and/or CTLA4 antagonist, may be administered in conjunction with an antibody-drug conjugate. In some instances, the antibody-drug conjugate comprises mertansine or monomethyl auristatin E (MMAE). In some instances, an immune checkpoint inhibitor, for example, a PD-L1 axis binding antagonist and/or CTLA4 antagonist, may be administered in conjunction with an anti-NaPi2b antibody-MMAE conjugate (also known as DNIB0600A or RG7599). In some instances, an immune checkpoint inhibitor, for example, a PD-L1 axis binding antagonist and/or CTLA4 antagonist, may be administered in conjunction with trastuzumab enrtansine (also known as T-DM1, ado-trastuzumab emtansine, or KADCYLA®, Genentech). In some instances, an immune checkpoint inhibitor, for example, a PD-L1 axis binding antagonist and/or CTLA4 antagonist, may be administered in conjunction with DMUC5754A. In some instances, an immune checkpoint inhibitor, for example, a PD-L1 axis binding antagonist and/or CTLA4 antagonist, may be administered in conjunction with an antibody-drug conjugate targeting the endothelin B receptor (EDNBR), e.g., an antibody directed against EDNBR conjugated with MMAE.

In some instances, an immune checkpoint inhibitor, for example, a PD-L1 axis binding antagonist and/or CTLA4 antagonist, may be administered in conjunction with an anti-angiogenesis agent. In some instances, an immune checkpoint inhibitor, for example, a PD-L1 axis binding antagonist and/or CTLA4 antagonist, may be administered in conjunction with an antibody directed against a VEGF, VEGF-A. In some instances, an immune checkpoint inhibitor, for example, a PD-L1 axis binding antagonist and/or CTLA4 antagonist, may be administered in conjunction with bevacizumab (also known as AVASTIN®, Genentech). In some instances, an immune checkpoint inhibitor, for example, a PD-L1 axis binding antagonist and/or CTLA4 antagonist, may be administered in conjunction with an antibody directed against angiopoietin 2 (also known as Ang2). In some instances, an immune checkpoint inhibitor, for example, a PD-L1 axis binding antagonist and/or CTLA4 antagonist, may be administered in conjunction with MEDI3617.

In some instances, an immune checkpoint inhibitor, for example, a PD-L1 axis binding antagonist and/or CTLA4 antagonist, may be administered in conjunction with an antineoplastic agent. In some instances, an immune checkpoint inhibitor, for example, a PD-L1 axis binding antagonist and/or CTLA4 antagonist, may be administered in conjunction with an agent targeting CSF-1 R (also known as M-CSFR or CD1 15). In some instances, an immune checkpoint inhibitor, for example, a PD-L1 axis binding antagonist and/or CTLA4 antagonist, may be administered in conjunction with anti-CSF-1 R (also known as IMC-CS4). In some instances, an immune checkpoint inhibitor, for example, a PD-L1 axis binding antagonist and/or CTLA4 antagonist, may be administered in conjunction with an interferon, for example interferon alpha or interferon gamma. In some instances, an immune checkpoint inhibitor, for example, a PD-L1 axis binding antagonist and/or CTLA4 antagonist, may be administered in conjunction with Roferon-A (also known as recombinant Interferon alpha-2a). In some instances, an immune checkpoint inhibitor, for example, a PD-L1 axis binding antagonist and/or CTLA4 antagonist, may be administered in conjunction with GM-CSF (also known as recombinant human granulocyte macrophage colony stimulating factor, rhu GM-CSF, sargramostim, or LEUKINE®). In some instances, an immune checkpoint inhibitor, for example, a PD-L1 axis binding antagonist and/or CTLA4 antagonist, may be administered in conjunction with IL-2 (also known as aldesleukin or PROLEUKIN®). In some instances, an immune checkpoint inhibitor, for example, a PD-L1 axis binding antagonist and/or CTLA4 antagonist, may be administered in conjunction with IL-12. In some instances, an immune checkpoint inhibitor, for example, a PD-L1 axis binding antagonist and/or CTLA4 antagonist, may be administered in conjunction with an antibody targeting CD20. In some instances, the antibody targeting CD20 is obinutuzumab (also known as GA101 or GAZYVA®) or rituximab. In some instances, an immune checkpoint inhibitor, for example, a PD-L1 axis binding antagonist and/or CTLA4 antagonist, may be administered in conjunction with an antibody targeting GIRR. In some instances, the antibody targeting GITR is TRX518.

In some instances, an immune checkpoint inhibitor, for example, a PD-L1 axis binding antagonist and/or CTLA4 antagonist, may be administered in conjunction with a cancer vaccine. In some instances, the cancer vaccine is a peptide cancer vaccine, which in some instances is a personalized peptide vaccine. In some instances the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, an immune checkpoint inhibitor, for example, a PD-L1 axis binding antagonist and/or CTLA4 antagonist, may be administered in conjunction with an adjuvant. In some instances, an immune checkpoint inhibitor, for example, a PD-L1 axis binding antagonist and/or CTLA4 antagonist, may be administered in conjunction with a treatment comprising a TLR agonist, e.g., Poly-ICLC (also known as HILTONOL®), LPS, MPL, or CpG ODN. In some instances, an immune checkpoint inhibitor, for example, a PD-L1 axis binding antagonist and/or CTLA4 antagonist, may be administered in conjunction with tumor necrosis factor (TNF) alpha. In some instances, an immune checkpoint inhibitor, for example, a PD-L1 axis binding antagonist and/or CTLA4 antagonist, may be administered in conjunction with IL-1. In some instances, an immune checkpoint inhibitor, for example, a PD-L1 axis binding antagonist and/or CTLA4 antagonist, may be administered in conjunction with HMGB1. In some instances, an immune checkpoint inhibitor, for example, a PD-L1 axis binding antagonist and/or CILA4 antagonist, may be administered in conjunction with an IL-10 antagonist. In some instances, an immune checkpoint inhibitor, for example, a PD-L1 axis binding antagonist and/or CTLA4 antagonist, may be administered in conjunction with an IL-4 antagonist. In some instances, an immune checkpoint inhibitor, for example, a PD-L1 axis binding antagonist and/or CTLA4 antagonist, may be administered in conjunction with an IL-13 antagonist. In some instances, an immune checkpoint inhibitor, for example, a PD-L1 axis binding antagonist and/or CTLA4 antagonist, may be administered in conjunction with an HVEM antagonist. In some instances, an immune checkpoint inhibitor, for example, a PD-L1 axis binding antagonist and/or CTLA4 antagonist, may be administered in conjunction with an ICOS agonist, e.g., by administration of ICOS-L, or an agonistic antibody directed against ICOS. In some instances, an immune checkpoint inhibitor, for example, a PD-L1 axis binding antagonist and/or CTLA4 antagonist, may be administered in conjunction with a treatment targeting CX3CL1. In some instances, an immune checkpoint inhibitor, for example, a PD-L1 axis binding antagonist and/or CTLA4 antagonist, may be administered in conjunction with a treatment targeting CXCL9. In some instances, an immune checkpoint inhibitor, for example, a PD-L1 axis binding antagonist and/or CTLA4 antagonist, may be administered in conjunction with a treatment targeting CXCL10. In some instances, an immune checkpoint inhibitor, for example, a PD-L1 axis binding antagonist and/or CTLA4 antagonist, may be administered in conjunction with a treatment targeting CCL5. In some instances, an immune checkpoint inhibitor, for example, a PD-L1 axis binding antagonist and/or CTLA4 antagonist, may be administered in conjunction with an LFA-1 or ICAM1 agonist. In some instances, an immune checkpoint inhibitor, for example, a PD-L1 axis binding antagonist and/or CTLA4 antagonist, may be administered in conjunction with a Selectin agonist.

In some instances, an immune checkpoint inhibitor, for example, a PD-L1 axis binding antagonist and/or CTLA4 antagonist, may be administered in conjunction with a targeted therapy. In some instances, an immune checkpoint inhibitor, for example, a PD-L1 axis binding antagonist and/or CTLA4 antagonist, may be administered in conjunction with an inhibitor of B-Raf. In some instances, an immune checkpoint inhibitor, for example, a PD-L1 axis binding antagonist and/or CTLA4 antagonist, may be administered in conjunction with vemurafenib (also known as ZELBORAF®). In some instances, an immune checkpoint inhibitor, for example, a PD-L1 axis binding antagonist ane/or CTLA4 antagonist, may be administered in conjunction with dabrafenib (also known as TAFINLAR®). In some instances, an immune checkpoint inhibitor, for example, a PD-L1 axis binding antagonist and/or CTLA4 antagonist, may be administered in conjunction with erlotinib (also known as TARCEVA®). In some instances, an immune checkpoint inhibitor, for example, a PD-L1 axis binding antagonist and/or CTLA4 antagonist, may be administered in conjunction with an inhibitor of a MEK, such as MEK1 (also known as MAP2K1) or MFK2 (also known as MAP2K2). In some instances, an immune checkpoint inhibitor, for example, a PD-L1 axis binding antagonist and/or CTLA4 antagonist, may be administered in conjunction with cobimetinib (also known as GDC-0973 or XL-518), In some instances, an immune checkpoint inhibitor, for example, a PD-L1 axis binding antagonist and/or CTLA4 antagonist, may be administered in conjunction with trametinib (also known as MEKIMST®), In some instances, an immune checkpoint inhibitor, for example, a PD-L1 axis binding antagonist and/or CTLA4 antagonist, may be administered in conjunction with an inhibitor of K-Ras. In some instances, an immune checkpoint inhibitor, for example, a PD-L1 axis binding antagonist and/or CTLA4 antagonist, may be administered in conjunction with an inhibitor of c-Met. In some instances, an immune checkpoint inhibitor, for example, a PD-L1 axis binding antagonist and/or CTLA4 antagonist, may be administered in conjunction with onartuzumab (also known as MetMAb). In some instances, an immune checkpoint inhibitor, for example, a PD-L1 axis binding antagonist and/or CTLA4 antagonist, may be administered in conjunction with an inhibitor of Alk. In some instances, an immune checkpoint inhibitor, for example, a PD-L1 axis binding antagonist and/or CTLA4 antagonist, may be administered in conjunction with AF802 (also known as CH5424802 or alectinib). In some instances, an immune checkpoint inhibitor, for example, a PD-L1 axis binding antagonist and/or CTLA4 antagonist, may be administered in conjunction with an inhibitor of a phosphatidylinositol 3-kinase (PI1K). In some instances, an immune checkpoint inhibitor, for example, a PD-L1 axis binding antagonist and/or CTLA4 antagonist, may be administered in conjunction with BKM120.

In some instances, an immune checkpoint inhibitor, for example, a PD-L1 axis binding antagonist and/or CTLA4 antagonist, may be administered in conjunction with idelalisib (also known as GS-1101 or CAL-101). In some instances, an immune checkpoint inhibitor, for example, a PD-L1 axis binding antagonist and/or CTLA4 antagonist, may be administered in conjunction with perifosine (also known as KRX-0401). In some instances, an immune checkpoint inhibitor, for example, a PD-L1 axis binding antagonist and/or CTLA4 antagonist, may be administered in conjunction with an inhibitor of an Akt. In some instances, an immune checkpoint inhibitor, for example, a PD-L1 axis binding antagonist and/or CTLA4 antagonist, may be administered in conjunction with MK2206. In some instances, an immune checkpoint inhibitor, for example, a PD-L1 axis binding antagonist and/or CTLA4 antagonist, may be administered in conjunction with GSK690693. In some instances, an immune checkpoint inhibitor, for example, a PD-L1 axis binding antagonist and/or CTLA4 antagonist, may be administered in conjunction with GDC-0941. In some instances, an immune checkpoint inhibitor, for example, a PD-L1 axis binding antagonist and/or CTLA4 antagonist, may be administered in conjunction with an inhibitor of mTOR. In some instances, an immune checkpoint inhibitor, for example, a PD-L1 axis binding antagonist and/or CTLA4 antagonist, may be administered in conjunction with sirolimus (also known as rapamycin). In some instances, an immune checkpoint inhibitor, for example, a PD-L1 axis binding antagonist and/or CTLA4 antagonist, may be administered in conjunction with temsirolimus (also known as CCI-779 or TORISEL®). In some instances, an immune checkpoint inhibitor, for example, a PD-L1 axis binding antagonist and/or CTLA4 antagonist, may be administered in conjunction with everolimus (also known as RAD001). In some instances, an immune checkpoint inhibitor, for example, a PD-L1 axis binding antagonist and/or CTLA4 antagonist, may be administered in conjunction with ridaforolimus (also known as AP-23573, MK-8669, or deforolimus). In some instances, an immune checkpoint inhibitor, for example, a PD-L1 axis binding antagonist and/or CTLA4 antagonist, may be administered in conjunction with OSI-027. In some instances, an immune checkpoint inhibitor, for example, a PD-L1 axis binding antagonist and/or CTLA4 antagonist, may be administered in conjunction with AZD8055. In some instances, an immune checkpoint inhibitor, for example, a PD-L1 axis binding antagonist and/or CTLA4 antagonist, may be administered in conjunction with INK128. In some instances, an immune checkpoint inhibitor, for example, a PD-L1 axis binding antagonist and/or CTLA4 antagonist, may be administered in conjunction with a dual PI3K/mTOR inhibitor. In some instances, an immune checkpoint inhibitor, for example, a PD-L1 axis binding antagonist and/or CTLA4 antagonist, may be administered in conjunction with XL765. In some instances, an immune checkpoint inhibitor, for example, a PD-L1 axis binding antagonist and/or CTLA4 antagonist, may be administered in conjunction with GDC-0980. In some instances, an immune checkpoint inhibitor, for example, a PD-L1 axis binding antagonist and/or CTLA4 antagonist, may be administered in conjunction with BEZ235 (also known as NVP-BEZ235). In some instances, an immune checkpoint inhibitor, for example, a PD-L1 axis binding antagonist and/or CTLA4 antagonist, may be administered in conjunction with BGT226. In some instances, an immune checkpoint inhibitor, for example, a PD-L1 axis binding antagonist and/or CTLA4 antagonist, may be administered in conjunction with GSK2126458. In some instances, an immune checkpoint inhibitor, for example, a PD-L1 axis binding antagonist and/or CTLA4 antagonist, may be administered in conjunction with PF-04691502. In some instances, an immune checkpoint inhibitor, for example, a PD-L1 axis binding antagonist and/or CTLA4 antagonist, may be administered in conjunction with PF-05212384 (also known as PM-587).

While PD-L1 axis binding antagonists and CTLA4 antagonists are called out supra as exemplary cancer immunotherapies, this is not intended to be limiting; any cancer immunotherapy of the present disclosure may be administered in conjunction with any of the other treatments described herein, or otherwise known in the art (subject to medical judgement).

### Computer Systems

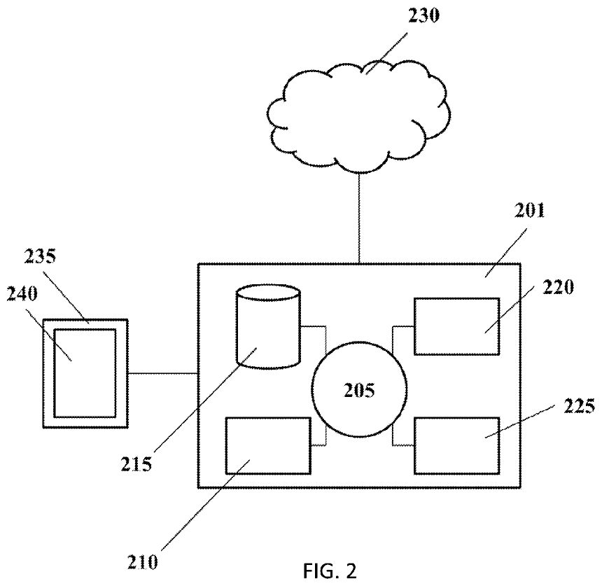

The present disclosure provides computer systems that are programmed to implement methods of the disclosure. FIG. 2 shows a computer system 201 that is programmed or otherwise configured to, for example, process WGS data to determine copy number aberrations (CNAs) and fragment lengths of cfDNA molecules, process CNAs to determine a CNA profile change, process fragment lengths to determine a fragment length profile change, process MS data to determine an average methylation fraction for each of one or more CpG islands in a region of a genome, process average methylation fraction profiles across CpG islands to determine methylation fraction profiles, determine a tumor fraction of the subject at a timepoint, and detect a tumor progression of the subject. The computer system 201 can regulate various aspects of analysis, calculation, and generation of the present disclosure, such as, for example, processing WGS data to determine copy number aberrations (CNAs) and fragment lengths of cfDNA molecules, processing CNAs to determine a CNA profile change, processing fragment lengths to determine a fragment length profile change, processing MS data to determine an average methylation fraction for each of one or more CpG islands in a region of a genome, processing average methylation fraction profiles across CpG islands to determine methylation fraction profiles, determining a tumor fraction of the subject at a timepoint, and detecting a tumor progression of the subject. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), to form a motherboard. The storage unit 215 can be a data storage unit (or data repository) for storing data. The computer system 201 can be operatively coupled to a computer network (“network”) 230 with the aid of the communication interface 220. The network 230 can be an Internet, or an internet and/or extranet, that is in communication with an Internet. The network 230 in some cases is a telecommunication and/or data network. In some embodiments, network 230 can include a local area network (“LAN”), including without limitation an Ethernet network, a Token-Ring network and/or the like; a wide-area network; a wireless wide area network (“WWAN”); a virtual network, such as a virtual private network (“VPN”); the Internet; an intranet; an extranet; a wireless network, including without limitation a network operating under any of the IEEE 802.11 suite of protocols, the BluetoothTM protocol known in the art, and/or any other wireless protocol; and/or any combination of these and/or other networks. The network 230 can include one or more computer servers, which can enable distributed computing, such as cloud computing. For example, one or more computer servers may enable cloud computing over the network 230 (“the cloud”) to perform various aspects of analysis, calculation, and generation of the present disclosure, such as, for example, processing WGS data to determine copy number aberrations (CNAs) and fragment lengths of cfDNA molecules, processing CNAs to determine a CNA profile change, processing fragment lengths to determine a fragment length profile change, processing MS data to determine an average methylation fraction for each of one or more CpG islands in a region of a genome, processing average methylation fraction profiles across CpG islands to determine methylation fraction profiles, determining a tumor fraction of the subject at a timepoint, and detecting a tumor progression of the subject. Such cloud computing may be provided by cloud computing platforms such as, for example, Amazon® Web Services (AWS), Microsoft® Azure, Google® Cloud Platform, and IBM® cloud. The network 230, in some cases with the aid of the computer system 201, can implement a peer-to-peer network, which may enable devices coupled to the computer system 201 to behave as a client or a server.

The CPU 205 can execute a sequence of machine-readable instructions, stored on memory 210, which can be embodied in a program or software. The instructions are executed by the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC), microprocessor, core, or memory chip. It should be appreciated that the CPU can be any type of electronic circuitry.

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., a physician, a nurse, a caretaker, a patient, or a subject). Examples of remote computer systems include personal computers (e.g., portable PC), slate or tablet PC's (e.g., Apple® iPad, Samsung® Galaxy Tab), telephones, Smart phones (e.g., Apple® iPhone, Android®-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, determined CNAs and fragment lengths of cfDNA molecules, determined CNA profile changes, determined fragment length profile changes, determined tumor fractions, detected tumor progression or non-progression of the subject, detected tumor status (e.g., progression or non-progression), tumor progression/tumor non-progression status over time (e.g., provided numerically or plotted), determined methylation status or changes thereto, and the like. Examples of UI's include, without limitation, a graphical user interface (GUI). A GUI could include, without limitation, a web-based user interface or an application based user interface for execution on a mobile device.

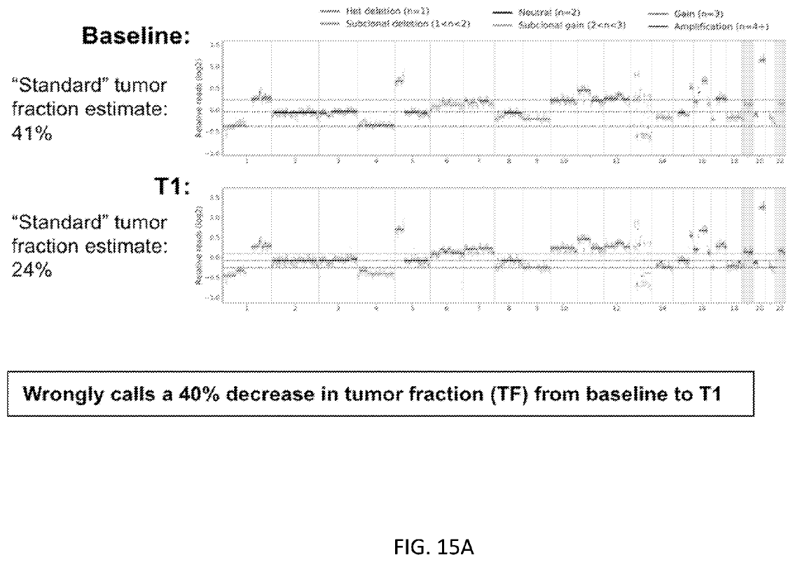

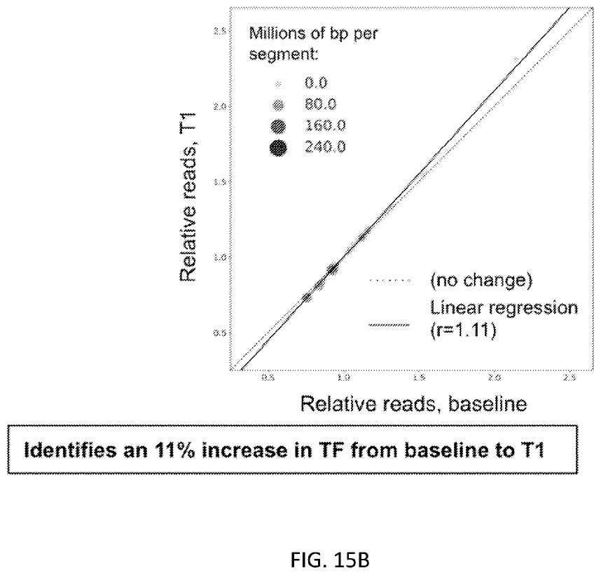

Methods and systems of the present disclosure can be implemented by way of one or more algorithms. An algorithm can be implemented by way of software upon execution by the central processing unit 205. The algorithm can, for example, process WGS data to determine copy number aberrations (CNAs) and fragment lengths of cfDNA molecules, process CNAs to determine a CNA profile change, process fragment lengths to determine a fragment length profile change by quantifying the change in strength of a specific CNA signal in multiple samples from the patient over the course of treatment (which were shown to be less prone to certain error modes arising from separately quantifying tumor fractions in separate samples based on CNAs, see FIGS. 15A and 15B), process MS data to determine an average methylation fraction for each of one or more CpG islands in a region of a genome, process average methylation fraction profiles across CpG islands to determine methylation fraction profiles, determine a tumor fraction of the subject at a timepoint based on training on orthogonal data, and detect a tumor progression of the subject.

Although this is described as a statistical modeling technique, the above processes could also be carried out by modifying various well-known machine learning techniques.

### Enumerated Embodiments

The following enumerated embodiments are representative of some aspects of the invention.
1. A method for assessing tumor status of a subject with cancer, comprising:

obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;

determining, based on the first WGS data, (i) a first plurality of copy number aberrations (CNAs) in the first plurality of cfDNA molecules and (ii) a first plurality of fragment lengths of the first plurality of cfDNA molecules;

obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;

determining, based on the second WGS data, (iii) a second plurality of copy number aberrations (CNAs) in the second plurality of cfDNA molecules and (iv) a second plurality of fragment lengths of the second plurality of cfDNA molecules;

comparing the first plurality of CNAs with the second plurality of CNAs to determine a CNA profile change;

determining a fragment length profile change based on the first plurality of fragment lengths and the second plurality of fragment lengths;

determining a first tumor fraction of the subject at the first timepoint or a second tumor fraction of the subject at the second timepoint, based at least in part on the CNA profile change and the fragment length profile change; and

detecting a tumor status of the subject based at least in part on the first tumor fraction or the second tumor fraction.
2. The method of embodiment 1, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.
3. The method of embodiment 1, wherein obtaining the first WGS data comprises sequencing the first plurality of cfDNA molecules to generate a first plurality of sequencing reads, or wherein obtaining the second WGS data comprises sequencing the second plurality of cfDNA molecules to generate a second plurality of sequencing reads.
4. The method of embodiment 3, wherein the sequencing is performed at a depth of no more than about 25×.
5. The method of embodiment 3, wherein the sequencing is performed at a depth of no more than about 10×.
6. The method of embodiment 3, wherein the sequencing is performed at a depth of no more than about 8×.
7. The method of embodiment 3, wherein the sequencing is performed at a depth of no more than about 6×.
8. The method of embodiment 3, further comprising aligning the first or second plurality of sequencing reads to a reference genome, thereby producing a plurality of aligned sequencing reads.
9. The method of embodiment 1, further comprising enriching the first or second plurality of cfDNA molecules for a plurality of genomic regions.
10. The method of embodiment 9, wherein the enrichment comprises amplifying the first or second plurality of cfDNA molecules.
11. The method of embodiment 10, wherein the amplification comprises selective amplification.
12. The method of embodiment 10, wherein the amplification comprises universal amplification.
13. The method of embodiment 9, wherein the enrichment comprises selectively isolating at least a portion of the first or second plurality of cfDNA molecules.
14. The method of embodiment 13, wherein selectively isolating the at least the portion of the first or second plurality of cfDNA molecules comprises using a plurality of probes, each of the plurality of probes having sequence complementarity with at least a portion of a genomic region of the plurality of genomic regions.
15. The method of embodiment 13, wherein the at least the portion comprises a tumor marker locus.
16. The method of embodiment 15, wherein the at least the portion comprises a plurality of tumor marker loci.
17. The method of embodiment 16, wherein the plurality of tumor marker loci comprises one or more loci selected from The Cancer Genome Atlas (TCGA) or Catalogue of Somatic Mutations in cancer (COSMIC).
18. The method of embodiment 3, wherein determining the first plurality of CNAs comprises determining quantitative measures of CNAs at each of at each of a plurality of genomic regions of the first plurality of sequencing reads, and wherein determining the second plurality of CNAs comprises determining quantitative measures of CNAs at each of at each of the plurality of genomic regions of the second plurality of sequencing reads.
19. The method of embodiment 18, further comprising correcting the first plurality of CNAs or the second plurality of CNAs for GC content and/or mappability bias.
20. The method of embodiment 19, wherein the correcting comprises using a statistical modeling analysis.
21. The method of embodiment 20, wherein the statistical modeling analysis comprises LOESS regression or a Bayesian model.
22. The method of embodiment 18, wherein the plurality of genomic regions comprises non-overlapping genomic regions of a reference genome having a pre-determined size.
23. The method of embodiment 22, wherein the pre-determined size is about 50 kilobases (kb), about 100 kb, about 200 kb, about 500 kb, about 1 megabases (Mb), about 2 Mb, about 5 Mb, or about 10 Mb.
24. The method of embodiment 18, wherein the plurality of genomic regions comprises at least about 1,000 distinct genomic regions.
25. The method of embodiment 24, wherein the plurality of genomic regions comprises at least about 2,000 distinct genomic regions.
26. The method of embodiment 1, wherein determining the CNA profile change comprises comparing the first plurality of CNAs and the second plurality of CNAs with a plurality of reference CNA values, wherein the plurality of reference CNA values is obtained from additional cfDNA molecules obtained or derived from additional bodily fluid samples of additional subjects.
27. The method of embodiment 26, wherein the additional subjects comprise one or more subjects without cancer.
28. The method of embodiment 26, wherein the additional subjects comprise one or more subjects not having tumor progression.
29. The method of embodiment 26, wherein the plurality of reference CNA values is obtained using additional bodily fluid samples of the subject obtained at one or more subsequent time points after the first timepoint.
30. The method of embodiment 1, further comprising filtering out a subset of the first plurality of CNAs and the second plurality of CNAs that meet a pre-determined criterion.
31. The method of embodiment 30, further comprising filtering out a given CNA value of the first plurality of CNAs or the second plurality of CNAs values when the difference between the given CNA value and the corresponding reference CNA value comprises a difference of no more than about 1 standard deviation.
32. The method of embodiment 31, further comprising filtering out a given CNA value of the first plurality of CNAs or the second plurality of CNAs values when the difference between the given CNA value and the corresponding reference CNA value comprises a difference of no more than about 2 standard deviations.
33. The method of embodiment 31, further comprising filtering out a given CNA value of the first plurality of CNAs or the second plurality of CNAs values when the difference between the given CNA value and the corresponding reference CNA value comprises a difference of no more than about 3 standard deviations.
34. The method of embodiment 30, further comprising filtering out a given CNA value of the first plurality of CNAs or the second plurality of CNAs values based on a Spearman's rank correlation between the given CNA value and a corresponding local mean fragment length.
35. The method of embodiment 34, further comprising filtering out a given CNA value of the first plurality of CNAs or the second plurality of CNAs values when the Spearman's rank correlation coefficient (Spearman's rho) is less than -0.1.
36. The method of embodiment 1, further comprising normalizing the first plurality of fragment lengths or the second plurality of fragment lengths based on a library or a genomic location.
37. The method of embodiment 1, further comprising detecting that the tumor status comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5.
38. The method of embodiment 1, further comprising detecting a major molecular response (MMR) of the subject when the first tumor fraction or the second tumor fraction is less than 0.01, less than 0.05, less than 0.1, less than 0.2, less than 0.3, less than 0.4, or less than 0.5.
39. The method of any one of embodiments 1-38, further comprising detecting the tumor status of the subject with a sensitivity of at least about 50%.
40. The method of embodiment 39, further comprising detecting the tumor status of the subject with a sensitivity of at least about 70%.
41. The method of embodiment 40, further comprising detecting the tumor status of the subject with a sensitivity of at least about 90%.
42. The method of any one of embodiments 1-41, further comprising detecting the tumor status of the subject with a specificity of at least about 50%.
43. The method of embodiment 42, further comprising detecting the tumor status of the subject with a specificity of at least about 70%.
44. The method of embodiment 43, further comprising detecting the tumor status of the subject with a specificity of at least about 90%.
45. The method of embodiment 44, further comprising detecting the tumor status of the subject with a specificity of at least about 98%.
46. The method of any one of embodiments 1-45, further comprising detecting the tumor status of the subject with a positive predictive value (PPV) of at least about 50%.
47. The method of embodiment 46, further comprising detecting the tumor status of the subject with a positive predictive value (PPV) of at least about 70%.
48. The method of embodiment 47, further comprising detecting the tumor status of the subject with a positive predictive value (PPV) of at least about 90%.
49. The method of any one of embodiments 1-48, further comprising detecting the tumor status of the subject with a negative predictive value (NPV) of at least about 50%.
50. The method of embodiment 49, further comprising detecting the tumor status of the subject with a negative predictive value (NPV) of at least about 70%.
51. The method of embodiment 50, further comprising detecting the tumor status of the subject with a negative predictive value (NPV) of at least about 90%.
52. The method of any one of embodiments 1-51, further comprising detecting the tumor status of the subject with an area under the curve (AUC) of at least about 0.60.
53. The method of embodiment 52, further comprising detecting the tumor status of the subject with an area under the curve (AUC) of at least about 0.75.
54. The method of embodiment 53, further comprising detecting the tumor status of the subject with an area under the curve (AUC) of at least about 0.90.
55. The method of any one of embodiments 1-54, further comprising determining a tumor non-progression of the subject when tumor progression is not detected.
56. The method of any one of embodiments 1-55, further comprising, based on the determined tumor status of the subject, administering a therapeutically effective dose of a treatment to treat the cancer of the subject.
57. The method of embodiment 56, wherein the treatment comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor.
58. The method of any one of embodiments 1-57, wherein the detected tumor status is indicative of tumor progression, non-progression, regression, or recurrence.
59. The method of any one of embodiments 1-58, wherein the first and second WGS data are obtained by pyrosequencing, sequencing-by-synthesis, single-molecule sequencing, Nanopore sequencing, semiconductor sequencing, sequencing-by-ligation, sequencing-by-hybridization, massively parallel sequencing, chain termination sequencing, single molecule real-time sequencing, Polony sequencing, combinatorial probe anchor synthesis, or hybrid capture-based sequencing.
60. The method of any one of embodiments 1-59, wherein the first and second WGS data are obtained by a sequencing device or computer processor.
61. A computer system for assessing tumor status of a subject with cancer, comprising:

a database that is configured to store (i) first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject, and (ii) second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; and

one or more computer processors operatively coupled to the database, wherein the one or more computer processors are individually or collectively programmed to:

determine, based on the first WGS data, (i) a first plurality of copy number aberrations (CNAs) in the first plurality of cfDNA molecules and (ii) a first plurality of fragment lengths of the first plurality of cfDNA molecules;

determine, based on the second WGS data, (iii) a second plurality of copy number aberrations (CNAs) in the second plurality of cfDNA molecules and (iv) a second plurality of fragment lengths of the second plurality of cfDNA molecules;

compare the first plurality of CNAs with the second plurality of CNAs to determine a CNA profile change;

determine a fragment length profile change based on the first plurality of fragment lengths and the second plurality of fragment lengths;

determine a first tumor fraction of the subject at the first timepoint or a second tumor fraction of the subject at the second timepoint, based at least in part on the CNA profile change and the fragment length profile change; and

detect a tumor status of the subject based at least in part on the first tumor fraction or the second tumor fraction.
62. A non-transitory computer-readable medium comprising machine-executable instructions which, upon execution by one or more computer processors, perform a method for assessing tumor status of a subject with cancer, the method comprising:

obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;

determining, based on the first WGS data, (i) a first plurality of copy number aberrations (CNAs) in the first plurality of cfDNA molecules and (ii) a first plurality of fragment lengths of the first plurality of cfDNA molecules;

obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;

determining, based on the second WGS data, (iii) a second plurality of copy number aberrations (CNAs) in the second plurality of cfDNA molecules and (iv) a second plurality of fragment lengths of the second plurality of cfDNA molecules;

comparing the first plurality of CNAs with the second plurality of CNAs to determine a CNA profile change;

determining a fragment length profile change based on the first plurality of fragment lengths and the second plurality of fragment lengths;

determining a first tumor fraction of the subject at the first timepoint or a second tumor fraction of the subject at the second timepoint, based at least in part on the CNA profile change and the fragment length profile change; and

detecting a tumor status of the subject based at least in part on the first tumor fraction or the second tumor fraction.
63. A method for assessing tumor status of a subject with cancer, comprising:

obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;

determining, based on the first MS data, an average methylation fraction for each of one or more CpG islands in the region of the genome, thereby obtaining a first average methylation fraction profile;

obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;

determining, based on the second MS data, an average methylation fraction for each of one or more CpG islands in the region of the genome, thereby obtaining a second average methylation fraction profile;

comparing the first average methylation fraction profile across the one or more CpG islands and the second average methylation fraction profile across the one or more CpG islands to determine a methylation fraction profile;

determining a first tumor fraction of the subject at the first timepoint or a second tumor fraction of the subject at the second timepoint, based at least in part on the respective methylation fraction profiles; and

detecting a tumor status of the subject based at least in part on the first tumor fraction or the second tumor fraction.
64. The method of embodiment 63, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.
65. The method of embodiment 63, wherein obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads.
66. The method of embodiment 65, wherein the methylation sequencing comprises whole genome bisulfite sequencing.
67. The method of embodiment 65, wherein the methylation sequencing comprises whole genome enzymatic methyl-seq.
68. The method of embodiment 65, wherein the methylation sequencing comprises oxidative bisulfite sequencing, TET-assisted pyridine borane sequencing (TAPS), TET-assisted bisulfite sequencing (TABS), oxidative bisulfite sequencing (oxBS-Seq), APOBEC-coupled epigenetic sequencing (ACE-seq), methylated DNA immunoprecipitation (MeDIP) sequencing, hydroxymethylated DNA immunoprecipitation (hMeDIP) sequencing, methylation array analysis, reduced representation bisulfite sequencing (RRBS-Seq), or cytosine 5-hydroxymethylation sequencing.
69. The method of embodiment 65, wherein the methylation sequencing is performed at a depth of no more than about 25×.
70. The method of embodiment 65, wherein the methylation sequencing is performed at a depth of no more than about 10×.
71. The method of embodiment 65, wherein the methylation sequencing is performed at a depth of no more than about 8×.
72. The method of embodiment 65, wherein the methylation sequencing is performed at a depth of no more than about 6×.
73. The method of embodiment 65, further comprising aligning the first or second plurality of sequencing reads to a reference genome, thereby producing a plurality of aligned sequencing reads.
74. The method of embodiment 65, further comprising enriching the first or second plurality of cfDNA molecules for the region of the genome.
75. The method of embodiment 74, wherein the enrichment comprises amplifying the first or second plurality of cfDNA molecules.
76. The method of embodiment 75, wherein the amplification comprises selective amplification.
77. The method of embodiment 75, wherein the amplification comprises universal amplification.
78. The method of embodiment 74, wherein the enrichment comprises selectively isolating at least a portion of the first or second plurality of cfDNA molecules.
79. The method of embodiment 78, wherein selectively isolating the at least the portion of the first or second plurality of cfDNA molecules comprises using a plurality of probes, each of the plurality of probes having sequence complementarity with at least a portion of the region of the genome.
80. The method of embodiment 78, wherein the at least the portion comprises a tumor marker locus.
81. The method of embodiment 80, wherein the at least the portion comprises a plurality of tumor marker loci.
82. The method of embodiment 81, wherein the plurality of tumor marker loci comprises one or more loci selected from The Cancer Genome Atlas (TCGA) or Catalogue of Somatic Mutations in cancer (COSMIC).
83. The method of embodiment 63, wherein the region of the genome comprises one or more of: CpG islands, CpG shores, patient-specific partially methylated domains, common partially methylated domains, promoters, gene bodies, evenly spaced genomewide bins, and transposable elements.
84. The method of embodiment 63, wherein the region of the genome comprises a plurality of non-overlapping regions of the genome.
85. The method of embodiment 84, wherein the plurality of non-overlapping regions of the genome have a pre-determined size.
86. The method of embodiment 85, wherein the pre-determined size is about 50 kilobases (kb), about 100 kb, about 200 kb, about 500 kb, about 1 megabases (Mb), about 2 Mb, about 5 Mb, or about 10 Mb.
87. The method of embodiment 84, wherein the plurality of non-overlapping regions of the genome comprises at least about 1,000 distinct regions.
88. The method of embodiment 87, wherein the plurality of non-overlapping regions of the genome comprises at least about 2,000 distinct regions.
89. The method of embodiment 63, wherein determining the first or second tumor fraction comprises comparing the methylation fraction profile with one or more reference methylation fraction profiles, wherein the one or more reference methylation fraction profiles are obtained from additional cfDNA molecules obtained or derived from additional bodily fluid samples of additional subjects.
90. The method of embodiment 89, wherein the additional subjects comprise one or more subjects with cancer.
91. The method of embodiment 89, wherein the additional subjects comprise one or more subjects without cancer.
92. The method of embodiment 89, wherein the additional subjects comprise one or more subjects having tumor progression.
93. The method of embodiment 89, wherein the additional subjects comprise one or more subjects not having tumor progression.
94. The method of embodiment 89, wherein the one or more reference methylation fraction profiles are obtained using additional bodily fluid samples of the subject obtained at one or more subsequent time points after the first timepoint.
95. The method of embodiment 63, further comprising detecting that the tumor status comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5.
96. The method of embodiment 63, further comprising detecting a major molecular response (MMR) of the subject when the first tumor fraction or the second tumor fraction is less than 0.01, less than 0.05, less than 0.1, less than 0.2, less than 0.3, less than 0.4, or less than 0.5.
97. The method of any one of embodiments 63-96, further comprising detecting the tumor status of the subject with a sensitivity of at least about 50%.
98. The method of embodiment 97, further comprising detecting the tumor status of the subject with a sensitivity of at least about 70%.
99. The method of embodiment 98, further comprising detecting the tumor status of the subject with a sensitivity of at least about 90%.
100. The method of any one of embodiments 63-99, further comprising detecting the tumor status of the subject with a specificity of at least about 50%.
101. The method of embodiment 100, further comprising detecting the tumor status of the subject with a specificity of at least about 70%.
102. The method of embodiment 101, further comprising detecting the tumor status of the subject with a specificity of at least about 90%.
103. The method of embodiment 102, further comprising detecting the tumor status of the subject with a specificity of at least about 98%.
104. The method of any one of embodiments 63-103, further comprising detecting the tumor status of the subject with a positive predictive value (PPV) of at least about 50%.
105. The method of embodiment 104, further comprising detecting the tumor status of the subject with a positive predictive value (PPV) of at least about 70%.
106. The method of embodiment 105, further comprising detecting the tumor status of the subject with a positive predictive value (PPV) of at least about 90%.
107. The method of any one of embodiments 63-106, further comprising detecting the tumor status of the subject with a negative predictive value (NPV) of at least about 50%.
108. The method of embodiment 107, further comprising detecting the tumor status of the subject with a negative predictive value (NPV) of at least about 70%.
109. The method of embodiment 108, further comprising detecting the tumor status of the subject with a negative predictive value (NPV) of at least about 90%.
110. The method of any one of embodiments 63-109, further comprising detecting the status progression of the subject with an area under the curve (AUC) of at least about 0.60.
111. The method of embodiment 110, further comprising detecting the tumor status of the subject with an area under the curve (AUC) of at least about 0.75.
112. The method of embodiment 111, further comprising detecting the tumor status of the subject with an area under the curve (AUC) of at least about 0.90.
113. The method of any one of embodiments 63-112, further comprising determining a tumor non-progression of the subject when tumor progression is not detected.
114. The method of any one of embodiments 63-113, further comprising, based on the determined tumor status of the subject, administering a therapeutically effective dose of a second therapeutic to treat the cancer of the subject.
115. The method of embodiment 114, wherein the second therapeutic comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor.
116. The method of any one of embodiments 63-115, wherein the first and the second pluralities of cfDNA molecules are from immune cells of the subject.
117. The method of any one of embodiments 63-116, wherein the detected tumor status is indicative of tumor progression, non-progression, regression, or recurrence.
118. The method of any one of embodiments 63-117, wherein the first and second MS data are obtained by a sequencing device or computer processor.
119. The method of any one of embodiments 1-60 and 63-118, wherein the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.
120. A computer system for assessing tumor status of a subject with cancer, comprising:

a database that is configured to store (i) first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject, and (ii) second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; and

one or more computer processors operatively coupled to the database, wherein the one or more computer processors are individually or collectively programmed to:

determine, based on the first MS data, an average methylation fraction for each of one or more CpG islands in the region of the genome, thereby obtaining a first average methylation fraction profile;

determine, based on the second MS data, an average methylation fraction for each of one or more CpG islands in the region of the genome, thereby obtaining a second average methylation fraction profile;

compare the first average methylation fraction profile across the one or more CpG islands and the second average methylation fraction profile across the one or more CpG islands to determine a methylation fraction profile;

determine a first tumor fraction of the subject at the first timepoint or a second tumor fraction of the subject at the second timepoint, based at least in part on the respective methylation fraction profiles; and

detect a tumor status of the subject based at least in part on the first tumor fraction or the second tumor fraction.
121. A non-transitory computer-readable medium comprising machine-executable instructions which, upon execution by one or more computer processors, perform a method for assessing tumor status of a subject with cancer, the method comprising:

obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;

determining, based on the first MS data, an average methylation fraction for each of one or more CpG islands in the region of the genome, thereby obtaining a first average methylation fraction profile;

obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;

determining, based on the second MS data, an average methylation fraction for each of one or more CpG islands in the region of the genome, thereby obtaining a second average methylation fraction profile;

comparing the first average methylation fraction profile across the one or more CpG islands and the second average methylation fraction profile across the one or more CpG islands to determine a methylation fraction profile;

determining a first tumor fraction of the subject at the first timepoint or a second tumor fraction of the subject at the second timepoint, based at least in part on the respective methylation fraction profiles; and

detecting a tumor status of the subject based at least in part on the first tumor fraction or the second tumor fraction.
122. The computer system of embodiment 120 or the non-transitory computer-readable medium of embodiment 121, wherein the detected tumor progression is based at least in part on one or more statistical modeling analyses of the respective methylation fraction profiles.
123. The system or medium of embodiment 122, wherein the one or more statistical modeling analyses comprise linear regression, simple regression, binary regression, Bayesian linear regression, Bayesian modeling, polynomial regression, Gaussian process regression, Gaussian modeling, binary regression, logistic regression, or nonlinear regression.
124. The system or medium of embodiment 122 or embodiment 123, wherein the one or more statistical modeling analyses compare the detected tumor progression with MS data derived from a sample having a known tumor fraction, MS data derived from a pure tumor sample, or MS data derived from a healthy sample.
125. A method for assessing tumor status of a subject with cancer, comprising:

obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;

determining, based on the first MS data, a methylation profile for each of one or more loci of the genome, thereby obtaining a first methylation profile;

obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;

determining, based on the second MS data, a methylation profile for each of one or more loci of the genome, thereby obtaining a second methylation profile;

comparing the first methylation profile across the one or more loci and the second methylation profile across the one or more loci;

determining a first tumor fraction of the subject at the first timepoint or a second tumor fraction of the subject at the second timepoint, based at least in part on the respective methylation profiles; and

detecting a tumor status of the subject based at least in part on the first tumor fraction or the second tumor fraction.
126. The method of embodiment 125, wherein the first and the second methylation profiles comprise 5-hydroxymethylcytosine status, 5-methylcytosine status, enrichment-based methylation assessment, median methylation level, mode methylation level, maximum methylation level, or minimum methylation level.
127. The method of embodiment 125 or embodiment 126, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.
128. The method of embodiment 125, wherein obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads.
129. The method of embodiment 128, wherein the methylation sequencing comprises whole genome bisulfite sequencing.
130. The method of embodiment 128, wherein the methylation sequencing comprises whole genome enzymatic methyl-seq.
131. The method of embodiment 128, wherein the methylation sequencing comprises oxidative bisulfite sequencing, TET-assisted pyridine borane sequencing (TAPS), TET-assisted bisulfite sequencing (TABS), oxidative bisulfite sequencing (oxBS-Seq), APOBEC-coupled epigenetic sequencing (ACE-seq), methylated DNA immunoprecipitation (MeDIP) sequencing, hydroxymethylated DNA immunoprecipitation (hMeDIP) sequencing, methylation array analysis, reduced representation bisulfite sequencing (RRBS-Seq), or cytosine 5-hydroxymethylation sequencing.
132. The method of embodiment 128, further comprising aligning the first or second plurality of sequencing reads to a reference genome, thereby producing a plurality of aligned sequencing reads.
133. The method of embodiment 128, further comprising enriching the first or second plurality of cfDNA molecules for the region of the genome.
134. The method of embodiment 128, wherein the region of the genome comprises one or more of: CpG islands, CpG shores, patient-specific partially methylated domains, common partially methylated domains, promoters, gene bodies, evenly spaced genomewide bins, and transposable elements.
135. The method of embodiment 128, wherein the region of the genome comprises a plurality of non-overlapping regions of the genome.
136. The method of embodiment 128, wherein determining the first or second tumor fraction comprises comparing the methylation fraction profile with one or more reference methylation fraction profiles, wherein the one or more reference methylation fraction profiles are obtained from additional cfDNA molecules obtained or derived from additional bodily fluid samples of additional subjects.
137. The method of embodiment 128, further comprising detecting that the tumor status comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5. 138. The method of embodiment 128, further comprising detecting a major molecular response (MMR) of the subject when the first tumor fraction or the second tumor fraction is less than 0.01, less than 0.05, less than 0.1, less than 0.2, less than 0.3, less than 0.4, or less than 0.5.
139. The method of any one of embodiments 128-138, further comprising determining a tumor non-progression of the subject when tumor progression is not detected.
140. The method of any one of embodiments 128-139, further comprising, based on the determined tumor status of the subject, administering a therapeutically effective dose of a second therapeutic to treat the cancer of the subject.
141. The method of any one of embodiments 128-140, wherein the first and the second pluralities of cfDNA molecules are from immune cells of the subject.
142. The method of any one of embodiments 128-141, wherein the detected tumor status is indicative of tumor progression, non-progression, regression, or recurrence.
143. The method of any one of embodiments 128-142, wherein the first and second MS data are obtained by a sequencing device or computer processor.
144. The method of any one of embodiments 128-143, wherein the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.
145. A computer system for assessing tumor status of a subject with cancer, comprising:

a database that is configured to store (i) first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject, and (ii) second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; and

one or more computer processors operatively coupled to the database, wherein the one or more computer processors are individually or collectively programmed to:

determine, based on the first MS data, a methylation profile for each of one or more CpG islands in the region of the genome, thereby obtaining a first methylation profile;

determine, based on the second MS data, a methylation profile for each of one or more CpG islands in the region of the genome, thereby obtaining a second methylation profile;

compare the first methylation profile across the one or more CpG islands and the second methylation profile across the one or more CpG islands;

determine a first tumor fraction of the subject at the first timepoint or a second tumor fraction of the subject at the second timepoint, based at least in part on the respective methylation profiles; and

detect a tumor status of the subject based at least in part on the first tumor fraction or the second tumor fraction.
146. A non-transitory computer-readable medium comprising machine-executable instructions which, upon execution by one or more computer processors, perform a method for assessing tumor status of a subject with cancer, the method comprising:

obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;

determining, based on the first MS data, a methylation profile for each of one or more CpG islands in the region of the genome, thereby obtaining a first methylation profile;

obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;

determining, based on the second MS data, a methylation profile for each of one or more CpG islands in the region of the genome, thereby obtaining a second methylation profile;

comparing the first average methylation fraction profile across the one or more CpG islands and the second average methylation fraction profile across the one or more CpG islands;

determining a first tumor fraction of the subject at the first timepoint or a second tumor fraction of the subject at the second timepoint, based at least in part on the respective methylation profiles; and

detecting a tumor status of the subject based at least in part on the first tumor fraction or the second tumor fraction.
147. A method for assessing tumor status of a subject with cancer, comprising:

obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;

determining, based on the first WGS data, (i) a first plurality of copy number aberrations (CNAs) in the first plurality of cfDNA molecules and (ii) a first plurality of fragment lengths of the first plurality of cfDNA molecules;

obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint;

determining, based on the first MS data, an average methylation fraction for each of one or more CpG islands in the region of the genome, thereby obtaining a first average methylation fraction profile;

obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;

determining, based on the second WGS data, (iii) a second plurality of copy number aberrations (CNAs) in the second plurality of cfDNA molecules and (iv) a second plurality of fragment lengths of the second plurality of cfDNA molecules;

obtaining second MS data of a second plurality of cell-free DNA (cfDNA) molecules across the region of the genome, wherein the second plurality of cfDNA molecules is obtained or derived from a bodily fluid sample of the subject at the second timepoint;

determining, based on the second MS data, an average methylation fraction for each of one or more CpG islands in the region of the genome, thereby obtaining a second average methylation fraction profile;

comparing the first plurality of CNAs with the second plurality of CNAs to determine a CNA profile change;

determining a fragment length profile change based on the first plurality of fragment lengths and the second plurality of fragment lengths;

comparing the first average methylation fraction profile across the one or more CpG islands and the second average methylation fraction profile across the one or more CpG islands to determine a methylation fraction profile;

determining a first tumor fraction of the subject at the first timepoint or a second tumor fraction of the subject at the second timepoint, based at least in part on the CNA profile change, the fragment length profile change, and the respective methylation fraction profiles; and

detecting a tumor status of the subject based at least in part on the first tumor fraction or the second tumor fraction.
148. A method for assessing tumor status of a subject with cancer, comprising:

obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;

determining, based on the first WGS data, (i) a first plurality of copy number aberrations (CNAs) in the first plurality of cfDNA molecules and (ii) a first plurality of fragment lengths of the first plurality of cfDNA molecules;

obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint;

determining, based on the first MS data, a methylation profile for each of one or more loci of the genome, thereby obtaining a first methylation profile;

obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;

determining, based on the second WGS data, (iii) a second plurality of copy number aberrations (CNAs) in the second plurality of cfDNA molecules and (iv) a second plurality of fragment lengths of the second plurality of cfDNA molecules;

obtaining second MS data of a second plurality of cell-free DNA (cfDNA) molecules across the region of the genome, wherein the second plurality of cfDNA molecules is obtained or derived from a bodily fluid sample of the subject at the second timepoint;

determining, based on the second MS data, a methylation profile for each of one or more loci of the genome, thereby obtaining a second methylation profile;

comparing the first plurality of CNAs with the second plurality of CNAs to determine a CNA profile change;

determining a fragment length profile change based on the first plurality of fragment lengths and the second plurality of fragment lengths;

comparing the first methylation profile across the one or more loci and the second methylation profile across the one or more loci;

determining a first tumor fraction of the subject at the first timepoint or a second tumor fraction of the subject at the second timepoint, based at least in part on the CNA profile change, the fragment length profile change, and the respective methylation fraction profiles; and

detecting a tumor status of the subject based at least in part on the first tumor fraction or the second tumor fraction.
149. The method of embodiment 148, wherein the first and the second methylation profiles comprise 5-hydroxymethylcytosine status, 5-methylcytosine status, enrichment-based methylation assessment, median methylation level, mode methylation level, maximum methylation level, or minimum methylation level.
150. The method of any one of embodiments 147-149, wherein the first WGS data and the first MS data are obtained from the same sample.
151. The method of any one of embodiments 147-149, wherein the first WGS data and the first MS data are obtained from different samples.
152. The method of any one of embodiments 147-151, wherein the second WGS data and the second MS data are obtained from the same sample.
153. The method of any one of embodiments 147-151, wherein the second WGS data and the second MS data are obtained from different samples.
154. The method of any one of embodiments 147-153, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.
155. The method of any one of embodiments 147-154, wherein obtaining the first WGS data comprises sequencing the first plurality of cfDNA molecules to generate a first plurality of sequencing reads, or wherein obtaining the second WGS data comprises sequencing the second plurality of cfDNA molecules to generate a second plurality of sequencing reads.
156. The method of any one of embodiments 147-155, further comprising enriching the first or second plurality of cfDNA molecules for a plurality of genomic regions.
157. The method of embodiment 155 or embodiment 156, wherein determining the first plurality of CNAs comprises determining quantitative measures of CNAs at each of at each of a plurality of genomic regions of the first plurality of sequencing reads, and wherein determining the second plurality of CNAs comprises determining quantitative measures of CNAs at each of at each of the plurality of genomic regions of the second plurality of sequencing reads.
158. The method of any one of embodiments 147-157, wherein determining the CNA profile change comprises comparing the first plurality of CNAs and the second plurality of CNAs with a plurality of reference CNA values, wherein the plurality of reference CNA values is obtained from additional cfDNA molecules obtained or derived from additional bodily fluid samples of additional subjects.
159. The method of any one of embodiments 147-158, wherein the first and second WGS data are obtained by pyrosequencing, sequencing-by-synthesis, single-molecule sequencing, Nanopore sequencing, semiconductor sequencing, sequencing-by-ligation, sequencing-by-hybridization, massively parallel sequencing, chain termination sequencing, single molecule real-time sequencing, Polony sequencing, combinatorial probe anchor synthesis, or hybrid capture-based sequencing.
160. The method of any one of embodiments 147-159, wherein obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads.
161. The method of any one of embodiments 147-160, further comprising enriching the first or second plurality of cfDNA molecules for the region of the genome.
162. The method of any one of embodiments 147-161, wherein the region of the genome comprises one or more of: CpG islands, CpG shores, patient-specific partially methylated domains, common partially methylated domains, promoters, gene bodies, evenly spaced genomewide bins, and transposable elements.
163. The method of any one of embodiments 147-162, wherein determining the first or second tumor fraction comprises comparing the methylation fraction profile with one or more reference methylation fraction profiles, wherein the one or more reference methylation fraction profiles are obtained from additional cfDNA molecules obtained or derived from additional bodily fluid samples of additional subjects.
164. The method of any one of embodiments 147-163, further comprising, based on the determined tumor status of the subject, administering a therapeutically effective dose of a treatment to treat the cancer of the subject.
165. The method of embodiment 164, wherein the treatment comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor.
166. The method of any one of embodiments 147-165, wherein the first and the second pluralities of cfDNA molecules are from immune cells of the subject.
167. The method of any one of embodiments 147-166, wherein the detected tumor status is indicative of tumor progression, non-progression, regression, or recurrence.
168. The method of any one of embodiments 147-167, wherein the first and second MS data are obtained by a sequencing device or computer processor.
169. The method of any one of embodiments 147-168, wherein the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.
170. The method of any one of embodiments 63-119, 125-144, and 147-169, wherein the region(s) of the genome comprises one or more MAGE (melanoma-associated antigen) genes, e.g., human MAGE genes.
171. The method of any one of embodiments 63-119, 125-144, and 147-169, wherein the region(s) of the genome comprises one or more promoters corresponding to one or more MAGE (melanoma-associated antigen) genes, e.g., human MAGE genes.

### EXAMPLES

### Example 1

### Early detection of Molecular Disease Progression by Whole-Genome Circulating Tumor DNA in Advanced Solid Tumors

Treatment response assessment for patients with advanced solid tumors may be complex, and other methods of assessment may require greater precision for early disease assessment. Current guidelines may rely on imaging, which may impose limitations such as a long time duration needed before treatment effectiveness can be determined. Using methods and systems of the present disclosure, serial changes in whole-genome (WG) circulating tumor DNA (ctDNA) were used to detect disease progression early in the treatment course.

A set of 97 patients with advanced cancer were enrolled, and blood samples were collected from each of the patients before and after initiation of a new treatment. Plasma cell-free DNA libraries were prepared for either WG or WG bisulfite sequencing. Longitudinal changes in the fraction of ctDNA were quantified to identify molecular progression or response in a binary manner (e.g., “progression” or “non-progression”). Study endpoints were agreement with first follow-up imaging (FUI) and stratification of progression-free survival (PFS).

The results demonstrated that patients with early molecular progression had shorter progression-free survival (PFS) (n=14; median=62 days) compared to other patients without early molecular progression (n=78; median=263 days, hazard ratio (HR)=12.6 [95% confidence interval (CI) of 5.8 to 27.3], log-rank P<1E-10, 5 excluded from analysis). All cases with molecular progression were confirmed by FUT, and molecular progression preceded FUI by a median of 40 days. Clinical progression was identified in the patients with a sensitivity of 54% and a specificity of 100%, at a median of 24 days into treatment.

Molecular progression, identified based on ctDNA data, successfully detected disease progression in cancer patients for cases on treatment with high specificity approximately 6 weeks before follow-up imaging. This approach may enable early course change to a potentially effective therapy, thereby avoiding side effects and cost associated with cycles of ineffective treatment.

The methods and systems of the present disclosure may have significant translational relevance. Tools for early assessment of treatment response in advanced solid tumors may require refinement. Using methods and systems of the present disclosure, baseline and early serial assessments of WG ctDNA were performed to predict treatment response prior to standard-of-care clinical and radiographic assessments. The results demonstrated that the blood-based prediction approach reliably identified molecular progression, approximately 6 weeks before imaging, with very high specificity and positive predictive value across multiple different tumor types and treatment types. Patients with molecular progression had significantly shorter progression-free survival (PFS) compared with non-progressor patients. In addition, a large quantitative decrease in tumor fraction ratio was associated with significant durable benefit. Collectively, these results demonstrate that cancer-related changes in the blood precede clinical or imaging changes and may inform changes in clinical management earlier in the treatment course to improve long-term patient outcomes and limit cost.

The current standard of care for evaluation of treatment response for advanced solid tumors in cancer patients may be based on physical exam of the patient, patient-reported symptoms, and periodic radiographic tumor assessment of the patient. However, there may be limitations to these approaches, since subtle changes in disease are often asymptomatic, and there are considerable costs, uncertainty, and patient anxiety associated with frequent imaging. In clinical trials, response criteria are standardized (e.g., RECIST, irRECIST) to guide evaluation by comparing a baseline scan before treatment initiation with periodic follow-up imaging (FUI) with pre-specified criteria for response. These criteria can be limited by the reliability of measurements over time, difficulty of measuring sites of disease (e.g., bone or pleural effusions), and challenges with distinguishing pseudo-progression (e.g., false positive cases of progression) from true progression (e.g., true positive cases). Therefore, improved methods for monitoring response to treatment may be advantageous, given the emergence of new treatment modalities with ongoing questions regarding how best to manage clinical treatment, minimize toxicity to the patient, and control costs. Here, a blood-based approach using dynamic changes in WG ctDNA was evaluated for early assessment of treatment response.

Liquid biopsy assays may analyze circulating cell-free DNA (cfDNA), circulating tumor cells (CTCs), ribonucleic acid (RNA), proteins, exosomes, microbiome, or metabolites. ctDNA may likely originate from cancer cells undergoing apoptosis, necrosis, or potential active mechanisms involving nucleic acid secretion to facilitate metastasis and gene expression at distant sites. The amount of ctDNA may correlate with tumor burden and/or more advanced stages of disease, and may also be affected by tumor type, origin, location of metastasis, and treatment. There may be several distinguishing features between ctDNA and non-tumor cfDNA. Specifically, as compared with cfDNA, ctDNA may contain one or more of: tumor-specific somatic point mutations, structural variations, shorter fragment lengths, biased fragment start and end positions, and changes in epigenetic patterns. Copy number aberrations (CNAs), which may include deletions, duplications, or higher copy amplifications of portions of the genome, may be a common form of structural variation that us observed in patients with advanced disease at various sites across the genome. Further, CNAs may be shown to be detectable in cfDNA from patients by low-pass next-generation sequencing (NGS), with CNAs being detected at a higher rate in patients with advanced disease. However, changes in CNAs over time in advanced cancer patients may remain understudied.

Recently, there has been significant interest in evaluating the potential clinical utility of ctDNA in advanced solid tumors. For example, the prognostic value of ctDNA genomic alterations and mutant allele frequency as surrogates for tumor burden may be demonstrated. In the adjuvant setting, residual ctDNA after surgery (e.g., indicative of minimal residual disease) may be associated with disease recurrence across multiple different tumor types. In advanced disease, a well-validated clinical use may be to identify driver mutations with known drug targets. Additionally, resistance mutations may be identified in the blood, guiding clinical management. The potential role of ctDNA for tumor response assessment may be evaluated in tumor types such as melanoma, non-small cell lung cancer, breast cancer, and prostate cancer; however, the clinical utility for routine assessment may not be established yet.

Here, in a prospectively enrolled, advanced stage, pan-cancer cohort, whole-genome cfDNA analysis was performed as a molecular marker or indicator of disease progression earlier in the treatment course, as compared with routine clinical and radiographic assessment of disease. In contrast to analyzing particular genes, this approach utilized CNAs and fragmentation patterns across the genome, a technique with broad potential clinical applications across multiple different tumor types. Furthermore, for a subset of patients, bisulfite conversion was performed as part of the assay, which provided insight into genome-wide methylation changes. It was hypothesized that early changes in cancer-associated signals in the blood would be predictive of response status at the time of first FUI and that the magnitude of the dynamic change in signal would provide longer-term prognostic information across a variety of solid tumor types and treatments.

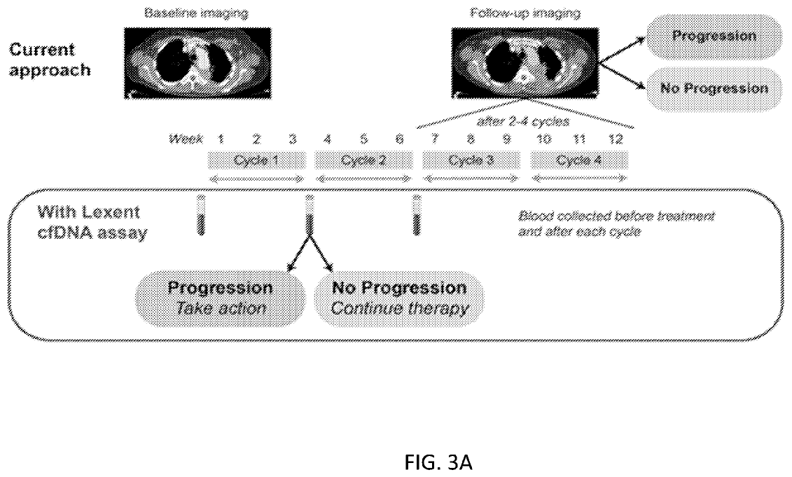

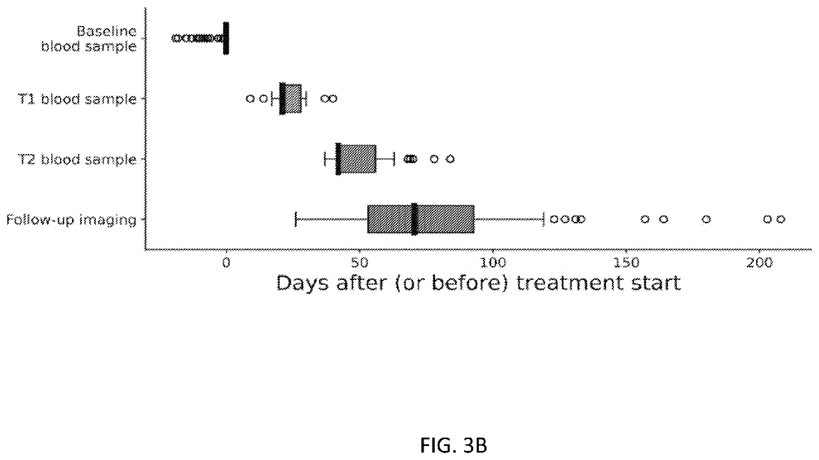

Study samples of subjects and patients were obtained as follows. The study sample here represented a subset of a currently accruing longitudinal observational study. From May 2017 to December 2018, participants (age over 18 years) were prospectively enrolled from five oncology centers in the United States (TMPN—Cancer Care, Redondo Beach, Calif.; Scripps≥California Cancer Associates, San Diego, CA; Sharp Memorial Hospital, San Diego, Calif.; Summit Cancer Centers, Post Falls, Id.; Robert H. Lurie Comprehensive Cancer Center of Northwestern University, Chicago, IL) and followed through June 2019 (as shown in Table 1). Eligibility criteria included the following: diagnosis of a non-hematologic and surgically unresectable advanced tumor (stage III or higher) at presentation; commencement of a new systemic treatment regimen of the physician's choice; and presence of either measurable or evaluable disease by imaging. To be included in this cohort, the participants needed to have venous blood samples from at least two time points: a baseline (at time=T0, before treatment initiation) and another one before cycle 2 (at time=T1) and/or cycle 3 (at time=T2, as shown in FIG. 3A). This study was conducted in accordance with the Declaration of Helsinki and approved by Northwestern University, Sharp Memorial Hospital, and Western Institutional Review Boards. An informed consent was obtained from each patient prior to participation in the study.

TABLE 1
Participant characteristics. Distribution of blood
sample and follow-up times are with respect to
on-study treatment times, May 2017-June 2019

| | Median (Min-Max) | | N = 92 (%) | |
|---|---|---|---|---|
| Age | 70 | (30-89) | | |
| Sex | | | | |
| Female | | | 51 | (55.4) |
| Male | | | 41 | (44.6) |
| Cancer type | | | | |
| Lung | | | 40 | (43.5) |
| Breast | | | 25 | (27.2) |
| GI | | | 14 | (15.2) |
| GU | | | 6 | (6.5) |
| Melanoma | | | 6 | (6.5) |
| Sarcoma | | | 1 | (1.1) |
| Treatment types | | | | |
| Chemotherapy | | | 32 | (34.8) |
| Chemotherapy, Antibody | | | 10 | (10.9) |
| Immunotherapy | | | 25 | (27.2) |
| Immunotherapy, Chemotherapy | | | 9 | (9.8) |
| Endocrine | | | 3 | (3.3) |
| Endocrine, CDK4/6i | | | 7 | (7.6) |
| Targeted alone | | | 6 | (6.5) |
| Lines of therapy | | | | |
| 1 | | | 48 | (52.2) |
| 2 | | | 23 | (25.0) |
| 3+ | | | 21 | (22.8) |
| T1 (days) | 21 | (9-40) | 86 | (93.5) |
| T2 (days)* | 42 | (37-84) | 66 | (71.7) |
| First follow-up (days) | 71 | (26-208) | | |
| Last follow-up (days) | 140 | (35-645) | | |

*60 participants had both post-treatment time points

FIGS. 3A-3B show an overview of the clinical setting, in accordance with some embodiments. FIG. 3A shows a diagram comparing radiographic response assessment and the potential use of cfDNA to assess molecular response. FIG. 3B shows timing of imaging and blood collections for patients in the study.

Evaluation of response status was performed as follows. Participants were radiologically assessed at baseline and again at first follow-up, as determined per standard-of-care routine clinical assessment. The primary endpoint of the study was evidence of radiographic progression (e.g., as determined by Response Evaluation Criteria in Solid Tumors (RECIST) version 1.1) or clinical response evaluation. Measurable disease by imaging was interpreted by the treating physician facility and an independent radiologist, who was blinded to the assessment of molecular response. When RECIST outcome could not be ascertained due to either non-evaluable or missing imaging study, the clinical response evaluation was used. Clinical response was defined as the physician's outcome assessment before a treatment change, and was categorized as clinical progressive disease (PD), responsive disease (non-PD), stable disease (non-PD), or too early to assess.

PFS was defined as the time from the start of treatment to first documentation of PD, or death due to any cause, whichever occurred first. Patients last known to be alive and progression-free were censored at the date of last contact. Patients were considered as lost-to-follow-up if they were no longer part of the study and their status was unknown (non-assessable case).

Sample preparation was performed as follows. At each time point, 10 mL of whole blood was collected in Streck Cell-Free DNA blood collection tubes (BCT). Plasma was separated via centrifugation at 1600×g for 15 minutes, followed by centrifugation at 2500×g for 10 minutes within 7 days from the time of collection. cfDNA was extracted from plasma using the Qiagen QIAmp MinElute ccfDNA kit and stored at Δ80° C. until library preparation. For each patient, libraries were prepared using the KAPA HyperPrep library prep kit for whole genome sequencing (WGS) (n=54 patients) or the Nugen Ovation Ultralow Methyl-Seq whole genome bisulfite sequencing kit (WGBS) (n=43 patients). Average input cfDNA into the library preparation was 20 nanograms (ng). Libraries were sequenced on an Illumina HiSeq X platform to an average depth of 20× (with a range of 6× to 29×).

Bioinformatics methods were performed as follows. Tumor fraction ratio (TFR) was measured to assess changes in ctDNA using CNAs and local changes in cfDNA fragment length, both assessed from sequencing data. Reads were aligned to the human genome (GRCh37) with a custom bioinformatics pipeline based on BWA, sambamba, and samtools. Reads were then de-duplicated, and GC biases were corrected using the deepTools software package. CNAs were detected using a pipeline based on ichorCNA and custom algorithms. Normalized fragment length was computed by normalizing for the median of the fragment length distribution in the library and genomic location across multiple unaffected libraries. Background signals for CNA and fragment length were established for each sequencing protocol using healthy normal samples taken from 44 individuals with no current or prior diagnosis of any malignancy (as shown in Table S1). In order to maximize sensitivity of CNAs while preventing false positive detections, Spearman's rank correlation between local mean fragment length and copy number was used as a disqualifier of CNA call sets.

TABLE S1
Longitudinal samples from a healthy participant cohort, including
44 healthy participants processed with both WGS and WGBS

| | Median (Min-Max) | N = 44 (%) |
|---|---|---|
| Age | 64 (27-82) | |
| Sex | | |
| Female | | 29 (66) |
| Male | | 15 (34) |
| Days between | 37 (14-180) | |
| longitudinal samples | | |
| WGS | | 27 (61) |
| Number of timepoints | | |
| per participant | | |
| 2 | | 27 (61) |
| WGBS | | 21 (48) |
| Number of timepoints | | |
| per participant | | |
| 2 | | 15 (34) |
| 3 | | 6 (14) |

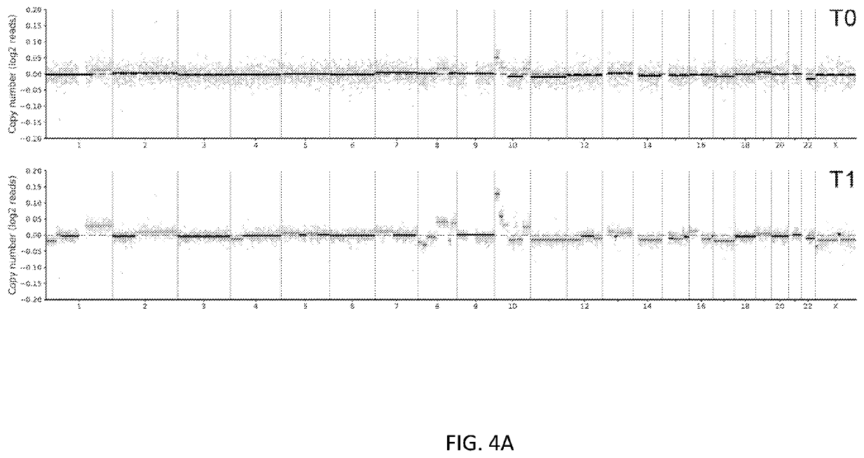

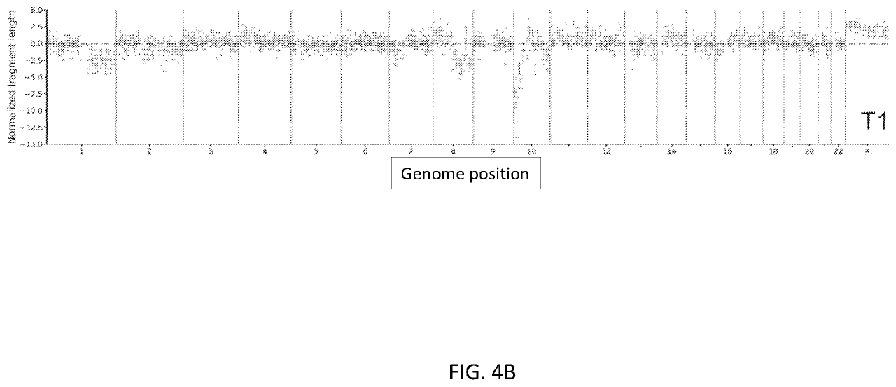

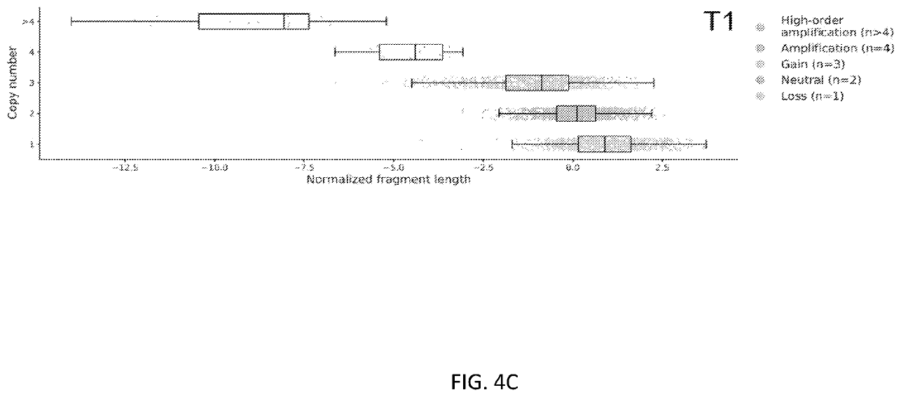

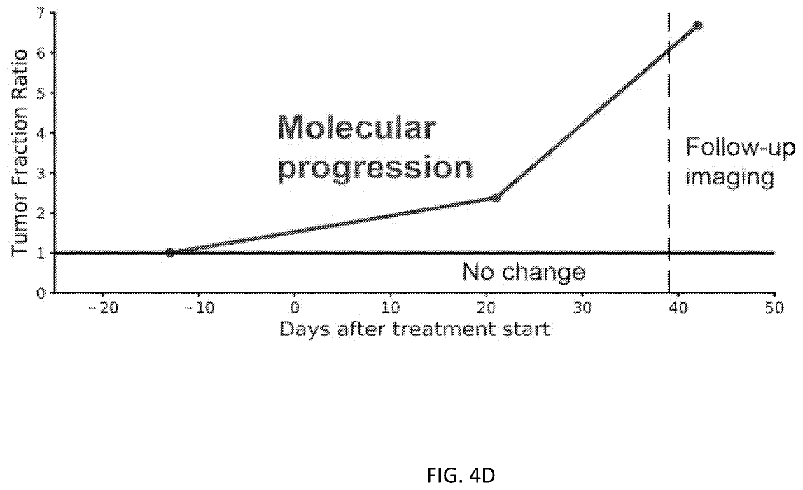

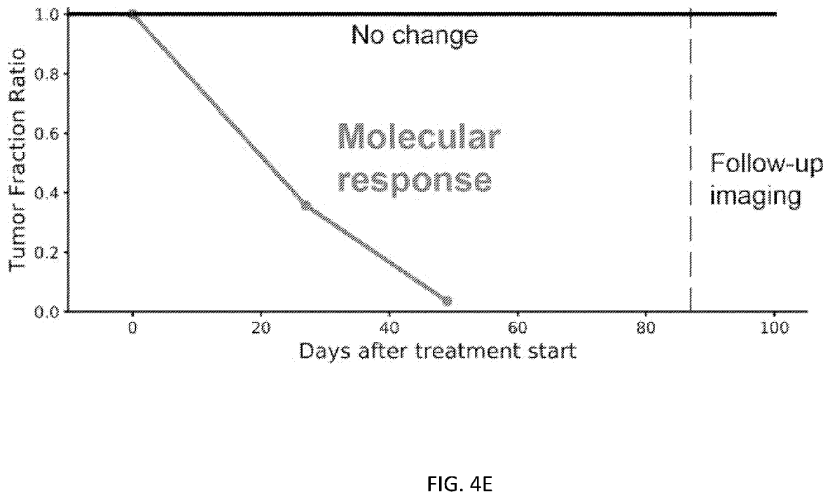

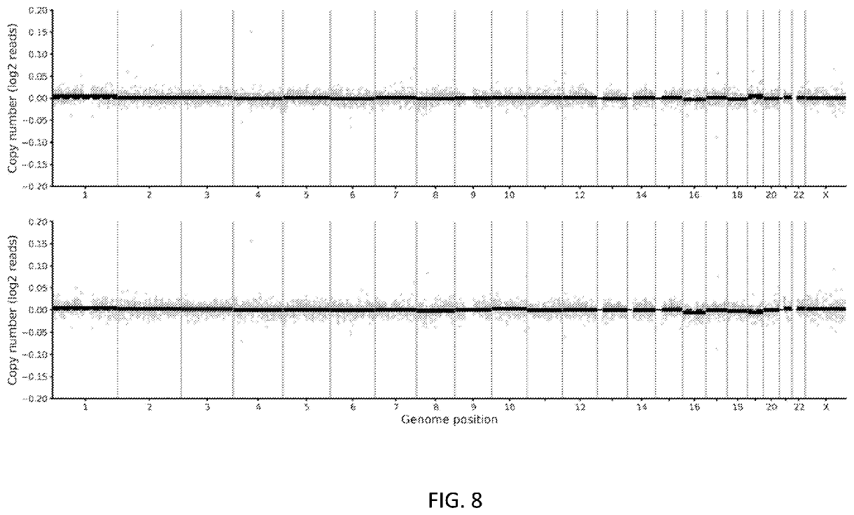

Assessing changes in ctDNA over time via a direct comparison of the CNA-derived estimate of TF between a patient's time points may not always be reliable because there may be ambiguity in which read depth levels correspond to which structural events. For example, two regions may be called as either a neutral region and a duplicated region, or a heterozygous deletion and a neutral region, in a highly mutated tumor where there is an ambiguous neutral level. To circumvent this challenge, CNAs detected at multiple time points were compared longitudinally with a linear model to quantify TFR. In order to determine confident calls, measured changes were compared to a simulated background model and required to exceed a Z-score threshold of 3. No longitudinal comparisons of samples from 44 healthy participants (Table S1) showed a significant change in TFR. For example, FIG. 8 shows longitudinal WGS data for a healthy individual, in accordance with some embodiments. This figure includes genome-wide plots showing no CNAs detected for participant LB-S00129 at an initial blood draw (top) and 34 days later (bottom), as in FIG. 4A. Cases that showed a confident increase in tumor fraction (e.g., as indicated by TFR greater than 1) at either time point were classified as molecular progression. Major molecular response (MMR) was defined as TFR<0.1.

Statistical analysis was performed as follows. For the purposes of calculating sensitivity, specificity, positive predictive value, and negative predictive value, true positives were defined as cases where the assay showed a molecular progression which were also evaluated as PD either clinically or by RECIST 1.1 at first FUI; true negatives were cases where both the assay and clinical evaluation did not call PD at first follow-up. False positives and false negatives were defined as cases where molecular response assessment disagreed with the first FUI with either a clinical or radiographic progression or lack of a clinical or radiographic progression respectively. Confidence intervals on these sensitivity, specificity, positive predictive value, and negative predictive value metrics were computed with the Wilson's score interval method.

Survival curves were generated using the Kaplan-Meier method, and differences in PFS between molecular progressors and non-progressors assessed using the log-rank test. The Cox proportional hazards model was used for the assessment of the effects of different magnitudes of change on PFS. Statistical analyses of survival were performed with the R survival package version 2.41-3, and other statistical analyses were determined using the python scipy package version 1.1.0.

Patient characteristics were obtained as follows. A total of 97 patients with advanced cancer, who fit the inclusion criteria of the study and had appropriate long-term follow-up data, had samples obtained and sequenced for the analysis. Baseline blood samples failed sequencing for five participants, therefore they were excluded. The remaining 92 patients with NGS and clinical outcome data were included in this analysis. The median age was 70 years and 55% were females (as shown in Table 1). About half of the participants received a first-line therapy (52%) and 25% received a second-line therapy. The majority of patients had lung cancer (44%) or breast cancer (27%) with the remainder representing gastrointestinal cancers (15%), genitourinary cancers (6.5%), melanoma (6.5%), and sarcoma (1%). During the study, 46% of all participants received chemotherapy (with or without antibody treatment) and 37% received immunotherapy with or without chemotherapy (n=34, Table 1, Table S2). The first FUI occurred a median of 71 days after treatment start (FIG. 3B, range of 26 to 208 days). Three participants had a non-evaluable imaging study and were assessed as clinical non-PD at week 12; and two participants did not have an imaging study prior to treatment change and were assessed as clinical PD at week-9 and week-19 by the treating physician. The median follow-up time of the full cohort was 140 days (range of 35 to 645 days).

TABLE S2
List of on-study therapies, including summary
of participants by drug name and drug class

| | | Participant |
|---|---|---|
| Drug Names | Drug Class | Count |
| abiraterone acetate | Endocrine | 1 |
| anastrozole | Endocrine | 1 |
| atezolizumab | Immunotherapy | 1 |
| atezolizumab, entinostat | Immunotherapy, HDACi | 1 |
| capecitabine | Chemotherapy | 3 |
| capecitabine, bevacizumab | Chemotherapy, Antibody | 1 |
| capecitabine, trastuzumab, zoledronic acid, | Chemotherapy, Antibody | 1 |
| tucatinib | | |
| carboplatin, docetaxel, pertuzumab, | Chemotherapy, Antibody | 1 |
| trastuzumab, zoledronic acid | | |
| carboplatin, etoposide | Chemotherapy | 1 |
| carboplatin, gemcitabine | Chemotherapy | 3 |
| carboplatin, nab-paclitaxel | Chemotherapy | 1 |
| carboplatin, nab-paclitaxel, pembrolizumab | Immunotherapy, | 2 |
| | Chemotherapy | |
| carboplatin, paclitaxel | Chemotherapy | 4 |
| carboplatin, paclitaxel, pembrolizumab | Immunotherapy, | 1 |
| | Chemotherapy | |
| carboplatin, pembrolizumab, pemetrexed | Immunotherapy, | 5 |
| | Chemotherapy | |
| carboplatin, pembrolizumab, pemetrexed, | Immunotherapy, | 1 |
| zoledronic acid | Chemotherapy | |
| carboplatin, pemetrexed | Chemotherapy | 2 |
| carboplatin, pemetrexed, bevacizumab | Chemotherapy, Antibody | 1 |
| cisplatin, etoposide | Chemotherapy | 4 |
| cyclophosphamide, methotrexate, trastuzumab | Chemotherapy, Antibody | 1 |
| docetaxel, pertuzumab, trastuzumab | Chemotherapy, Antibody | 2 |
| docetaxel, zoledronic acid | Chemotherapy | 1 |
| eribulin | Chemotherapy | 1 |
| erlotinib | Targeted | 1 |
| fulvestrant, denosumab | Endocrine, CDK4/6i | 1 |
| fulvestrant, palbociclib | Endocrine, CDK4/6i | 2 |
| fulvestrant, palbociclib, zoledronic acid | Endocrine, CDK4/6i | 1 |
| gemcitabine, cisplatin | Chemotherapy | 1 |
| gemcitabine, nab-paclitaxel | Chemotherapy | 4 |
| Ipilimumab, nivolumab | Immunotherapy | 1 |
| irinotecan, 5-FU | Chemotherapy | 1 |
| lapatinib, trastuzumab | Targeted | 1 |
| letrozole | Endocrine | 1 |
| anastrozole, leuprolide acetate, palbociclib | Endocrine, CDK4/6i | 1 |
| letrozole, palbociclib | Endocrine, CDK4/6i | 1 |
| letrozole, ribociclib | Endocrine, CDK4/6i | 1 |
| liposomal doxorubicin | Chemotherapy | 2 |
| liposomal doxorubicin, zoledronic acid | Chemotherapy | 1 |
| nab-paclitaxel | Chemotherapy | 2 |
| neratinib | Targeted | 1 |
| nivolumab | Immunotherapy | 11 |
| olaratumab, doxorubicin | Chemotherapy, Antibody | 1 |
| oxaliplatin, 5-FU, panitumumab | Chemotherapy, Antibody | 1 |
| pazopanib | Targeted | 2 |
| pembrolizumab | Immunotherapy | 9 |
| pembrolizumab, zoledronic acid | Immunotherapy | 1 |
| pemetrexed | Chemotherapy | 1 |
| ramucirumab, paclitaxel | Chemotherapy, Antibody | 1 |
| regorafenib | Targeted | 1 |
| nivolumab, zoledronic acid | Immunotherapy | 1 |
| Grand Total | | 92 |

Baseline blood samples were collected prior to treatment start for all patients (FIG. 3B, median=0 days after treatment start, minimum=19 days before treatment start). One or two post-treatment samples were collected for each patient, with sample T1 collected prior to the second cycle of therapy at a median of 21 days after treatment start (n=86, range of 9 to 40 days) and sample T2 collected prior to the third cycle of therapy at a median of 42 days (n=66, range of 37 to 84 days). Both post-treatment samples were collected for 60 patients.

FIGS. 4A-4E show serial assessment of ctDNA to determine molecular progression, in accordance with some embodiments. FIG. 4A shows the genome-wide plots of CNAs detected for patient LS030178. The TO baseline blood draw was collected 13 days before the start of treatment, and T1 was collected 21 days after the start of treatment. FIG. 4B shows that normalized fragment length exhibit the reverse pattern compared to CNAs. FIG. 4C shows that overall there was a strong negative correlation between the normalized fragment length at each genomic position and the inferred copy number (Spearman's rho=-0.57, P<1E-10). FIG. 4D shows that patient LS030178 had an increase in TFR at follow-up time points T1 and T2, detectable in advance of imaging that indicated progressive disease. FIG. 4E shows that patient LS030093, who responded to therapy, showed a marked decrease in TFR at T1 and T2, concordant with later imaging that showed a partial response.

Serial measurements of ctDNA showed rapid changes early on treatment. Changes in tumor fraction were assessed using WG analysis to quantify the TFR between baseline and post-treatment samples. Substantial changes in TFR were observed early after treatment initiation (FIGS. 4A-4D). Patient LS030178 demonstrated an example of a rapid increase in TFR to 2.4 at time T1 following the first cycle of therapy, indicating a major increase from baseline, followed by an even greater increase at time T2 (FIGS. 4A-4D). This patient had a somatic gain of the long arm of chromosome 1 (1q), which may be one of the most common arm-level aberrations in breast cancer. Additionally, the strong pattern of CNAs was corroborated by the fragmentation pattern (FIGS. 4B-4C). Conversely, patient LS030093 showed a decrease in TFR at time T1 and then a larger decrease at time T2 (FIG. 4E).

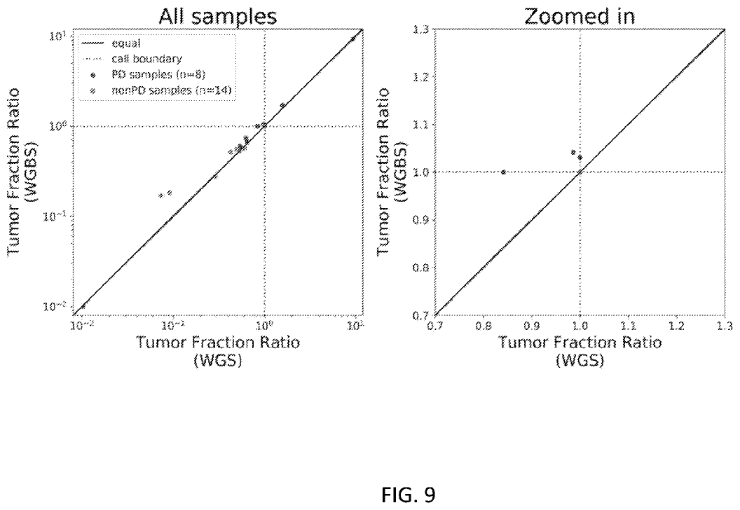

A subset of samples had both WGS and WGBS, and these were used to test whether TFR could be quantified equivalently across sequencing protocols. FIG. 9 shows a comparison of tumor fraction ratio across sequencing protocols, in accordance with some embodiments. This figure shows results for 20 post-treatment samples from 13 participants that were processed with both WGS and WGBS. Two samples from patients with PD at first FUI had discordant classifications of molecular progression, with measurements of TFR that were close to the call boundary. TFR values were highly concordant between WGS and WGBS, enabling analysis of the full cohort including samples analyzed with both protocols.

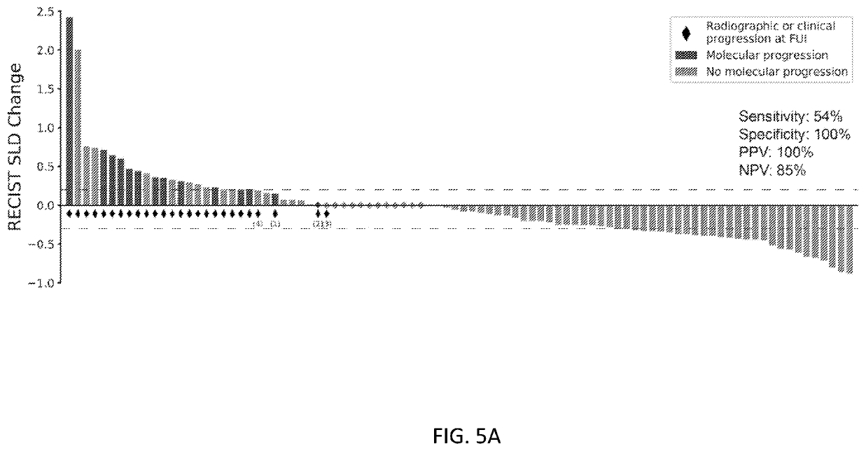

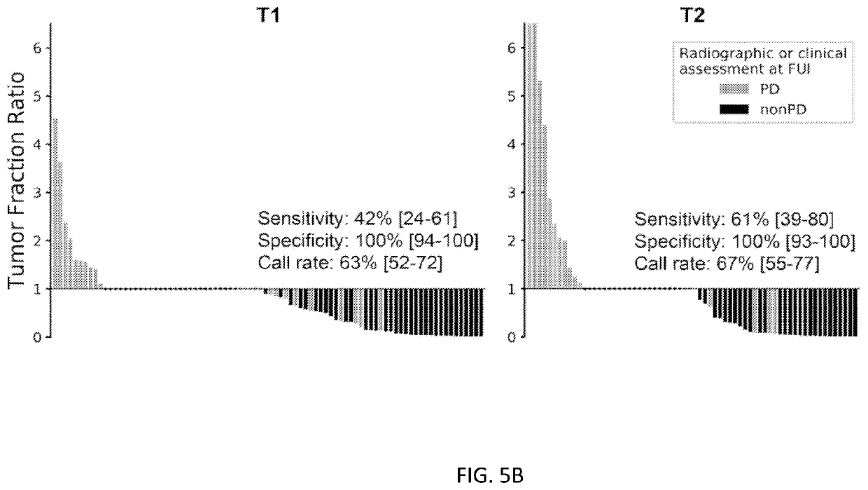

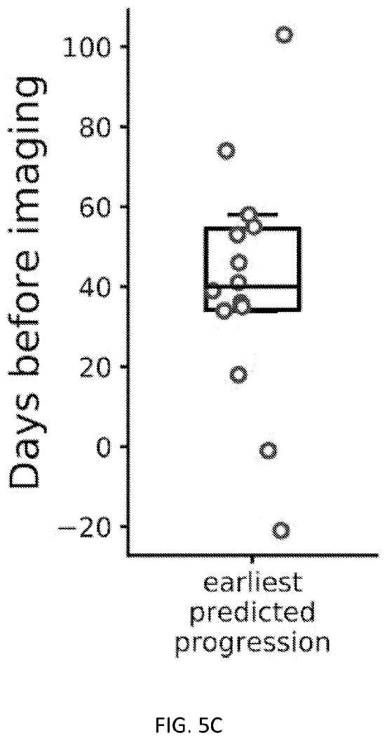

FIGS. 5A-5C show ctDNA assessments following first or second cycle of therapy predicted progression, in accordance with some embodiments. FIG. 5A shows a comparison of imaging results at first FUI (SLD assessed by RECIST 1.1) with ctDNA assessment of molecular progression, indicated by a confident increase in TFR for either post-treatment sample (sensitivity=54%, specificity=100%, PPV=100%, NPV=85%). Footnoted cases showed clear clinical progression. FIG. 5B shows TFR for progressors and non-progressors at T1 (left) and T2 (right), compared to radiographic or clinical assessment of PD or non-PD, showing predictive performance at each time point. FIG. 5C shows that for patients with molecular progression, detection of the molecular progression preceded the date of detection of progression by standard of care imaging by a median of 40 days (range of −21 to 103 days).

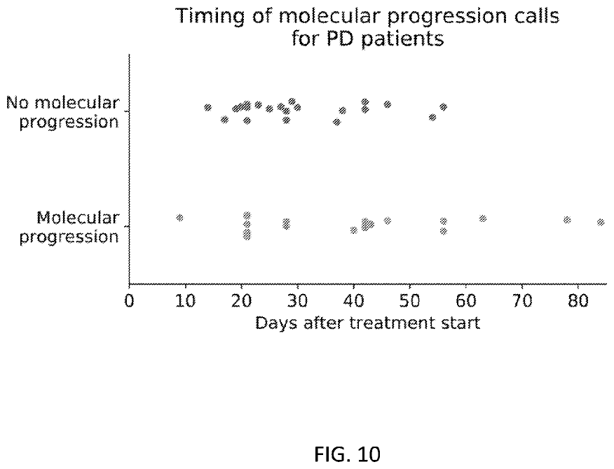

Tracking changes in tumor fraction at early time points predicted progression at first follow-up response assessment. At the first FUI and clinical evaluation, 26 of 92 patients had clinical PD and the 66 patients had non-PD. To evaluate the predictive value of the ctDNA assay, we compared the classification of molecular progression to clinical assessment at first FUI for all patients (FIG. 5A). All 14 patients with a molecular progression at either T1 or T2 had PD, and likewise of the 66 patients who were non-PD, none had molecular progression. The sensitivity of the assay, including time points T1 and T2, was 54% and the specificity was 100%. The sensitivity was 42% at T1 and was 61% at T2 (FIG. 5B). There was no statistically significant relationship between sensitivity and timing of the blood draw. FIG. 10 shows sample timing and sensitivity, in accordance with some embodiments. This figure shows molecular progression and blood sample timing for the 42 samples from 26 participants with PD at first FUI (two-sample Kolmogorov-Smirnov test, P=0.15). In the cases where molecular progression was called, the time point where it was first identified preceded the detection of progression by imaging by a median of 40 days (FIG. 5C).

Among the 12 patients with PD at first FUI where there was a discrepancy between imaging and molecular assessment of progression, 3 patients had no confident CNAs detected, 2 patients had no significant change in tumor fraction, and 7 patients had a decrease in tumor fraction. For the discordant cases with a decrease in tumor fraction, there were a variety of cancer types represented (3 breast cancer, 2 lung cancer, 1 renal cancer, 1 sarcoma cancer), treatments (4 chemotherapy, 1 immunotherapy, 1 endocrine therapy, 1 targeted therapy alone), and lines of therapy (3 first-line, 2 second-line, 1 third-line, and 1 fifth-line). Additionally, there were no major difference in predictive performance between WGS and WGBS (Table S3).

TABLE S3
Molecular progression and assessment
at first FUI by sequencing protocol

ctDNA results

| | Assessment | Molecular | No molecular |
|---|---|---|---|
| Patient subset | at FUI | Progression | progression |
| All patients | PD | 14 | 12 |
| | nonPD | 0 | 66 |
| WGS | PD | 11 | 11 |
| | nonPD | 0 | 32 |
| WGBS | PD | 3 | 1 |
| | nonPD | 0 | 34 |

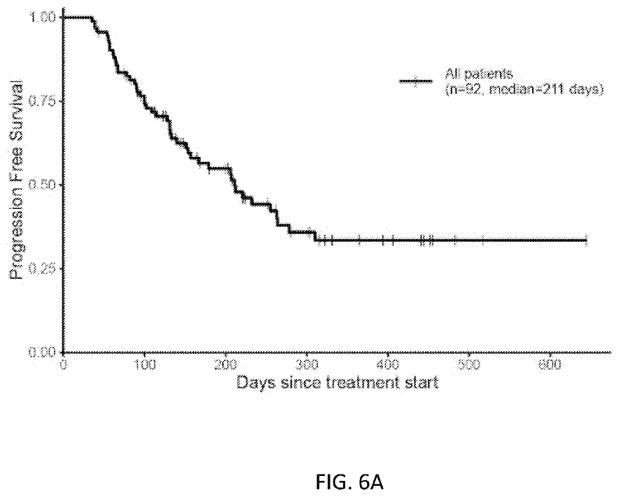

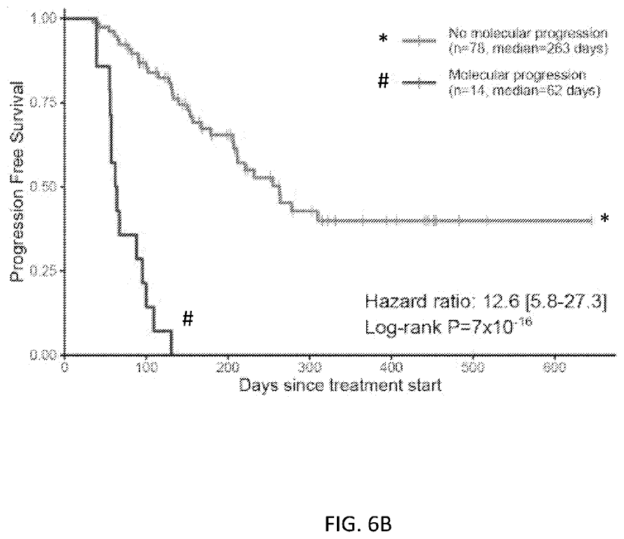

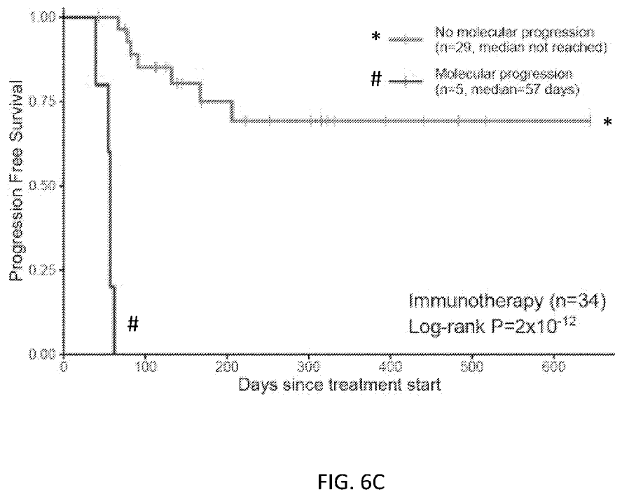

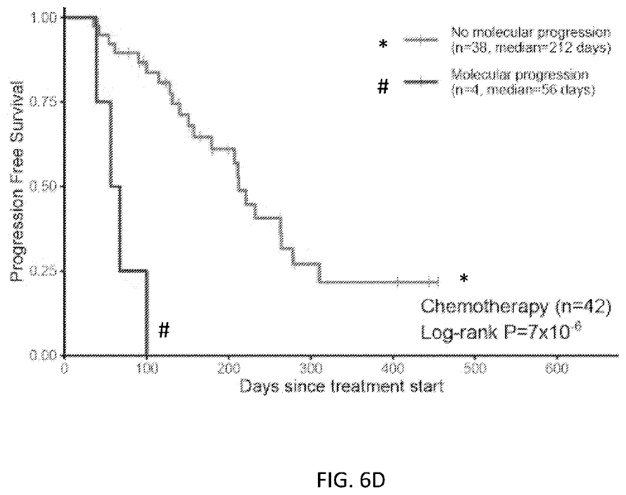

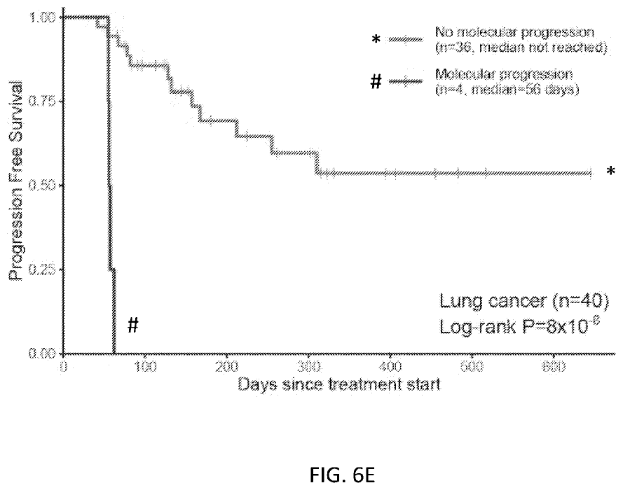

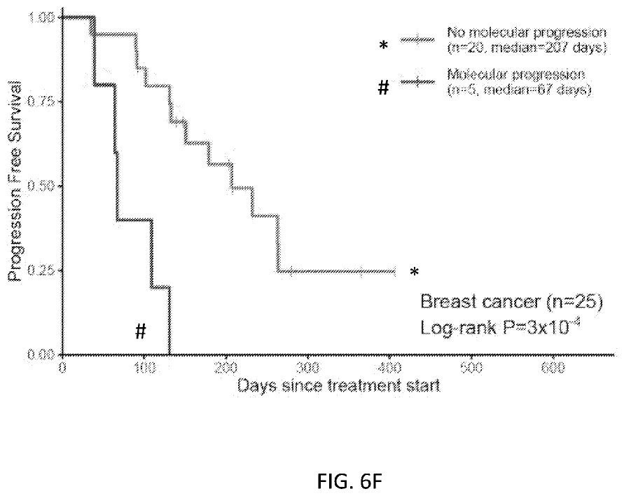

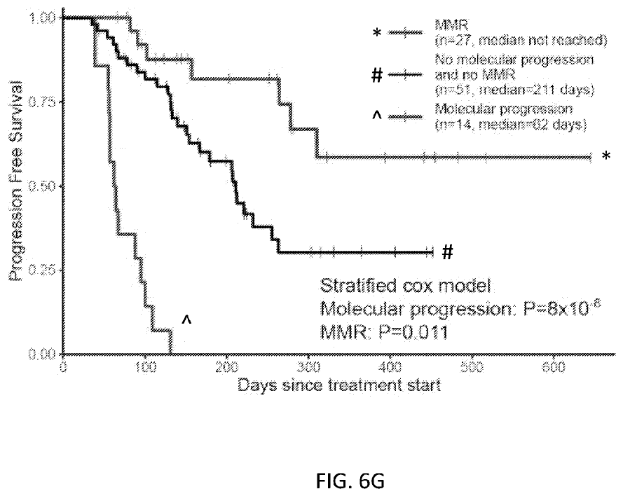

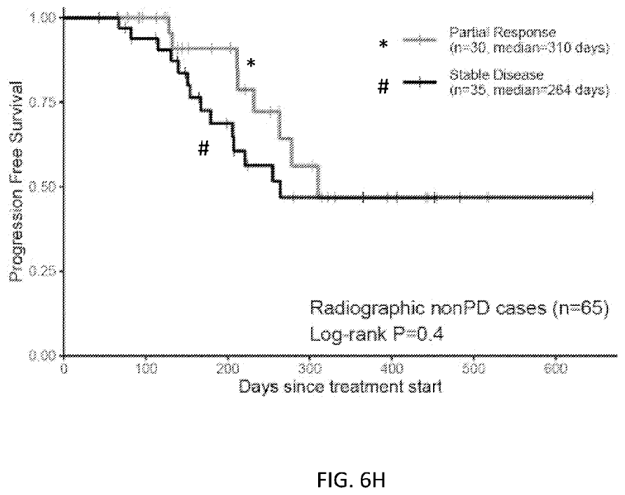

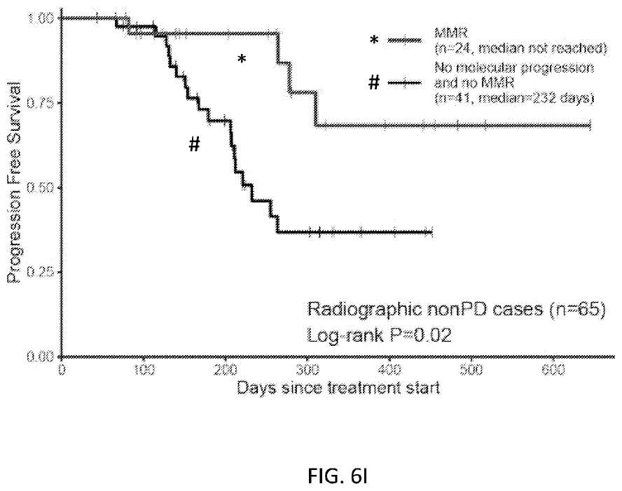

FIGS. 6A-6I show molecular response assessment early in the course of therapy was associated with favorable PFS, in accordance with some embodiments. FIG. 6A shows that the full cohort (n=92) had median PFS of 211 days. FIG. 6B shows that patients with a molecular progression detected from cfDNA at T1 or T2 (n=14, median PFS=62 days) had significantly worse PFS compared to patients with no molecular progression (n=78, median PFS=263 days; HR=12.6 [95% CI: 5.8 to 27.3]; log-rank P<1E-10). FIGS. 6C-6 D show subset analysis based on therapeutic modality for patients on immunotherapy with or without chemotherapy (n=34; log-rank P=2E-12) (FIG. 6C), patients on chemotherapy with or without targeted therapy (n=42; log-rank P=7E-6) (FIG. 6D). FIGS. 6E-6F show subset analysis based on cancer type for lung cancer patients (n=40; log-rank P=8E-8) (FIG. 6E) and breast cancer patients (n=25; log-rank P=3E-4) (FIG. 6F). FIG. 6G shows that patients with a MMR had significantly longer PFS after accounting for predictions based on molecular progression (Cox P=0.011). FIGS. 6H-6I show subset analysis for patients with either stable disease or partial response determined by radiography at first FUI (n=65) stratified by response status (log-rank P=0.4) (FIG. 6H) or MMR (log-rank P=0.02) (FIG. 61).

Molecular response pattern assessed by cfDNA early on treatment was correlated with PFS. The median PFS for the full cohort of patients was 211 days (FIG. 6A). Patients with molecular progression at either T1 or T2 (n=14) had shorter PFS, with HR=12.6 (95% CI of 5.8 to 27.3; Log-rank P=7E-16) and median PFS of 63 days, as compared to 263 days for patients with no molecular progression (n=78) (FIG. 6B).

The predictive performance of the assay was explored in subsets of patients based on tumor origin and treatment type. Among the 34 patients who received immunotherapy, PFS was significantly shorter in patients with molecular progression (median PFS=57 days) compared to patients without identified molecular progression (median PFS not reached after a median of 167 days follow-up, log-rank P=2E-12), consistent with the overall cohort (FIG. 6C). The results were similar for the chemotherapy subset, with a median PFS of 56 days in patients with molecular progression and 212 days without progression (FIG. 6D; n=42; log-rank P=7E-6).

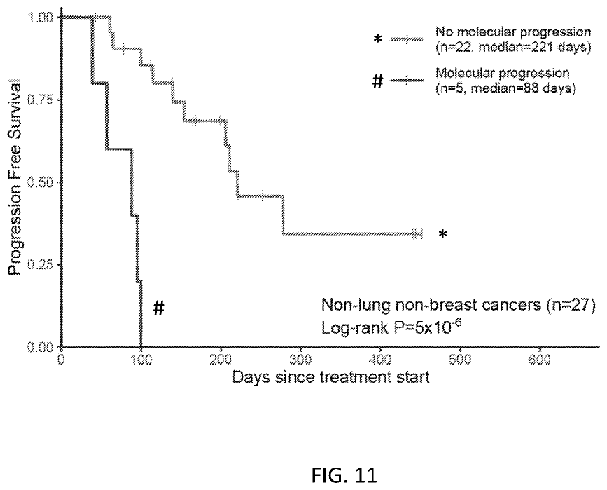

PFS was also significantly shorter for patients with molecular progression for the two largest subsets, lung cancer (FIG. 6E; log-rank P=8E-8) and breast cancer (FIG. 6F; log-rank P=3E-4). This was also true for the remaining subset of mixed cancer types (gastrointestinal, genitourinary, melanoma, and sarcoma) (log-rank P=5E-6). FIG. 11 shows molecular response assessment and PFS for other cancers, in accordance with some embodiments. This figure shows non-lung non-breast cancers (n=27; log-rank P=5E-6), plotted as in FIGS. 6E-6F. Predictions of progression at first FUI also had comparable performance across these subsets (Table S4). Together, these data indicate that the assay was effective across multiple different cancer types and treatment modalities.

TABLE S4
Molecular progression and assessment at first
FUI for cancer and treatment type subsets

| | | ctDNA results | |
|---|---|---|---|
| | Assessment | Molecular | No molecular |
| Patient subset | at FUI | Progression | progression |
| Immunotherapy | PD | 5 | 2 |
| (+/−Chemotherapy) | nonPD | 0 | 27 |
| Chemotherapy | PD | 4 | 7 |
| (+/−Targeted therapy) | nonPD | 0 | 31 |
| Lung cancer | PD | 4 | 4 |
| | nonPD | 0 | 32 |
| Breast cancer | PD | 5 | 5 |
| | nonPD | 0 | 15 |
| Non-lung non-breast | PD | 5 | 3 |
| cancers | nonPD | 0 | 19 |

Further, results demonstrated that a large quantitative reduction early in the treatment course was associated with an improved PFS. Of the 78 patients for whom the assay did not show molecular progression, 27 patients had an MMR at either post-treatment time point. Notably, in all cases where an MMR was identified at T1, this finding was also observed at T2, if that time point was available (n=12). In 7 cases, the TFR from baseline did not reach a 10-fold reduction at T1 but did so at T2, for example for patient LS030093 (FIG. 4E). Patients with an MMR had longer PFS (FIG. 6G, median PFS not reached) compared to patients with no MMR and no molecular progression (n=51; median PFS of 211 days). An MMR was significantly predictive of PFS after accounting for the predictions based on molecular progression (Stratified Cox: molecular progression P=4E-9, MMR P=0.011), indicating value in the early quantitative ctDNA dynamics for predicting longer term treatment efficacy.

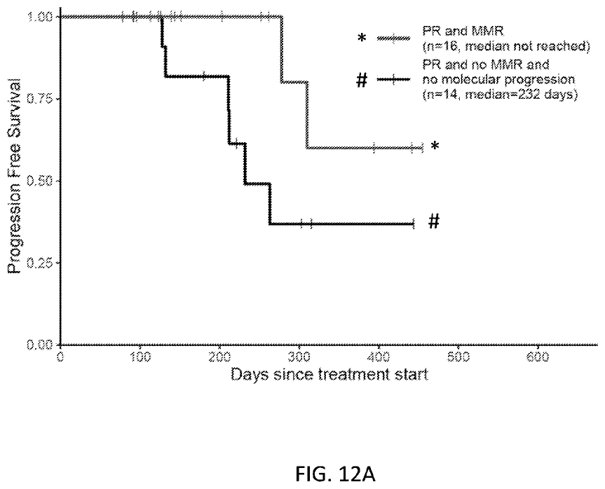

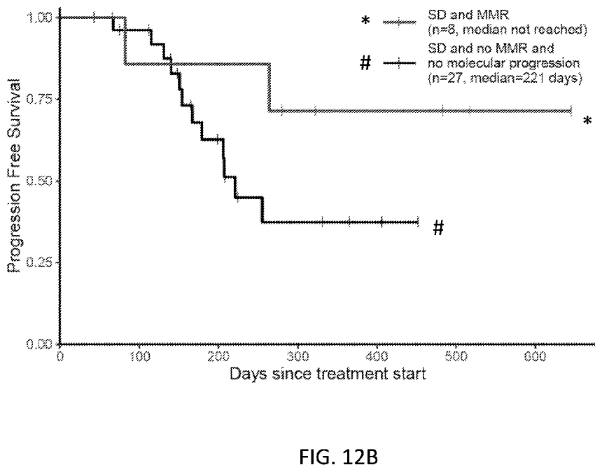

Next, the predictive value of an MMR in conjunction with radiographic response monitoring was assessed. For patients who did not show radiographic progression at first FUI (n=65), RECIST 1.1 based partial response vs. stable disease at first FUI had limited prognostic value for PFS (FIG. 6H; log-rank P=0.4; HR=0.67 [95% CI of 0.28 to 1.60]). However, among the same subset, patients with an MMR had substantially longer PFS (FIG. 61; log-rank P=0.02; HR=0.28 [95% CI of 0.09 to 0.84]), which was also significant after adjusting for radiographic assessment at first FUI (Stratified Cox: partial response P=0.36, MMR P=0.016). FIGS. 12A-12B show MMR and PFS for patients with non-PD at first FUT, in accordance with some embodiments. These figures show results from all patients with radiographic partial response (n=30) (FIG. 12A) or stable disease (n=35) (FIG. 12B).

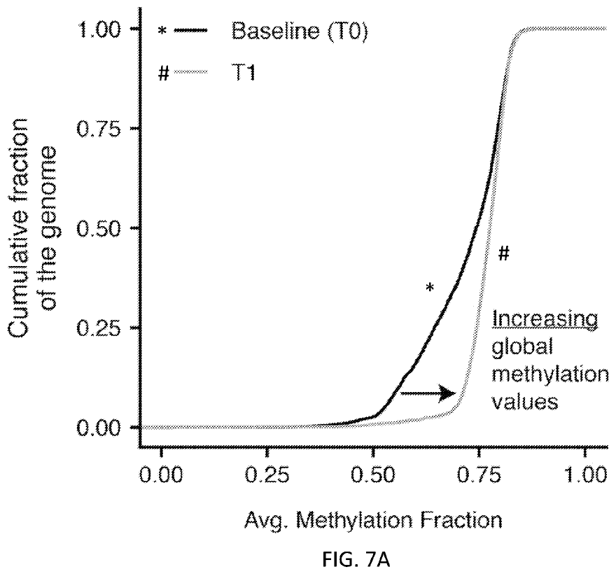

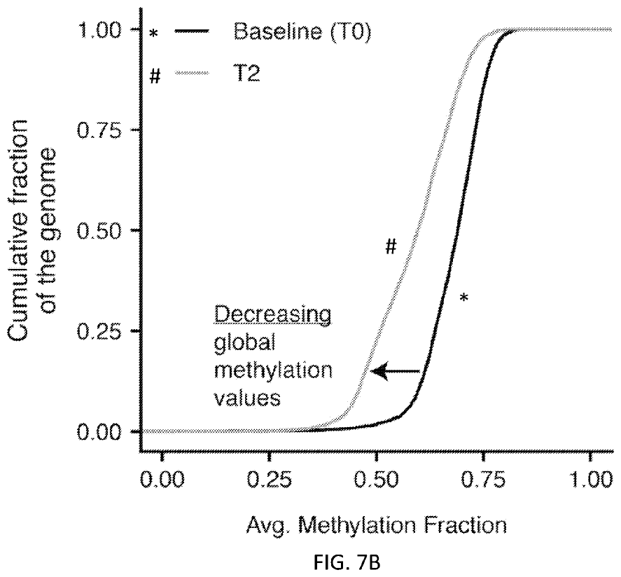

Longitudinal changes in methylation levels may complement tumor fraction changes. Global hypomethylation may be a hallmark of tumor genomes, and an increase in global methylation levels in cfDNA may be associated with non-progression, as it may indicate a decreased proportion of ctDNA. Importantly, the epigenetic patterns observed in tumors, including overall global hypomethylation, can be detected in ctDNA. To assess the potential of methylation changes for identifying early response to therapy, the change in genome-wide methylation levels from baseline to post-treatment was examined retrospectively for two example patients (FIGS. 7A-7B)—one with a non-PD call (LS030083) at first FUI and one with a PD call at first FUI (LS030078). Consistent with the clinical assessment at first FUT, a marked increase in methylation levels was observed for LS030083 (FIG. 7A), while a decrease was observed in LS030078 (FIG. 7B). For patient LS030078, a clear molecular progression was observed based on CNAs and local fragmentation changes (TFR=2.01), while no CNAs were detectable in LS030083.

FIGS. 7A-7B show that methylation may provide an orthogonal signal to CNAs for response monitoring, in accordance with some embodiments. These figures show a distribution of average methylation levels in genome-wide 1 megabase bins for patient LS030083 (FIG. 7A) and LS030078 (FIG. 7B) at baseline (black line) and either T1 or T2 (orange line).

Patients with advanced malignancies may require careful treatment monitoring to assess therapeutic efficacy, promote quality of life, and limit drug toxicity. However, current methods for disease monitoring using clinical and radiographic assessment may require several months to confidently determine treatment response. Here, the utility of an improved, whole-genome cfDNA molecular response assay was assessed, which analyzed longitudinal ctDNA measurements at baseline and during the initial cycles of treatment to predict response to therapy. This technique predicted disease progression with both a specificity and positive predictive value (PPV) of 100%, indicating that this assay was able to predict disease progression with high confidence and reliability at a median of 6 weeks earlier than current standard of care methods. This finding indicates that advanced tumors that are progressing early in the course of treatment may be reflected as an observable signal in the blood well before visible change in the size, contour, or density of target lesions that can be detected by current imaging schedule and interpretation.

These results indicate several other key findings. First, the blood-based, whole-genome ctDNA molecular assay appeared to predict disease progression consistently across a variety of tumor and treatment types, by looking beyond targeted assessment of tumor-specific point mutations and fusions. Specifically, subset analyses of the two cohorts with the largest number of patients (lung and breast cancer) demonstrated statistically significant findings, which were also observed collectively across non-lung and non-breast patients. Similar predictive value was encountered across different treatments, including patients who received chemotherapy or immunotherapy.

These findings indicate that methods and systems of the present disclosure represent an improved CNA-based approach for certain cohorts of patients (e.g., those treated with immunotherapy alone). Second, results demonstrated that patients with an MMR had a longer PFS compared to those with no change or a smaller decrease in TFR, indicating that there was quantitative value in the degree of initial response to therapy. In the data obtained, PFS was more strongly associated with the degree of response measured by the assay, as compared with RECIST 1.1 classification of partial response vs. stable disease, indicating that radiographic assessment of partial response vs. stable at first FUI may have limited long-term prognostic value in some cases. The additional prognostic value of identifying an MMR supports the potential to integrate imaging and analysis of serial cfDNA samples to provide an early indication of an extended duration of disease control.

Molecular response monitoring with serial measurements of cfDNA has potential clinical benefit for both assessing disease control and disease progression. For example, if MMR is observed, early reassurance that the current treatment regimen is effective may be used to limit the frequency of clinical imaging. In contrast, an early prediction of blood-based molecular progression may guide oncologists to discontinue ineffective treatments, thereby reducing avoidable side effects and financial toxicity. By accelerating the clinical decision loop, patients may be afforded the opportunity to change to an alternative, potentially effective therapy. This assessment algorithm may increase the availability of patients with adequate performance status to engage in multiple lines of therapy, including clinical trials. Furthermore, a blood-based assay provides convenience for patients as blood samples are collected routinely during each cycle of therapy.

While the specificity of the assay was very high, which is the critical performance metric for clinical utility in the advanced setting, the sensitivity, particularly at the earliest time point, may be improved by including other features such as cancer-associated epigenetic signals. For example, in patient LS030083, there was a marked increase in genome-wide methylation levels from baseline to post-treatment, consistent with a non-PD call at first FUT, yet no CNAs were detected (FIG. 7A). Therefore, these methylation-based signals may be incorporated into the assay, along with fragment length and copy number information, to increase assay sensitivity for samples with low tumor fraction. However, even with additional orthogonal signals, there may be a residual false negative rate from tumors that do not generate sufficient ctDNA (e.g., non-shedding tumors), tumors that have not yet progressed during the earliest cycles of treatment, and/or tumors in which molecular progression by ctDNA analysis and imaging are not in agreement. Further, expanded patient cohorts may be used to confirm the tumor and treatment-agnostic nature of the assay. Further, prospective validation of the prediction tool may be performed. An independent validation cohort may be examined to assess how treatment changes using an adaptive clinical trial design may both limit cost and improve long-term patient outcomes by switching to different treatment arms faster using the blood-based molecular response assessment.

In conclusion, the results demonstrated that a serial monitoring approach analyzing whole-genome ctDNA with low-pass sequencing produced clinical predictions with a high degree of specificity and PPV compared with standard of care clinical assessment. In addition, the findings were consistent across multiple tumor and treatment types, and the degree of ctDNA reduction correlated with long-term clinical outcome. This non-invasive tool may more accurately match advanced cancer patients with potentially effective therapies earlier, thereby limit side effects and costs associated with ineffective treatments.

### Example 2

### Using cfDNA Methylation and Fragment Length to Track Treatment Response

Using methods and systems of the present disclosure, models were developed based on analyzing fragments of cfDNA to understand patient response to cancer therapy.

In general, fragments of tumor derived cfDNA (ctDNA) are shorter than cfDNA from non-cancerous tissue. ctDNA also has lower overall levels of methylation. Therefore, models were developed to use this information to corroborate calls of copy-number aberrations (CNAs).

The extent to which the methylation patterns agree with the CNA calls and the way in which the fragment length patterns agree with the CNA calls were assessed. In practice, this is done for each library from a subject (e.g., a patient) by quantifying the correlation between the average methylation and/or fragment length in all the genomic bins (e.g., every 500 kilobase region in the genome) and the copy number in that region.

In true cancer patients with correctly called CNAs, an expected observation is an anticorrelation between the copy number of the tumor and both the average methylation fraction and the average fragment length. This is because in regions with a copy number gain (e.g., the copy number in the tumor is 3 or greater, compared to 2 copies in all the non-tumor cells), a larger fraction of the cfDNA in the blood is tumor-derived. In contrast, in a region of the genome where the tumor has only one copy, a higher fragment length and methylation are expected, because a smaller fraction of the cfDNA is derived from the tumor cells.

These correlation coefficients, as measured by Spearman's rho (or alternatively, another statistical test of correlations, such as Pearson's R), are an independent approach of assessing whether or not a particular set of CNA calls for a sample is genuinely tumor-derived as opposed to arising due to noise in the sequencing data. This allows for an improved specificity of CNA calls, thereby resulting in fewer false positives, and ultimately a more effective approach of performing longitudinal monitoring of a patient's response or non-response to therapy.

Therefore, using methods and systems of the present disclosure, fragment length and/or methylation data for cfDNA were leveraged for improving the specificity of CNA calls.

### Example 3

### Monitoring Tumor Progression Based on Combined Whole Genome and Methylation Signals of cfDNA

Using methods and systems of the present disclosure, tumor progression is performed based on a combination of two signals obtained from whole genome sequencing and methylation-aware sequencing (methyl-seq) of cfDNA samples. For example, a combined score for tumor progression may be calculated based on enzymatic methyl-seq analysis via the following approach.

First, a CNA-based tumor fraction ratio (TFR) is determined. This may be done by analyzing a first cfDNA sample from a patient at a baseline time point and a second cfDNA sample from the patient at a subsequent follow-up time point, and then comparing the read depth libraries from the follow-up time point to the patient's baseline time point.

Next, a second cancer-associated signal is determined based on methylation analysis, as described in Example 4. Next, the whole genome and methylation signals are combined into a combined prediction of the fold change in tumor fraction between the first follow-up timepoint and the baseline time point. The whole genome and methylation signals may be combined using a variety of methods, including using a logistic regression, using a weighted average of the log-transformed values (e.g., equivalent to a geometric mean), or a weighted average taking into account the estimated statistical precision of each measure in the particular patient's profile.

For ease of use, the combined ratio may be normalized, scaled, or transformed into a combined score on a convenient scale, such as a scale of 0 to 200. For example, this may be performed by using the following transformation: score=max_score/(1+1/ratio). A score of 100 is the baseline score, a higher score indicates a worse outcome (e.g., more tumor progression and lower molecular response), and a lower score indicates a better outcome (e.g., less or no tumor progression, and higher molecular response). Based on this scoring assessment, patients are assigned to one of three categorizations: molecular progression, major molecular response (MMR), and no-progression or no major molecular response.

Molecular progression (MP) is defined as a statistically significant increase in tumor fraction, while MMR may be defined as a significant (e.g., at least 10X) decrease in tumor fraction from the baseline time point to a follow-up time point.

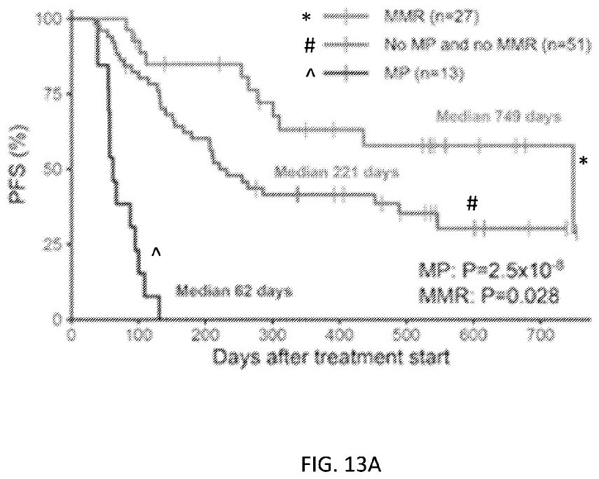

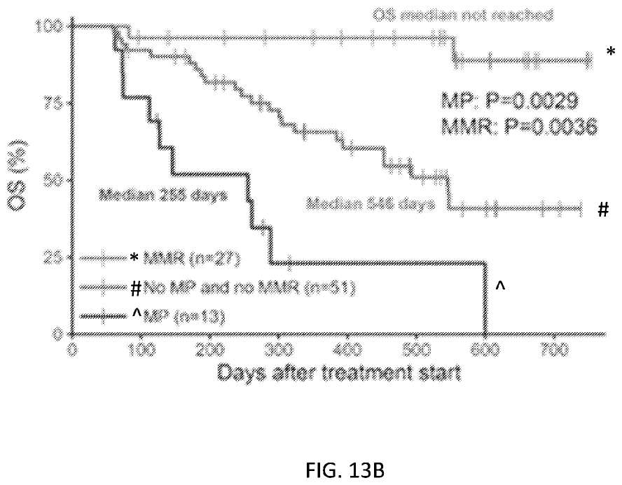

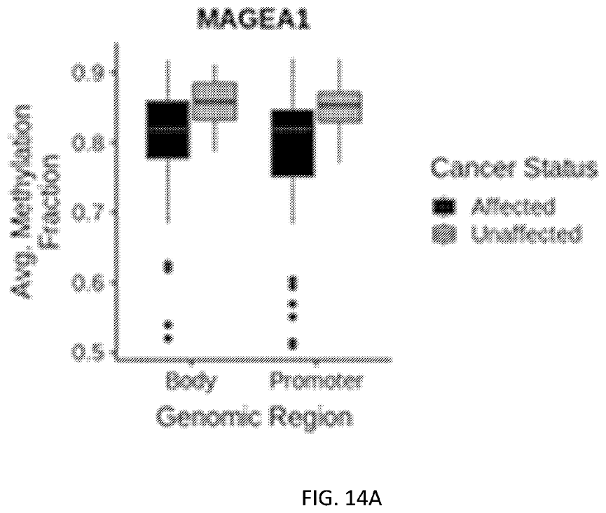

FIGS. 13A-13B show examples of Kaplan-Meier progression free survival (PFS) and overall survival (OS) plots for each of these three patient categories (MP, MMR, and neither MP nor MMR) in the patient cohort. These figures show that the survival curves are highly separated from each other. Furthermore, the predictions of molecular progression predict radiographic progression with high specificity.

### Example 4

### Monitoring Tumor Progression Based on a Methylation Signal of cfDNA

Using methods and systems of the present disclosure, a variety of different approaches can be used for extracting cancer-associated signals from methylation profiles of cfDNA samples, and monitoring tumor progression based on the cancer-associated signals. For example, such approaches may comprise measuring signals from methylation fraction in CpG islands, shores, PMD, promoters, gene bodies, repeat elements, known cancer genes, and single CpG sites. In particular, the incorporation of CNA data from cfDNA into such tumor progression methods was demonstrated to advantageously improve monitoring of tumor progression with increased sensitivity (as compared to using whole genome data), by training a methylation tumor fraction model using linear regression with strong regularization (or another sort of model) on a set of samples with known tumor fraction by the orthogonal method of CNA calling.

The methylation signal extraction may comprise identifying all the libraries where the tumor fraction of a given cfDNA sample from a cancer patient, or the fact that the sample is from an unaffected control subject, can be confidently determined from the CNA pattern.

Next, for each such library, an average methylation fraction in all of the CpG islands in the genome (or alternatively, another class of region, such as shores or promoters) is determined from the methylation sequencing data. The methylation sequencing data may be generated by, for example, whole genome bisulfite sequencing or enzymatic methyl-sequencing of cfDNA samples.

Next, a regression or modeling is performed (e.g., a linear regression, simple regression, binary regression, Bayesian linear regression, polynomial regression, Gaussian process regression, binary regression, logistic regression, nonlinear regression, etc.) with regularization of the known tumor fractions against the methylation patterns using a suitable cross validation approach (e.g., leave-one-participant-out cross validation). From these results, a prediction of the tumor fraction of the cfDNA sample can be generated based on the methylation pattern, using methods and systems of the present disclosure.

The methylation signal approach yields an estimate of the methylation fraction in cfDNA, even in cases where a CNA signal is low or undetectable. The methylation signal is then used in a combined model for scoring (e.g., as described in Example 3), and/or compared between timepoints (e.g., a baseline time point and one or more subsequent follow-up time points).

Other approaches may be used to extract a cancer-associated methylation signal from cfDNA samples. For example, the weights may be computed using principal component analysis (e.g., the first, most significant principal component may be observed to be highly associated with cancer or possibly a subsequence principal component depending on what other variations are present in the data). As another example, sequencing reads may be filtered based on fragment length before applying principal component analysis, thus enriching for tumor-derived reads. As another example, methylation haplotype load (MHL) may be determined in methylation haplotype blocks, and an inverse MHL (similar to MHL, but for unmethylated blocks) may be computed. Any one or combination of these approaches may be used to generate a cancer-associated methylation signal from sequencing or methyl-seq data. Any one or a combination of these could be used as inputs to the tumor fraction modeling described herein.

### Example 5

### Using cfDNA to Predict Tumor Gene Expression Toward Prediction of Therapy Response

Methylation may play a strong role in regulating gene expression. Generally, it may be observed that methylation in gene promoter regions suppresses gene expression. As an example, an aspect of aberrant methylation in cancer is the loss of normal high levels of oncogene promoter methylation, which may lead to over-expression of the oncogenes and hence result in a more cancerous state. In particular, the MAGE (melanoma-associated antigen) family of genes is archetypal of this aberrant methylation in many cancer types. Therefore, the ability to test for gene over-expression via analysis of the methylation state of the cfDNA of a subject by liquid biopsy may be advantageous, such as in cases in which drug candidates are being developed to target one of such over-expressed oncogenes.

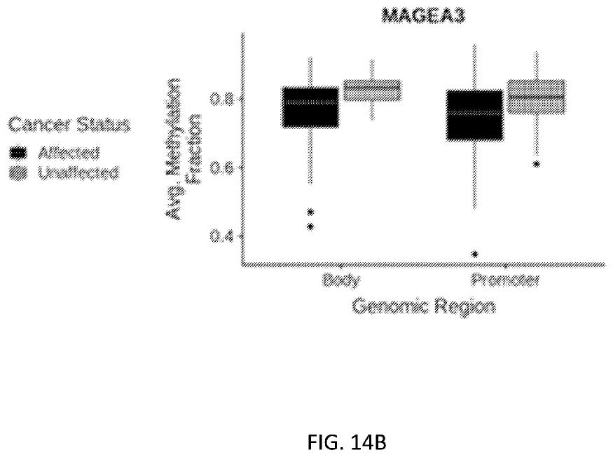

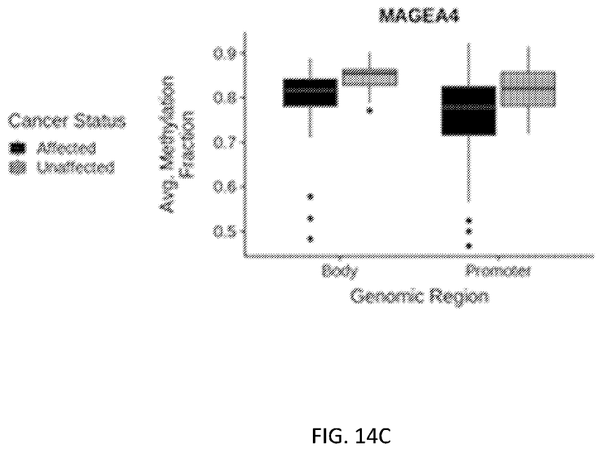

Using methods and systems of the present disclosure, the total methylation levels averaged over tumor and healthy tissue of the genes of interest are measured via a liquid biopsy analysis of a biological sample of a subject. In the case of the MAGE genes, the methylation level is known to be constitutively high in all normal adult tissues other than testis. Therefore, when reduced methylation is observed at MAGE promoters, this is likely indicative of the tumor of the subject. FIGS. 14A-14C show examples of a strong average decrease in methylation observed at three MAGE genes (MAGEA1, MAGEA3, and MAGEA4). As seen in FIGS. 14A-14C, the strong average decrease in methylation was observed at the three different MAGE genes in a plurality of patients, which exceeded the genome-wide level of hypomethylation. This result indicates that for patients with sufficient circulating tumor DNA (ctDNA) in the blood (e.g., cfDNA of tumor origin), there is a set of genes which was methylation-repressed in normal tissue. For this set of genes, hypomethylation and expression in the tumor can be analyzed and measured using methods and systems of the present disclosure.

Overall, tumor cells may exhibit aberrant hypomethylation of germline-expressed genes, such as the promoters of MAGE, which leads to their over-expression, thereby resulting in poor consequences, outcomes and prognoses for cancer patients. Using methods and systems of the present disclosure, the hypomethylation of these genes was detected, which allows the individualized selection of targeted therapies that target these genes via a liquid biopsy (e.g., without requiring a tumor tissue biopsy or other invasive assay).

Methods and systems of the present disclosure can be implemented by way of one or more algorithms. An algorithm can be implemented by way of software upon execution by the central processing unit 205. The algorithm can, for example, process WGS data to determine copy number aberrations (CNAs) and fragment lengths of cfDNA molecules, process CNAs to determine a CNA profile change, process fragment lengths to determine a fragment length profile change by quantifying the change in strength of a specific CNA signal in multiple samples from the patient over the course of treatment. As shown in FIGS. 15A & 15B, such approaches can be less prone to certain error modes arising from separately quantifying tumor fractions in separate samples based on CNAs.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for assessing tumor status of a subject with cancer, comprising:
obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;
determining, based on the first WGS data, (i) a first plurality of copy number aberrations (CNAs) in the first plurality of cfDNA molecules and (ii) a first plurality of fragment lengths of the first plurality of cfDNA molecules;
obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;
determining, based on the second WGS data, (iii) a second plurality of copy number aberrations (CNAs) in the second plurality of cfDNA molecules and (iv) a second plurality of fragment lengths of the second plurality of cfDNA molecules;
comparing the first plurality of CNAs with the second plurality of CNAs to determine a CNA profile change;
determining a fragment length profile change based on the first plurality of fragment lengths and the second plurality of fragment lengths;
determining a first tumor fraction of the subject at the first timepoint or a second tumor fraction of the subject at the second timepoint, based at least in part on the CNA profile change and the fragment length profile change; and
detecting a tumor status of the subject based at least in part on the first tumor fraction or the second tumor fraction.

2. The method of claim 1, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.

3. The method of claim 1, wherein obtaining the first WGS data comprises sequencing the first plurality of cfDNA molecules to generate a first plurality of sequencing reads, or wherein obtaining the second WGS data comprises sequencing the second plurality of cfDNA molecules to generate a second plurality of sequencing reads.

4. The method of claim 3, wherein the sequencing is performed at a depth of no more than about 25×.

5. The method of claim 3, wherein the sequencing is performed at a depth of no more than about 10×.

6. The method of claim 3, wherein the sequencing is performed at a depth of no more than about 8×.

7. The method of claim 3, wherein the sequencing is performed at a depth of no more than about 6×.

8. The method of claim 3, further comprising aligning the first or second plurality of sequencing reads to a reference genome, thereby producing a plurality of aligned sequencing reads.

9. The method of claim 1, further comprising enriching the first or second plurality of cfDNA molecules for a plurality of genomic regions.

10. The method of claim 9, wherein the enrichment comprises amplifying the first or second plurality of cfDNA molecules.

11. The method of claim 10, wherein the amplification comprises selective amplification.

12. The method of claim 10, wherein the amplification comprises universal amplification.

13. The method of claim 9, wherein the enrichment comprises selectively isolating at least a portion of the first or second plurality of cfDNA molecules.

14. The method of claim 13, wherein selectively isolating the at least the portion of the first or second plurality of cfDNA molecules comprises using a plurality of probes, each of the plurality of probes having sequence complementarity with at least a portion of a genomic region of the plurality of genomic regions.

15. The method of claim 13, wherein the at least the portion comprises a tumor marker locus.

16. The method of claim 15, wherein the at least the portion comprises a plurality of tumor marker loci.

17. The method of claim 16, wherein the plurality of tumor marker loci comprises one or more loci selected from The Cancer Genome Atlas (TCGA) or Catalogue of Somatic Mutations in cancer (COSMIC).

18. The method of claim 3, wherein determining the first plurality of CNAs comprises determining quantitative measures of CNAs at each of at each of a plurality of genomic regions of the first plurality of sequencing reads, and wherein determining the second plurality of CNAs comprises determining quantitative measures of CNAs at each of at each of the plurality of genomic regions of the second plurality of sequencing reads.

19. The method of claim 18, further comprising correcting the first plurality of CNAs or the second plurality of CNAs for GC content and/or mappability bias.

20. The method of claim 19, wherein the correcting comprises using a statistical modeling analysis.

21. The method of claim 20, wherein the statistical modeling analysis comprises LOESS regression or a Bayesian model.

22. The method of claim 18, wherein the plurality of genomic regions comprises non-overlapping genomic regions of a reference genome having a pre-determined size.

23. The method of claim 22, wherein the pre-determined size is about 50 kilobases (kb), about 100 kb, about 200 kb, about 500 kb, about 1 megabases (Mb), about 2 Mb, about 5 Mb, or about 10 Mb.

24. The method of claim 18, wherein the plurality of genomic regions comprises at least about 1,000 distinct genomic regions.

25. The method of claim 24, wherein the plurality of genomic regions comprises at least about 2,000 distinct genomic regions.

26. The method of claim 1, wherein determining the CNA profile change comprises comparing the first plurality of CNAs and the second plurality of CNAs with a plurality of reference CNA values, wherein the plurality of reference CNA values is obtained from additional cfDNA molecules obtained or derived from additional bodily fluid samples of additional subjects.

27. The method of claim 26, wherein the additional subjects comprise one or more subjects without cancer.

28. The method of claim 26, wherein the additional subjects comprise one or more subjects not having tumor progression.

29. The method of claim 26, wherein the plurality of reference CNA values is obtained using additional bodily fluid samples of the subject obtained at one or more subsequent time points after the first timepoint.

30. The method of claim 1, further comprising filtering out a subset of the first plurality of CNAs and the second plurality of CNAs that meet a pre-determined criterion.

31. The method of claim 30, further comprising filtering out a given CNA value of the first plurality of CNAs or the second plurality of CNAs values when the difference between the given CNA value and the corresponding reference CNA value comprises a difference of no more than about 1 standard deviation.

32. The method of claim 31, further comprising filtering out a given CNA value of the first plurality of CNAs or the second plurality of CNAs values when the difference between the given CNA value and the corresponding reference CNA value comprises a difference of no more than about 2 standard deviations.

33. The method of claim 31, further comprising filtering out a given CNA value of the first plurality of CNAs or the second plurality of CNAs values when the difference between the given CNA value and the corresponding reference CNA value comprises a difference of no more than about 3 standard deviations.

34. The method of claim 30, further comprising filtering out a given CNA value of the first plurality of CNAs or the second plurality of CNAs values based on a Spearman's rank correlation between the given CNA value and a corresponding local mean fragment length.

35. The method of claim 34, further comprising filtering out a given CNA value of the first plurality of CNAs or the second plurality of CNAs values when the Spearman's rank correlation coefficient (Spearman's rho) is less than −0.1.

36. The method of claim 1, further comprising normalizing the first plurality of fragment lengths or the second plurality of fragment lengths based on a library or a genomic location.

37. The method of claim 1, further comprising detecting that the tumor status comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5.

38. The method of claim 1, further comprising detecting a major molecular response (MMR) of the subject when the first tumor fraction or the second tumor fraction is less than 0.01, less than 0.05, less than 0.1, less than 0.2, less than 0.3, less than 0.4, or less than 0.5.

39. The method of any one of claims 1-38, further comprising detecting the tumor status of the subject with a sensitivity of at least about 50%.

40. The method of claim 39, further comprising detecting the tumor status of the subject with a sensitivity of at least about 70%.

41. The method of claim 40, further comprising detecting the tumor status of the subject with a sensitivity of at least about 90%.

42. The method of any one of claims 1-41, further comprising detecting the tumor status of the subject with a specificity of at least about 50%.

43. The method of claim 42, further comprising detecting the tumor status of the subject with a specificity of at least about 70%.

44. The method of claim 43, further comprising detecting the tumor status of the subject with a specificity of at least about 90%.

45. The method of claim 44, further comprising detecting the tumor status of the subject with a specificity of at least about 98%.

46. The method of any one of claims 1-45, further comprising detecting the tumor status of the subject with a positive predictive value (PPV) of at least about 50%.

47. The method of claim 46, further comprising detecting the tumor status of the subject with a positive predictive value (PPV) of at least about 70%.

48. The method of claim 47, further comprising detecting the tumor status of the subject with a positive predictive value (PPV) of at least about 90%.

49. The method of any one of claims 1-48, further comprising detecting the tumor status of the subject with a negative predictive value (NPV) of at least about 50%.

50. The method of claim 49, further comprising detecting the tumor status of the subject with a negative predictive value (NPV) of at least about 70%.

51. The method of claim 50, further comprising detecting the tumor status of the subject with a negative predictive value (NPV) of at least about 90%.

52. The method of any one of claims 1-51, further comprising detecting the tumor status of the subject with an area under the curve (AUC) of at least about 0.60.

53. The method of claim 52, further comprising detecting the tumor status of the subject with an area under the curve (AUC) of at least about 0.75.

54. The method of claim 53, further comprising detecting the tumor status of the subject with an area under the curve (AUC) of at least about 0.90.

55. The method of any one of claims 1-54, further comprising determining a tumor non-progression of the subject when tumor progression is not detected.

56. The method of any one of claims 1-55, further comprising, based on the determined tumor status of the subject, administering a therapeutically effective dose of a treatment to treat the cancer of the subject.

57. The method of claim 56, wherein the treatment comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor.

58. The method of any one of claims 1-57, wherein the detected tumor status is indicative of tumor progression, non-progression, regression, or recurrence.

59. The method of any one of claims 1-58, wherein the first and second WGS data are obtained by pyrosequencing, sequencing-by-synthesis, single-molecule sequencing, Nanopore sequencing, semiconductor sequencing, sequencing-by-ligation, sequencing-by-hybridization, massively parallel sequencing, chain termination sequencing, single molecule real-time sequencing, Polony sequencing, combinatorial probe anchor synthesis, or hybrid capture-based sequencing.

60. The method of any one of claims 1-59, wherein the first and second WGS data are obtained by a sequencing device or computer processor.

61. A computer system for assessing tumor status of a subject with cancer, comprising:
a database that is configured to store (i) first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject, and (ii) second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject; and
one or more computer processors operatively coupled to the database, wherein the one or more computer processors are individually or collectively programmed to:
determine, based on the first WGS data, (i) a first plurality of copy number aberrations (CNAs) in the first plurality of cfDNA molecules and (ii) a first plurality of fragment lengths of the first plurality of cfDNA molecules;
determine, based on the second WGS data, (iii) a second plurality of copy number aberrations (CNAs) in the second plurality of cfDNA molecules and (iv) a second plurality of fragment lengths of the second plurality of cfDNA molecules;
compare the first plurality of CNAs with the second plurality of CNAs to determine a CNA profile change;
determine a fragment length profile change based on the first plurality of fragment lengths and the second plurality of fragment lengths;
determine a first tumor fraction of the subject at the first timepoint or a second tumor fraction of the subject at the second timepoint, based at least in part on the CNA profile change and the fragment length profile change; and
detect a tumor status of the subject based at least in part on the first tumor fraction or the second tumor fraction.

62. A non-transitory computer-readable medium comprising machine-executable instructions which, upon execution by one or more computer processors, perform a method for assessing tumor status of a subject with cancer, the method comprising:
obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;
determining, based on the first WGS data, (i) a first plurality of copy number aberrations (CNAs) in the first plurality of cfDNA molecules and (ii) a first plurality of fragment lengths of the first plurality of cfDNA molecules;
obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;
determining, based on the second WGS data, (iii) a second plurality of copy number aberrations (CNAs) in the second plurality of cfDNA molecules and (iv) a second plurality of fragment lengths of the second plurality of cfDNA molecules;
comparing the first plurality of CNAs with the second plurality of CNAs to determine a CNA profile change;
determining a fragment length profile change based on the first plurality of fragment lengths and the second plurality of fragment lengths;
determining a first tumor fraction of the subject at the first timepoint or a second tumor fraction of the subject at the second timepoint, based at least in part on the CNA profile change and the fragment length profile change; and
detecting a tumor status of the subject based at least in part on the first tumor fraction or the second tumor fraction.

63. A method for assessing tumor status of a subject with cancer, comprising:
obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;
determining, based on the first MS data, an average methylation fraction for each of one or more CpG islands in the region of the genome, thereby obtaining a first average methylation fraction profile;
obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;
determining, based on the second MS data, an average methylation fraction for each of one or more CpG islands in the region of the genome, thereby obtaining a second average methylation fraction profile;
comparing the first average methylation fraction profile across the one or more CpG islands and the second average methylation fraction profile across the one or more CpG islands to determine a methylation fraction profile;
determining a first tumor fraction of the subject at the first timepoint or a second tumor fraction of the subject at the second timepoint, based at least in part on the respective methylation fraction profiles; and
detecting a tumor status of the subject based at least in part on the first tumor fraction or the second tumor fraction.

64. The method of claim 63, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.

65. The method of claim 63, wherein obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads.

66. The method of claim 65, wherein the methylation sequencing comprises whole genome bisulfite sequencing.

67. The method of claim 65, wherein the methylation sequencing comprises whole genome enzymatic methyl-seq.

68. The method of claim 65, wherein the methylation sequencing comprises oxidative bisulfite sequencing, TET-assisted pyridine borane sequencing (TAPS), TET-assisted bisulfite sequencing (TABS), oxidative bisulfite sequencing (oxBS-Seq), APOBEC-coupled epigenetic sequencing (ACE-seq), methylated DNA immunoprecipitation (MeDIP) sequencing, hydroxymethylated DNA immunoprecipitation (hMeDIP) sequencing, methylation array analysis, reduced representation bisulfite sequencing (RRBS-Seq), or cytosine 5-hydroxymethylation sequencing.

69. The method of claim 65, wherein the methylation sequencing is performed at a depth of no more than about 25×.

70. The method of claim 65, wherein the methylation sequencing is performed at a depth of no more than about 10×.

71. The method of claim 65, wherein the methylation sequencing is performed at a depth of no more than about 8×.

72. The method of claim 65, wherein the methylation sequencing is performed at a depth of no more than about 6×.

73. The method of claim 65, further comprising aligning the first or second plurality of sequencing reads to a reference genome, thereby producing a plurality of aligned sequencing reads.

74. The method of claim 65, further comprising enriching the first or second plurality of cfDNA molecules for the region of the genome.

75. The method of claim 74, wherein the enrichment comprises amplifying the first or second plurality of cfDNA molecules.

76. The method of claim 75, wherein the amplification comprises selective amplification.

77. The method of claim 75, wherein the amplification comprises universal amplification.

78. The method of claim 74, wherein the enrichment comprises selectively isolating at least a portion of the first or second plurality of cfDNA molecules.

79. The method of claim 78, wherein selectively isolating the at least the portion of the first or second plurality of cfDNA molecules comprises using a plurality of probes, each of the plurality of probes having sequence complementarity with at least a portion of the region of the genome.

80. The method of claim 78, wherein the at least the portion comprises a tumor marker locus.

81. The method of claim 80, wherein the at least the portion comprises a plurality of tumor marker loci.

82. The method of claim 81, wherein the plurality of tumor marker loci comprises one or more loci selected from The Cancer Genome Atlas (TCGA) or Catalogue of Somatic Mutations in cancer (COSMIC).

83. The method of claim 63, wherein the region of the genome comprises one or more of: CpG islands, CpG shores, patient-specific partially methylated domains, common partially methylated domains, promoters, gene bodies, evenly spaced genomewide bins, and transposable elements.

84. The method of claim 63, wherein the region of the genome comprises a plurality of non-overlapping regions of the genome.

85. The method of claim 84, wherein the plurality of non-overlapping regions of the genome have a pre-determined size.

86. The method of claim 85, wherein the pre-determined size is about 50 kilobases (kb), about 100 kb, about 200 kb, about 500 kb, about 1 megabases (Mb), about 2 Mb, about 5 Mb, or about 10 Mb.

87. The method of claim 84, wherein the plurality of non-overlapping regions of the genome comprises at least about 1,000 distinct regions.

88. The method of claim 87, wherein the plurality of non-overlapping regions of the genome comprises at least about 2,000 distinct regions.

89. The method of claim 63, wherein determining the first or second tumor fraction comprises comparing the methylation fraction profile with one or more reference methylation fraction profiles, wherein the one or more reference methylation fraction profiles are obtained from additional cfDNA molecules obtained or derived from additional bodily fluid samples of additional subjects.

90. The method of claim 89, wherein the additional subjects comprise one or more subjects with cancer.

91. The method of claim 89, wherein the additional subjects comprise one or more subjects without cancer.

92. The method of claim 89, wherein the additional subjects comprise one or more subjects having tumor progression.

93. The method of claim 89, wherein the additional subjects comprise one or more subjects not having tumor progression.

94. The method of claim 89, wherein the one or more reference methylation fraction profiles are obtained using additional bodily fluid samples of the subject obtained at one or more subsequent time points after the first timepoint.

95. The method of claim 63, further comprising detecting that the tumor status comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5.

96. The method of claim 63, further comprising detecting a major molecular response (MMR) of the subject when the first tumor fraction or the second tumor fraction is less than 0.01, less than 0.05, less than 0.1, less than 0.2, less than 0.3, less than 0.4, or less than 0.5.

97. The method of any one of claims 63-96, further comprising detecting the tumor status of the subject with a sensitivity of at least about 50%.

98. The method of claim 97, further comprising detecting the tumor status of the subject with a sensitivity of at least about 70%.

99. The method of claim 98, further comprising detecting the tumor status of the subject with a sensitivity of at least about 90%.

100. The method of any one of claims 63-99, further comprising detecting the tumor status of the subject with a specificity of at least about 50%.

101. The method of claim 100, further comprising detecting the tumor status of the subject with a specificity of at least about 70%.

102. The method of claim 101, further comprising detecting the tumor status of the subject with a specificity of at least about 90%.

103. The method of claim 102, further comprising detecting the tumor status of the subject with a specificity of at least about 98%.

104. The method of any one of claims 63-103, further comprising detecting the tumor status of the subject with a positive predictive value (PPV) of at least about 50%.

105. The method of claim 104, further comprising detecting the tumor status of the subject with a positive predictive value (PPV) of at least about 70%.

106. The method of claim 105, further comprising detecting the tumor status of the subject with a positive predictive value (PPV) of at least about 90%.

107. The method of any one of claims 63-106, further comprising detecting the tumor status of the subject with a negative predictive value (NPV) of at least about 50%.

108. The method of claim 107, further comprising detecting the tumor status of the subject with a negative predictive value (NPV) of at least about 70%.

109. The method of claim 108, further comprising detecting the tumor status of the subject with a negative predictive value (NPV) of at least about 90%.

110. The method of any one of claims 63-109, further comprising detecting the status progression of the subject with an area under the curve (AUC) of at least about 0.60.

111. The method of claim 110, further comprising detecting the tumor status of the subject with an area under the curve (AUC) of at least about 0.75.

112. The method of claim 111, further comprising detecting the tumor status of the subject with an area under the curve (AUC) of at least about 0.90.

113. The method of any one of claims 63-112, further comprising determining a tumor non-progression of the subject when tumor progression is not detected.

114. The method of any one of claims 63-113, further comprising, based on the determined tumor status of the subject, administering a therapeutically effective dose of a second therapeutic to treat the cancer of the subject.

115. The method of claim 114, wherein the second therapeutic comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor.

116. The method of any one of claims 63-115, wherein the first and the second pluralities of cfDNA molecules are from immune cells of the subject.

117. The method of any one of claims 63-116, wherein the detected tumor status is indicative of tumor progression, non-progression, regression, or recurrence.

118. The method of any one of claims 63-117, wherein the first and second MS data are obtained by a sequencing device or computer processor.

119. The method of any one of claims 1-60 and 63-118, wherein the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.

120. A computer system for assessing tumor status of a subject with cancer, comprising:
a database that is configured to store (i) first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject, and (ii) second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; and
one or more computer processors operatively coupled to the database, wherein the one or more computer processors are individually or collectively programmed to:
determine, based on the first MS data, an average methylation fraction for each of one or more CpG islands in the region of the genome, thereby obtaining a first average methylation fraction profile;
determine, based on the second MS data, an average methylation fraction for each of one or more CpG islands in the region of the genome, thereby obtaining a second average methylation fraction profile;
compare the first average methylation fraction profile across the one or more CpG islands and the second average methylation fraction profile across the one or more CpG islands to determine a methylation fraction profile;
determine a first tumor fraction of the subject at the first timepoint or a second tumor fraction of the subject at the second timepoint, based at least in part on the respective methylation fraction profiles; and
detect a tumor status of the subject based at least in part on the first tumor fraction or the second tumor fraction.

121. A non-transitory computer-readable medium comprising machine-executable instructions which, upon execution by one or more computer processors, perform a method for assessing tumor status of a subject with cancer, the method comprising:
obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;
determining, based on the first MS data, an average methylation fraction for each of one or more CpG islands in the region of the genome, thereby obtaining a first average methylation fraction profile;
obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;
determining, based on the second MS data, an average methylation fraction for each of one or more CpG islands in the region of the genome, thereby obtaining a second average methylation fraction profile;
comparing the first average methylation fraction profile across the one or more CpG islands and the second average methylation fraction profile across the one or more CpG islands to determine a methylation fraction profile;
determining a first tumor fraction of the subject at the first timepoint or a second tumor fraction of the subject at the second timepoint, based at least in part on the respective methylation fraction profiles; and
detecting a tumor status of the subject based at least in part on the first tumor fraction or the second tumor fraction.

122. The computer system of claim 120 or the non-transitory computer-readable medium of claim 121, wherein the detected tumor progression is based at least in part on one or more statistical modeling analyses of the respective methylation fraction profiles.

123. The system or medium of claim 122, wherein the one or more statistical modeling analyses comprise linear regression, simple regression, binary regression, Bayesian linear regression, Bayesian modeling, polynomial regression, Gaussian process regression, Gaussian modeling, binary regression, logistic regression, or nonlinear regression.

124. The system or medium of claim 122 or claim 123, wherein the one or more statistical modeling analyses compare the detected tumor progression with MS data derived from a sample having a known tumor fraction, MS data derived from a pure tumor sample, or MS data derived from a healthy sample.

125. A method for assessing tumor status of a subject with cancer, comprising:
obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;
determining, based on the first MS data, a methylation profile for each of one or more loci of the genome, thereby obtaining a first methylation profile;
obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;
determining, based on the second MS data, a methylation profile for each of one or more loci of the genome, thereby obtaining a second methylation profile;
comparing the first methylation profile across the one or more loci and the second methylation profile across the one or more loci;
determining a first tumor fraction of the subject at the first timepoint or a second tumor fraction of the subject at the second timepoint, based at least in part on the respective methylation profiles; and
detecting a tumor status of the subject based at least in part on the first tumor fraction or the

126. The method of claim 125, wherein the first and the second methylation profiles comprise 5-hydroxymethylcytosine status, 5-methylcytosine status, enrichment-based methylation assessment, median methylation level, mode methylation level, maximum methylation level, or minimum methylation level.

127. The method of claim 125 or claim 126, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.

128. The method of claim 125, wherein obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads.

129. The method of claim 128, wherein the methylation sequencing comprises whole genome bisulfite sequencing.

130. The method of claim 128, wherein the methylation sequencing comprises whole genome enzymatic methyl-seq.

131. The method of claim 128, wherein the methylation sequencing comprises oxidative bisulfite sequencing, TET-assisted pyridine borane sequencing (TAPS), TET-assisted bisulfite sequencing (TABS), oxidative bisulfite sequencing (oxBS-Seq), APOBEC-coupled epigenetic sequencing (ACE-seq), methylated DNA immunoprecipitation (MeDIP) sequencing, hydroxymethylated DNA immunoprecipitation (hMeDIP) sequencing, methylation array analysis, reduced representation bisulfite sequencing (RRBS-Seq), or cytosine 5-hydroxymethylation sequencing.

132. The method of claim 128, further comprising aligning the first or second plurality of sequencing reads to a reference genome, thereby producing a plurality of aligned sequencing reads.

133. The method of claim 128, further comprising enriching the first or second plurality of cfDNA molecules for the region of the genome.

134. The method of claim 128, wherein the region of the genome comprises one or more of: CpG islands, CpG shores, patient-specific partially methylated domains, common partially methylated domains, promoters, gene bodies, evenly spaced genomewide bins, and transposable elements.

135. The method of claim 128, wherein the region of the genome comprises a plurality of non-overlapping regions of the genome.

136. The method of claim 128, wherein determining the first or second tumor fraction comprises comparing the methylation fraction profile with one or more reference methylation fraction profiles, wherein the one or more reference methylation fraction profiles are obtained from additional cfDNA molecules obtained or derived from additional bodily fluid samples of additional subjects.

137. The method of claim 128, further comprising detecting that the tumor status comprises tumor progression of the subject when the first tumor fraction or the second tumor fraction is greater than 1, greater than 1.1, greater than 1.2, greater than 1.3, greater than 1.4, greater than 1.5, greater than 1.6, greater than 1.7, greater than 1.8, greater than 1.9, greater than 2, greater than 3, greater than 4, or greater than 5.

138. The method of claim 128, further comprising detecting a major molecular response (MMR) of the subject when the first tumor fraction or the second tumor fraction is less than 0.01, less than 0.05, less than 0.1, less than 0.2, less than 0.3, less than 0.4, or less than 0.5.

139. The method of any one of claims 128-138, further comprising determining a tumor non-progression of the subject when tumor progression is not detected.

140. The method of any one of claims 128-139, further comprising, based on the determined tumor status of the subject, administering a therapeutically effective dose of a second therapeutic to treat the cancer of the subject.

141. The method of any one of claims 128-140, wherein the first and the second pluralities of cfDNA molecules are from immune cells of the subject.

142. The method of any one of claims 128-141, wherein the detected tumor status is indicative of tumor progression, non-progression, regression, or recurrence.

143. The method of any one of claims 128-142, wherein the first and second MS data are obtained by a sequencing device or computer processor.

144. The method of any one of claims 128-143, wherein the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.

145. A computer system for assessing tumor status of a subject with cancer, comprising:
a database that is configured to store (i) first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject, and (ii) second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject; and
one or more computer processors operatively coupled to the database, wherein the one or more computer processors are individually or collectively programmed to:
determine, based on the first MS data, a methylation profile for each of one or more CpG islands in the region of the genome, thereby obtaining a first methylation profile;
determine, based on the second MS data, a methylation profile for each of one or more CpG islands in the region of the genome, thereby obtaining a second methylation profile;
compare the first methylation profile across the one or more CpG islands and the second methylation profile across the one or more CpG islands;
determine a first tumor fraction of the subject at the first timepoint or a second tumor fraction of the subject at the second timepoint, based at least in part on the respective methylation profiles; and
detect a tumor status of the subject based at least in part on the first tumor fraction or the second tumor fraction.

146. A non-transitory computer-readable medium comprising machine-executable instructions which, upon execution by one or more computer processors, perform a method for assessing tumor status of a subject with cancer, the method comprising:
obtaining first methylation sequencing (MS) data of a first plurality of cell-free DNA (cfDNA) molecules across a region of a genome, wherein the first plurality of cfDNA molecules is obtained or derived from a first bodily fluid sample of the subject at a first timepoint, wherein the first timepoint precedes an administration of a therapeutic configured to treat the cancer to the subject;
determining, based on the first MS data, a methylation profile for each of one or more CpG islands in the region of the genome, thereby obtaining a first methylation profile;
obtaining second MS data of a second plurality of cell-free DNA (cfDNA) molecules across the region of the genome, wherein the second plurality of cfDNA molecules is obtained or derived from a second bodily fluid sample of the subject at a second timepoint, wherein the second timepoint is subsequent to the administration of the therapeutic to the subject;
determining, based on the second MS data, a methylation profile for each of one or more CpG islands in the region of the genome, thereby obtaining a second methylation profile;
comparing the first average methylation fraction profile across the one or more CpG islands and the second average methylation fraction profile across the one or more CpG islands;
determining a first tumor fraction of the subject at the first timepoint or a second tumor fraction of the subject at the second timepoint, based at least in part on the respective methylation profiles; and
detecting a tumor status of the subject based at least in part on the first tumor fraction or the second tumor fraction.

147. A method for assessing tumor status of a subject with cancer, comprising:
obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;
determining, based on the first WGS data, (i) a first plurality of copy number aberrations (CNAs) in the first plurality of cfDNA molecules and (ii) a first plurality of fragment lengths of the first plurality of cfDNA molecules;
obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint;
determining, based on the first MS data, an average methylation fraction for each of one or more CpG islands in the region of the genome, thereby obtaining a first average methylation fraction profile;
obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;
determining, based on the second WGS data, (iii) a second plurality of copy number aberrations (CNAs) in the second plurality of cfDNA molecules and (iv) a second plurality of fragment lengths of the second plurality of cfDNA molecules;
obtaining second MS data of a second plurality of cell-free DNA (cfDNA) molecules across the region of the genome, wherein the second plurality of cfDNA molecules is obtained or derived from a bodily fluid sample of the subject at the second timepoint;
determining, based on the second MS data, an average methylation fraction for each of one or more CpG islands in the region of the genome, thereby obtaining a second average methylation fraction profile;
comparing the first plurality of CNAs with the second plurality of CNAs to determine a CNA profile change;
determining a fragment length profile change based on the first plurality of fragment lengths and the second plurality of fragment lengths;
comparing the first average methylation fraction profile across the one or more CpG islands and the second average methylation fraction profile across the one or more CpG islands to determine a methylation fraction profile;
determining a first tumor fraction of the subject at the first timepoint or a second tumor fraction of the subject at the second timepoint, based at least in part on the CNA profile change, the fragment length profile change, and the respective methylation fraction profiles; and
detecting a tumor status of the subject based at least in part on the first tumor fraction or the second tumor fraction.

148. A method for assessing tumor status of a subject with cancer, comprising:
obtaining first whole genome sequencing (WGS) data of a first plurality of cell-free DNA (cfDNA) molecules, wherein the first plurality of cfDNA molecules is obtained or derived from a first bodily fluid sample of the subject at a first timepoint, wherein the first timepoint precedes a therapeutic configured to treat the cancer is administered to the subject;
determining, based on the first WGS data, (i) a first plurality of copy number aberrations (CNAs) in the first plurality of cfDNA molecules and (ii) a first plurality of fragment lengths of the first plurality of cfDNA molecules;
obtaining first methylation sequencing (MS) data of a first plurality of cell-free DNA (cfDNA) molecules across a region of a genome, wherein the first plurality of cfDNA molecules is obtained or derived from a bodily fluid sample of the subject at the first timepoint;
determining, based on the first MS data, a methylation profile for each of one or more loci of the genome, thereby obtaining a first methylation profile;
obtaining second whole genome sequencing (WGS) data of a second plurality of cell-free DNA (cfDNA) molecules, wherein the second plurality of cfDNA molecules is obtained or derived from a second bodily fluid sample of the subject at a second timepoint, wherein the second timepoint is subsequent to administration of the therapeutic to the subject;
determining, based on the second WGS data, (iii) a second plurality of copy number aberrations (CNAs) in the second plurality of cfDNA molecules and (iv) a second plurality of fragment lengths of the second plurality of cfDNA molecules;
obtaining second MS data of a second plurality of cell-free DNA (cfDNA) molecules across the region of the genome, wherein the second plurality of cfDNA molecules is obtained or derived from a bodily fluid sample of the subject at the second timepoint;
determining, based on the second MS data, a methylation profile for each of one or more loci of the genome, thereby obtaining a second methylation profile;
comparing the first plurality of CNAs with the second plurality of CNAs to determine a CNA profile change;
determining a fragment length profile change based on the first plurality of fragment lengths and the second plurality of fragment lengths;
comparing the first methylation profile across the one or more loci and the second methylation profile across the one or more loci;
determining a first tumor fraction of the subject at the first timepoint or a second tumor fraction of the subject at the second timepoint, based at least in part on the CNA profile change, the fragment length profile change, and the respective methylation fraction profiles; and
detecting a tumor status of the subject based at least in part on the first tumor fraction or the second tumor fraction.

149. The method of claim 148, wherein the first and the second methylation profiles comprise 5-hydroxymethylcytosine status, 5-methylcytosine status, enrichment-based methylation assessment, median methylation level, mode methylation level, maximum methylation level, or minimum methylation level.

150. The method of any one of claims 147-149, wherein the first WGS data and the first MS data are obtained from the same sample.

151. The method of any one of claims 147-149, wherein the first WGS data and the first MS data are obtained from different samples.

152. The method of any one of claims 147-151, wherein the second WGS data and the second MS data are obtained from the same sample.

153. The method of any one of claims 147-151, wherein the second WGS data and the second MS data are obtained from different samples.

154. The method of any one of claims 147-153, wherein the first or second bodily fluid sample is selected from the group consisting of: blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, and lymph fluid.

155. The method of any one of claims 147-154, wherein obtaining the first WGS data comprises sequencing the first plurality of cfDNA molecules to generate a first plurality of sequencing reads, or wherein obtaining the second WGS data comprises sequencing the second plurality of cfDNA molecules to generate a second plurality of sequencing reads.

156. The method of any one of claims 147-155, further comprising enriching the first or second plurality of cfDNA molecules for a plurality of genomic regions.

157. The method of claim 155 or claim 156, wherein determining the first plurality of CNAs comprises determining quantitative measures of CNAs at each of at each of a plurality of genomic regions of the first plurality of sequencing reads, and wherein determining the second plurality of CNAs comprises determining quantitative measures of CNAs at each of at each of the plurality of genomic regions of the second plurality of sequencing reads.

158. The method of any one of claims 147-157, wherein determining the CNA profile change comprises comparing the first plurality of CNAs and the second plurality of CNAs with a plurality of reference CNA values, wherein the plurality of reference CNA values is obtained from additional cfDNA molecules obtained or derived from additional bodily fluid samples of additional subjects.

159. The method of any one of claims 147-158, wherein the first and second WGS data are obtained by pyrosequencing, sequencing-by-synthesis, single-molecule sequencing, Nanopore sequencing, semiconductor sequencing, sequencing-by-ligation, sequencing-by-hybridization, massively parallel sequencing, chain termination sequencing, single molecule real-time sequencing, Polony sequencing, combinatorial probe anchor synthesis, or hybrid capture-based sequencing.

160. The method of any one of claims 147-159, wherein obtaining the first MS data comprises performing methylation sequencing of the first plurality of cfDNA molecules to generate a first plurality of sequencing reads, or wherein obtaining the second WGS data comprises performing methylation sequencing of the second plurality of cfDNA molecules to generate a second plurality of sequencing reads.

161. The method of any one of claims 147-160, further comprising enriching the first or second plurality of cfDNA molecules for the region of the genome.

162. The method of any one of claims 147-161, wherein the region of the genome comprises one or more of: CpG islands, CpG shores, patient-specific partially methylated domains, common partially methylated domains, promoters, gene bodies, evenly spaced genomewide bins, and transposable elements.

163. The method of any one of claims 147-162, wherein determining the first or second tumor fraction comprises comparing the methylation fraction profile with one or more reference methylation fraction profiles, wherein the one or more reference methylation fraction profiles are obtained from additional cfDNA molecules obtained or derived from additional bodily fluid samples of additional subjects.

164. The method of any one of claims 147-163, further comprising, based on the determined tumor status of the subject, administering a therapeutically effective dose of a treatment to treat the cancer of the subject.

165. The method of claim 164, wherein the treatment comprises surgery, chemotherapy, radiation therapy, targeted therapy, immunotherapy, cell therapy, an anti-hormonal agent, an antimetabolite chemotherapeutic agent, a kinase inhibitor, a methyltransferase inhibitor, a peptide, a gene therapy, a vaccine, a platinum-based chemotherapeutic agent, an antibody, or a checkpoint inhibitor.

166. The method of any one of claims 147-165, wherein the first and the second pluralities of cfDNA molecules are from immune cells of the subject.

167. The method of any one of claims 147-166, wherein the detected tumor status is indicative of tumor progression, non-progression, regression, or recurrence.

168. The method of any one of claims 147-167, wherein the first and second MS data are obtained by a sequencing device or computer processor.

169. The method of any one of claims 147-168, wherein the subject has brain cancer, bladder cancer, breast cancer, cervical cancer, colorectal cancer, endometrial cancer, esophageal cancer, gastric cancer, kidney cancer, hepatobiliary tract cancer, leukemia, liver cancer, lung cancer, lymphoma, ovarian cancer, pancreatic cancer, prostate cancer, skin cancer, stomach cancer, thyroid cancer, or urinary tract cancer.


SEQUENCE LISTING (20 sequences)

SEQ ID NO: 1 - protein, 439 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 2 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 10 aa, Artificial Sequence
  Synthetic Construct
GFTFSDSWIH

SEQ ID NO: 6 - protein, 18 aa, Artificial Sequence
  Synthetic Construct
AWISPYGGSTYYADSVKG

SEQ ID NO: 7 - protein, 9 aa, Artificial Sequence
  Synthetic Construct
RHWPGGFDY

SEQ ID NO: 8 - protein, 11 aa, Artificial Sequence
  Synthetic Construct
RASQDVSTAVA

SEQ ID NO: 9 - protein, 7 aa, Artificial Sequence
  Synthetic Construct
SASFLYS

SEQ ID NO: 10 - protein, 9 aa, Artificial Sequence
  Synthetic Construct
QQYLYHPAT

SEQ ID NO: 11 - protein, 118 aa, Artificial Sequence [sequence omitted]

SEQ ID NO: 12 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 13 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 450 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 215 aa, Artificial Sequence
  Synthetic Construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 118 aa, Artificial Sequence
  Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 20 - protein, 108 aa, Artificial Sequence
  Synthetic Construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK